SEQUENCE LISTING (123 sequences)

SEQ ID NO: 1 - protein, 152 aa, unidentified
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 160 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHVDHHHHHH

SEQ ID NO: 3 - protein, 152 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 76: note = Homoserine
  SITE 114: note = Homoserine
  SITE 147: note = Norleucine
DCDIEGKDGKQYESVLXVSIDQLLDSXKEIGSNCLXNEFNFFKRHICDANKEGXFLFRAARKLRQFLKXNSTGDFXLHLLKVSEGTTILLNCTGQVKGRKPAALGEAQPTKSLXENKSLKEQKKLNDLCFLKRLLQEIKTCWNKILXGTKEH

SEQ ID NO: 4 - protein, 152 aa, synthetic construct
  SITE 36: note = Homoserine
  SITE 76: note = Homoserine
  SITE 114: note = Homoserine
DCDIEGKDGKQYESVLMVSIDQLLDSMKEIGSNCLXNEFNFFKRHICDANKEGMFLFRAARKLRQFLKMNSTGDFXLHLLKVSEGTTILLNCTGQVKGRKPAALGEAQPTKSLXENKSLKEQKKLNDLCFLKRLLQEIKTCWNKILMGTKEH

SEQ ID NO: 5 - protein, 35 aa, synthetic construct
  SITE 17: note = Norleucine
  SITE 27: note = Norleucine
  SITE 35: note = alpha-keto-leucine
DCDIEGKDGKQYESVLXVSIDQLLDSXKEIGSNCX

SEQ ID NO: 6 - protein, 35 aa, synthetic construct
  SITE 35: note = alpha-keto-leucine
DCDIEGKDGKQYESVLMVSIDQLLDSMKEIGSNCX

SEQ ID NO: 7 - protein, 40 aa, synthetic construct
  SITE 1: note = 5-oxaproline
  SITE 19: note = Norleucine
  SITE 34: note = Norleucine
  SITE 40: note = alpha-keto-phenylalanine
XNEFNFFKRHICDANKEGXFLFRAARKLRQFLKXNSTGDX

SEQ ID NO: 8 - protein, 40 aa, synthetic construct
  SITE 1: note = 5-oxaproline
  SITE 40: note = alpha-keto-phenylalanine
XNEFNFFKRHICDANKEGMFLFRAARKLRQFLKMNSTGDX

SEQ ID NO: 9 - protein, 38 aa, synthetic construct
  SITE 1: note = 5-oxaproline
  SITE 38: note = alpha-keto-leucine
XLHLLKVSEGTTILLNCTGQVKGRKPAALGEAQPTKSX

SEQ ID NO: 10 - protein, 39 aa, synthetic construct
  SITE 1: note = 5-oxaproline
  SITE 34: note = Norleucine
XENKSLKEQKKLNDLCFLKRLLQEIKTCWNKILXGTKEH

SEQ ID NO: 11 - protein, 39 aa, synthetic construct
  SITE 1: note = 5-oxaproline
XENKSLKEQKKLNDLCFLKRLLQEIKTCWNKILMGTKEH

SEQ ID NO: 12 - protein, 75 aa, synthetic construct
  SITE 17: note = Norleucine
  SITE 27: note = Norleucine
  SITE 36: note = Homoserine
  SITE 54: note = Norleucine
  SITE 69: note = Norleucine
  SITE 75: note = alpha-keto-phenylalanine
DCDIEGKDGKQYESVLXVSIDQLLDSXKEIGSNCLXNEFNFFKRHICDANKEGXFLFRAARKLRQFLKXNSTGDX

SEQ ID NO: 13 - protein, 77 aa, synthetic construct
  SITE 1: note = 5-oxaproline
  SITE 39: note = Homoserine
  SITE 72: note = Norleucine
XLHLLKVSEGTTILLNCTGQVKGRKPAALGEAQPTKSLXENKSLKEQKKLNDLCFLKRLLQEIKTCWNKILXGTKEH

SEQ ID NO: 14-23 - skipped (10 entries)

SEQ ID NO: 24 - protein, 20 aa, synthetic construct
  VARIANT 1..20: note = This sequence may encompass 1-10 GS repeating units
GSGSGSGSGSGSGSGSGSGS

SEQ ID NO: 25 - protein, 30 aa, synthetic construct
  VARIANT 1..30: note = This sequence may encompass 1-10 GGS repeating units
GGSGGSGGSGGSGGSGGSGGSGGSGGSGGS

SEQ ID NO: 26 - protein, 40 aa, synthetic construct
  VARIANT 1..40: note = This sequence may encompass 1-10 GGGS repeating units
GGGSGGGSGGGSGGGSGGGSGGGSGGGSGGGSGGGSGGGS

SEQ ID NO: 27 - protein, 40 aa, synthetic construct
  VARIANT 1..40: note = This sequence may encompass 1-10 GGSG repeating units
GGSGGGSGGGSGGGSGGGSGGGSGGGSGGGSGGGSGGGSG

SEQ ID NO: 28 - protein, 50 aa, synthetic construct
  VARIANT 1..50: note = This sequence may encompass 1-10 GGSGG repeating units
GGSGGGGSGGGGSGGGGSGGGGSGGGGSGGGGSGGGGSGGGGSGGGGSGG

SEQ ID NO: 29 - protein, 50 aa, synthetic construct
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 30 - protein, 15 aa, synthetic construct
GGGGSGGGGSGGGGS

SEQ ID NO: 31 - protein, 20 aa, synthetic construct
GGGGSGGGGSGGGGSGGGGS

SEQ ID NO: 32 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 33 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 447 aa, synthetic construct
QVQLVQSGAEVKKPGSSVKVSCKASGGTFSSYAISWVRQAPGQGLEWMGLIIPMFDTAGYAQKFQGRVAITVDESTSTAYMELSSLRSEDTAVYYCARAEHSSTGTFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 214 aa, synthetic construct
DIQMTQSPSSVSASVGDRVTITCRASQGISSWLAWYQQKPGKAPKLLISAASSLQSGVPSRFSGSGSGTDFTLTISSLQPEDFATYYCQQANHLPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 452 aa, synthetic construct
QGQLVQSGAEVKKPGASVKVSCKASGYTFTDYEMHWVRQAPIHGLEWIGVIESETGGTAYNQKFKGRVTITADKSTSTAYMELSSLRSEDTAVYYCAREGITTVATTYYWYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 219 aa, synthetic construct
DVVMTQSPLSLPVTLGQPASISCRSSQSIVHSNGNTYLEWYLQKPGQSPQLLIYKVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 443 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 214 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 444 aa, synthetic construct
EVQLLESGGVLVQPGGSLRLSCAASGFTFSNFGMTWVRQAPGKGLEWVSGISGGGRDTYFADSVKGRFTISRDNSKNTLYLQMNSLKGEDTAVYYCVKWGNIYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 214 aa, synthetic construct
DIQMTQSPSSLSASVGDSITITCRASLSINTFLNWYQQKPGKAPNLLIYAASSLHGGVPSRFSGSGSGTDFTLTIRTLQPEDFATYYCQQSSNTPFTFGPGTVVDFRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42-45 - skipped (4 entries)

SEQ ID NO: 46 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 119 aa, synthetic construct
QVQLVQSGVEVKKPGASVKVSCKASGYTFTNYYMYWVRQAPGQGLEWMGGFPSNGGTNFNEKFKNRVTLTTDSSTTTAYMELKSLQFDDTAVYYCARRDYRFDMGFDYWGQGTTVTVSS

SEQ ID NO: 49 - protein, 111 aa, synthetic construct [sequence omitted]

SEQ ID NO: 50 - protein, 113 aa, synthetic construct [sequence omitted]

SEQ ID NO: 51 - protein, 107 aa, synthetic construct [sequence omitted]

SEQ ID NO: 52 - protein, 459 aa, synthetic construct
QVQLVQSGGGLVQPGGSLRLSCAASGFTFSSYWMYWVRQVPGKGLEWVSAIDTGGGRTYYADSVKGRFAISRVNAKNTMYLQMNSLRAEDTAVYYCARDEGGGTGWGVLKDWPYGLDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 214 aa, synthetic construct
QPVLTQPLSVSVALGQTARITCGGNNIGSKNVHWYQQKPGQAPVLVIYRDSNRPSGIPERFSGSNSGNTATLTISRAQAGDEADYYCQVWDSSTAVFGTGTKLTVLQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 439 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCASNGDHWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 55 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 443 aa, synthetic construct
EVQLLESGGGLVQPGGSLRLSCAASGFTFSSYDMSWVRQAPGKGLEWVSTISGGGSYTYYQDSVKGRFTISRDNSKNTLYLQMNSLRAEDTAVYYCASPYYAMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 214 aa, synthetic construct
DIQLTQSPSFLSAYVGDRVTITCKASQDVGTAVAWYQQKPGKAPKLLIYWASTLHTGVPSRFSGSGSGTEFTLTISSLQPEDFATYYCQHYSSYPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 442 aa, synthetic construct
OVQLVESGGGLVKPGGSLRLSCAASGFTFSNYGMSWIROAPGKGLEWVSTISGGGSNIYYADSVKGRFTISRDNAKNSLYLQMNSLRAEDTAVYYCVSYYYGIDFWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCOOHYTIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 445 aa, synthetic construct
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 61 - protein, 218 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 444 aa, synthetic construct
QVQLVQSGAEVKKPGASVKVSCKASGYTFTSYWINWVRQAPGQGLEWMGNIYPGSSLTNYNEKFKNRVTMTRDTSTSTVYMELSSLRSEDTAVYYCARLSTGTFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 220 aa, synthetic construct
DIVMTQSPDSLAVSLGERATINCKSSQSLWDSGNQKNFLTWYQQKPGQPPKLLIYWTSYRESGVPDRFSGSGSGTDFTLTISSLQAEDVAVYYCQNDYFYPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 443 aa, synthetic construct
EVQLVQSGAEVKKPGESLRISCKGSGYTFTTYWMHWVRQATGQGLEWMGNIYPGTGGSNFDEKFKNRVTITADKSTSTAYMELSSLRSEDTAVYYCTRWTTGTGAYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 220 aa, synthetic construct
EIVLTQSPATLSLSPGERATLSCKSSQSLLDSGNQKNFLTWYQQKPGQAPRLLIYWASTRESGVPSRFSGSGSGTDFTFTISSLEAEDAATYYCQNDYSYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 450 aa, synthetic construct
QVQLVQSGAEVKKPGSSVKVSCKASGGTFSSYAISWVRQAPGQGLEWMGGIIPIFDTANYAQKFQGRVTITADESTSTAYMELSSLRSEDTAVYYCARPGLAAAYDTGSLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - protein, 214 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRNYWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 496 aa, synthetic construct
DIQMTQSPSSLSASVGDRVTITCRASQDVSSVVAWYQQKPGKAPKLLIYSASYRYTGVPSRFSGSGSGTDFTLTISSLQPEDFATYYCQQHYSTPWTFGGGTKLEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 271 aa, synthetic construct
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVQSGAEVKKPGASVKVSCKASGYTFTDYNMDWVRQAPGQGLEWMGDINPDNGVTIYNQKFEGRVTMTTDTSTSTAYMELRSLRSDDTAVYYCAREADYFYFDYWGQGTTLTVSSGGCGGGKVAACKEKVAALKEKVAALKEKVAALKE

SEQ ID NO: 70 - protein, 447 aa, synthetic construct
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 213 aa, synthetic construct
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 113 aa, synthetic construct
QVQLVESGGGVVQPGRSLRLTCKASGLTFSSSGMHWVRQAPGKGLEWVAVIWYDGSKRYYADSVKGRFTISRDNSKNTLFLQMNSLRAEDTAVYYCATNNDYWGQGTLVTVSS

SEQ ID NO: 73 - protein, 107 aa, synthetic construct
EIVLTQSPATLSLSPGERATLSCRASQSVSSYLAWYQQKPGQAPRLLIYTASNRATGIPARFSGSGSGTDFTLTISSLEPEDFAVYYCQQYSNWPRTFGQGTKVEIK

SEQ ID NO: 74 - protein, 120 aa, synthetic construct
EVQLQQSGPVLVKPGASVKMSCKASGYTFTSYYMYWVKQSHGKSLEWIGGVNPSNGGTNFNEKFKSKATLTVDKSSSTAYMELNSLTSEDSAVYYCARRDYRYDMGFDYWGQGTTLTVSS

SEQ ID NO: 75 - protein, 111 aa, synthetic construct
QIVLTQSPAIMSASPGEKVTMTCRASKGVSTSGYSYLHWYQQKPGSSPRLLIYLASYLESGVPVRFSGSGSGTSYSLTISRMEAEDAATYYCQHSRELPLTFGTGTRLEIK

SEQ ID NO: 76 - protein, 120 aa, synthetic construct
QVQLVQSGAEVKKPGASVKVSCKASGYTFTSYYMYWVRQAPGQGLEWMGGVNPSNGGTNFNEKFKSRVTITADKSTSTAYMELSSLRSEDTAVYYCARRDYRYDMGFDYWGQGTTVTVSS

SEQ ID NO: 77 - protein, 111 aa, synthetic construct
EIVLTQSPATLSLSPGERATISCRASKGVSTSGYSYLHWYQQKPGQAPRLLIYLASYLESGVPARFSGSGSGTDFTLTISSLEPEDFATYYCQHSRELPLTFGTGTKVEIK

SEQ ID NO: 78 - protein, 448 aa, synthetic construct
EIQLVQSGAEVKKPGSSVKVSCKASGYTFTHYGMNWVRQAPGQGLEWVGWVNTYTGEPTYADDFKGRLTFTLDTSTSTAYMELSSLRSEDTAVYYCTREGEGLGFGDWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 219 aa, synthetic construct
DVVMTQSPLSLPVTPGEPASISCRSSQSIVHSHGDTYLEWYLQKPGQSPQLLIYKVSNRFSGVPDRFSGSGSGTDFTLKISRVEAEDVGVYYCFQGSHIPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 5 aa, synthetic construct
NYYMY

SEQ ID NO: 81 - protein, 17 aa, synthetic construct
GINPSNGGTNFNEKFKN

SEQ ID NO: 82 - protein, 11 aa, synthetic construct
RDYRFDMGFDY

SEQ ID NO: 83 - protein, 15 aa, synthetic construct
RASKGVSTSGYSYLH

SEQ ID NO: 84 - protein, 7 aa, synthetic construct
LASYLES

SEQ ID NO: 85 - protein, 9 aa, synthetic construct
QHSRDLPLT

SEQ ID NO: 86 - protein, 5 aa, synthetic construct
NSGMH

SEQ ID NO: 87 - protein, 17 aa, synthetic construct
VIWYDGSKRYYADSVKG

SEQ ID NO: 88 - protein, 4 aa, synthetic construct
NDDY

SEQ ID NO: 89 - protein, 11 aa, synthetic construct
RASQSVSSYLA

SEQ ID NO: 90 - protein, 7 aa, synthetic construct
DASNRAT

SEQ ID NO: 91 - protein, 9 aa, synthetic construct
QQSSNWPRT

SEQ ID NO: 92 - protein, 9 aa, synthetic construct
FTFSNYGMS

SEQ ID NO: 93 - protein, 10 aa, synthetic construct
TISGGGSNIY

SEQ ID NO: 94 - protein, 9 aa, synthetic construct
VSYYYGIDF

SEQ ID NO: 95 - protein, 11 aa, synthetic construct
KASQDVTTAVA

SEQ ID NO: 96 - protein, 7 aa, synthetic construct
WASTRHT

SEQ ID NO: 97 - protein, 9 aa, synthetic construct
QQHYTIPWT

SEQ ID NO: 98 - protein, 8 aa, synthetic construct
GLTFSSSG

SEQ ID NO: 99 - protein, 8 aa, synthetic construct
IWYDGSKR

SEQ ID NO: 100 - protein, 6 aa, synthetic construct
ATNNDY

SEQ ID NO: 101 - protein, 11 aa, synthetic construct
RASQSVSSYLA

SEQ ID NO: 102 - protein, 7 aa, synthetic construct
TASNRAT

SEQ ID NO: 103 - protein, 9 aa, synthetic construct
QQYSNWPRT

SEQ ID NO: 104 - protein, 831 aa, synthetic construct
MQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQSKYGPPCPSCPAPEFLGGPSVFLFPPKPKDTLMISRTPEVTCWVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGKIEGRMDQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVLMQIPQAPWPWWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 105 - protein, 230 aa, synthetic construct
  VARIANT 18: note = Any naturally occurring amino acid
  VARIANT 19: note = Any naturally occurring amino acid
  VARIANT 21: note = Any naturally occurring amino acid
  VARIANT 140: note = Any naturally occurring amino acid
  VARIANT 142: note = Any naturally occurring amino acid
  VARIANT 177: note = Any naturally occurring amino acid
PKSCDKTHTCPPCPAPEXXGXPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKXTPPVLDSDGSFFLYSKLTVDKSRWQQGNVFSCSVMHEALHNHYTQKSLSLSPG

SEQ ID NO: 106 - protein, 48 aa, synthetic construct
QETNPTENLYFQQKNMQCQRRFYEALHDPNLNEEQRNARIRSIRDDDC

SEQ ID NO: 107 - protein, 61 aa, synthetic construct
QTADNQKNMQCQRRFYEALHDPNLNEEQRNARIRSIRDDCSQSANLLAEAQQLNDAQAPQA

SEQ ID NO: 108 - protein, 38 aa, synthetic construct
QETKNMQCQRRFYEALHDPNLNEEQRNARIRSIRDDDC

SEQ ID NO: 109 - protein, 38 aa, synthetic construct
QETFNKQCQRRFYEALHDPNLNEEQRNARIRSIRDDDC

SEQ ID NO: 110 - protein, 38 aa, synthetic construct
QETFNMQCQRRFYEALHDPNLNKEQRNARIRSIRDDDC

SEQ ID NO: 111 - protein, 37 aa, synthetic construct
QETFNMQCQRRFYEALHDPNLNEEQRNARIRSIKDDC

SEQ ID NO: 112 - protein, 35 aa, synthetic construct
QETMQCQRRFYEALHDPNLNEEQRNARIRSIKDDC

SEQ ID NO: 113 - protein, 34 aa, synthetic construct
QETQCQRRFYEALHDPNLNEEQRNARIRSIKDDC

SEQ ID NO: 114 - protein, 33 aa, synthetic construct
QETCQRRFYEALHDPNLNEEQRNARIRSIKDDC

SEQ ID NO: 115 - protein, 20 aa, synthetic construct
QETRGNCAYHKGQLVWCTYH

SEQ ID NO: 116 - protein, 20 aa, synthetic construct
QETRGNCAYHKGQIIWCTYH

SEQ ID NO: 117 - protein, 133 aa, Homo sapiens [sequence omitted]

SEQ ID NO: 118 - protein, 170 aa, Homo sapiens
YFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 119 - skipped

SEQ ID NO: 120 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 121 - protein, 6 aa, synthetic construct
  VARIANT 6: note = alanine or proline
LLQGPX

SEQ ID NO: 122 - protein, 8 aa, synthetic construct
GGLLQGPP

SEQ ID NO: 123 - protein, 17 aa, synthetic construct
  SITE 1: note = Acetyl arginine
  DISULFID 4..14: note = Intrachain disulfide bond
  SITE 8: note = Lysine-3-((3-((2,5-dioxopyrrolidin-1-yl)oxy)-3-oxop ropyl)disulfaneyl)propanoate
  SITE 17: note = Histidine amide
RGNCAYHKGQLVWCTYH

(19) United States
(12) Patent Application Publication
(10) Pub. No.: US 2024/0158537 A1
(43) Pub. Date: May 16, 2024

(54) SYNTHETIC IL-7 AND IL-7 IMMUNOCYTOKINES

(71) Applicant: Bright Peak Therapeutics AG, Basel (CH)

(72) Inventors: Vijaya Raghavan PATTABIRAMAN, Volketswil (CH); Bertolt KREFT, Kleinmachnow (DE); Jean-Philippe CARRALOT, Saint-Louis (FR); Rubén ALVAREZ SANCHEZ, Rosenau (FR); Magali MULLER, Pulversheim (FR); Matilde ARÉVALO-RUIZ, Basel (CH)

(21) Appl. No.: 17/861,179

(22) Filed: Jul. 9, 2022

Related U.S. Application Data
(60) Provisional application No. 63/219,981, filed on Jul. 9, 2021.
(60) Provisional application No. 63/219,989, filed on Jul. 9, 2021.

(51) Int. Cl. C07K 16/46; C07K 14/00; C07K 14/54; C07K 16/28

(52) U.S. Cl. CPC C07K 16/46; C07K 14/001; C07K 14/5418; C07K 16/2818

(57) ABSTRACT

The present disclosure relates to modified anti-PD-1 polypeptides linked to IL-7, pharmaceutical compositions comprising modified anti-PD-1 polypeptides linked to IL-7, methods of making anti-PD-1 polypeptides linked to IL-7, and methods of using the modified anti-PD-1 polypeptides linked to IL-7 for treatment of diseases. In one aspect, the disclosure relates to methods of treating cancer in a subject using the modified anti-PD-1 polypeptides linked to IL-7. Also provided herein is synthetic IL-7 and methods of manufacture thereof.

### CROSS REFERENCE

This application claims the benefit of U.S. Provisional Application No. 63/219,981 filed Jul. 9, 2021, and U.S. Provisional Application No. 63/219,989 filed Jul. 9, 2021, which applications are incorporated herein by reference in its entirety.

### SEQUENCE LISTING

The instant application contains a Sequence Listing which has been submitted electronically in XML file format and is hereby incorporated by reference in its entirety. Said XML copy, created on Aug. 18, 2022, is named 94917-0049_728201US_SL.xml and is 132,248 bytes in size.

### BACKGROUND

In 2021, an estimated 1.8 million new cases of cancer will be diagnosed in the United States, and over 600,000 people will die from the disease. Immunotherapies utilize the immune system of a subject to aid in the treatment of ailments. Immunotherapies can be designed to either activate or suppress the immune system depending on the nature of the disease being treated. A goal of various immunotherapies for the treatment of cancer is to stimulate the immune system so that it recognizes and destroys tumors or other cancerous tissue.

Programmed cell death protein 1 (PD-1) is a protein on the surface of cells that regulates the immune system's response to cells of the human body by downregulating the immune system and promoting self-tolerance by suppressing T cell inflammatory activity. Programmed cell death-ligand 1 (PD-L1) is a type 1 transmembrane protein that suppresses the adaptive arm of the immune system. The PD-1 and PD-L1 pathways represent adaptive immune system resistance mechanisms exerted by tumor cells in response to endogenous immune anti-tumor activity. PD-1 inhibitors, such as anti-PD-1 polypeptides and anti-PD-1 antigen binding fragments are checkpoint inhibitor anticancer agents that block the activity of PD-1 immune checkpoint proteins. Single mechanism therapies alone, however, in many instances are insufficient for treating cancer. Thus, there is a need for improved tools for cancer therapy.

### BRIEF SUMMARY

Described herein are anti-programmed cell death protein 1 (PD-1)-interleukin 7 (IL-7) immunocytokines and uses thereof. Also provided herein are synthetic IL-7 polypeptides, methods of making the same, and methods of making immunocytokine compositions comprising the same.

In one aspect herein is a composition comprising: a polypeptide which selectively binds to programmed cell death protein 1 (PD-1); an IL-7 polypeptide; and a linker, wherein the linker comprises: a first point of attachment covalently attached to the IL-7 polypeptide; and a second point of attachment covalently attached to a non-terminal residue of the polypeptide which selectively binds to PD-1.

In another aspect herein is a composition comprising: a polypeptide which selectively binds to programmed cell death protein 1 (PD-1); an IL-7 polypeptide; and a linker, wherein the linker is a chemical linker, and wherein the linker comprises: a first point of attachment covalently attached to the IL-7 polypeptide; and a second point of attachment covalently attached to the polypeptide which selectively binds to PD-1.

In another aspect herein is a composition comprising: (a) an anti-PD-1 antibody or antigen binding fragment and that comprises an Fc region; (b) a linker covalently attached to the Fc region at an amino acid residue selected from the group consisting of (Eu numbering): (i) Lys 246; (ii) Lys 248; (iii) Lys 288; (iv) Lys 290; and (v) Lys 317; an (c) an IL-7 polypeptide covalently attached to the linker.

In another aspect herein is a synthetic IL-7 polypeptide, comprising a homoserine (Hse) residue at a position selected from a region of residues 31-41, a region of residues 71-81, or a region of residues 109-119, wherein residue position numbering is based on SEQ ID NO: 1 as a reference sequence.

In yet another aspect provided herein is a method of making a synthetic IL-7 polypeptide, comprising: a) synthesizing two or more fragments of the synthetic IL-7 polypeptide; b) ligating the fragments; and c) folding the ligated fragments.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawing, of which:

FIG. 1A illustrates the signaling pathway of IL-7 and the IL-7 receptor.

FIG. 1B shows differential expression of CD127 (IL-7R) on a variety of T cell subtypes.

FIG. 2 shows a general synthesis scheme used to produce synthetic IL-7 linear and folded proteins.

FIG. 3A shows characterization data for a synthetic IL-7 polypeptide tri-depsipeptide of SEQ ID NO: 3-Linear protein, including a RP-HPLC trace, showing retention time on the X-axis and absorbance on the Y-axis (top left); and an ESI-HRMS trace, with molecular weight on the X-axis and intensity on the Y-axis (top right).

FIG. 3B shows characterization data of a folded IL-7 polypeptide of SEQ ID NO: 3 linear protein, including a RP-HPLC trace, showing retention time on the X-axis and intensity on the Y-axis (top); and a deconvoluted matrix assisted laser desorption ionization-time of flight (MALDI-TOF) mass spectrometry (MS) trace, with molecular weight on the X-axis and percentage of abundance on the Y-axis (bottom).

FIG. 3C shows characterization data for a synthetic IL-7 polypeptide tri-depsipeptide of SEQ ID NO: 3-Linear protein with azide conjugation handle on N-terminus, including a RP-HPLC trace, showing retention time on the X-axis and absorbance on the Y-axis (top left); and an ESI-HRMS trace, with molecular weight on the X-axis and intensity on the Y-axis (top right).

FIG. 3D shows characterization data of a folded IL-7 polypeptide of SEQ ID NO: 3 with azide conjugation handle in N-terminus (Composition AA), including a RP-HPLC trace, showing retention time on the X-axis and intensity on the Y-axis (top left); and an ESI-HRMS trace, with molecular weight on the X-axis and intensity on the Y-axis (top right).

FIG. 4A shows a 3D representation of an IL-7 polypeptide.

FIG. 4B shows a 3D representation of an IL-7 polypeptide having an azide conjugation handle attached to the N-terminus of an IL-7 polypeptide (e.g., Composition AA).

FIG. 4C shows a 3D representation of an IL-7 polypeptide having a polymer attached to the N-terminus. The polymer can be attached through an azide conjugation handle (such as that shown in FIG. 4B) reaction with a polymer comprising an alkyne (e.g., DBCO-PEG).

FIG. 5A shows site-selective modification of anti-PD-1 antibody by AJICAP technology to introduce one conjugation handle.

FIG. 5B shows site-selective conjugation reaction of IL7 cytokine to generate anti-PD-1-IL7 with DAR1 or DAR 2.

FIG. 6A shows ELISA assay results of dose dependent binding to PD1 of SEQ ID NOs: 46-47, Composition A and B.

FIG. 6B shows ELISA assay results of dose dependent binding to PD1 of SEQ ID NOs: 76-77, Composition C.

FIG. 7 shows the dose dependent ability of SEQ ID NOs: 76-77 and Composition C to block PD1 signaling in a PD1/PDL1 blocking assay.

FIG. 8A shows ELISA results of dose dependent binding to human FcRn of SEQ ID NOs: 76-77 and Composition C.

FIG. 8B shows ELISA results of dose dependent binding to mouse FcRn of SEQ ID NOs: 76-77 and Composition C.

FIG. 9A shows dose dependent STAT5 phosphorylation in CD8 memory and naïve T cells of SEQ ID NOs: 2 and 3.

FIG. 9B shows dose dependent STAT5 phosphorylation in CD8 memory and naïve T cells of Composition A and C and SEQ ID NO: 3.

FIG. 10A shows changes in relative mouse body weight over a two week period % (y-axis) while weekly dosing of SEQ ID NOs: 76-77 (10 mg/kg) and Composition C (1, 3, and 10 mg/kg). Arrows indicate dosing days.

FIG. 10B shows relative tumor volume % (y-axis) over a two week period while weekly dosing of SEQ ID NOs: 76-77 (10 mg/kg) and Composition C (1, 3 and 10 mg/kg). Arrows indicate dosing days.

### DETAILED DESCRIPTION

In one aspect, disclosed herein are IL-7 polypeptides and immunocytokine compositions comprising IL-7 polypeptides. In some instances, IL-7 polypeptides of the immunocytokine compositions are synthetic (e.g., synthesized chemically). Such chemical synthesis, in some embodiments, allows for the IL-7 polypeptides to be site specifically incorporated into an immunocytokine composition owing to the ability to place a conjugation handle on the IL-7 at a desired location (e.g., the N-terminus of the IL-7) during the synthesis.

Also provided herein are synthetic IL-7 polypeptides. Synthetic IL-7 polypeptides can be manufactured according to the methods provided herein. In some embodiments, synthetic IL-7 polypeptide mimics the tertiary structure of a recombinant or wild type IL-7 and, in some instance, displays a substantially similar activity to wild type IL-7. In some instances, synthetic IL-7 is able to effectuate signaling via the IL-7 receptor (IL-7R) in a substantially similar way to wild type or recombinant IL-7. In some instances, the IL-7 polypeptides can be attached to an additional polypeptide, such as an antibody. The synthetic IL-7s and corresponding methods of manufacturing synthetic IL-7 and immunocytokines comprising IL-7s can be used to generate immunocytokines comprising IL-7 and any antibody, including the anti-PD-1 antibodies provided herein.

Also disclosed herein are anti-PD-1 polypeptides. In some embodiments, the anti-PD-1 polypeptides are conjugated to a cell-signaling molecule, such as IL-7. The anti-PD-1-IL-7 immunocytokines of the disclosure can have synergistic efficacy and improved tolerability by a subject. In some embodiments, the anti-PD-1-IL-7 immunocytokines may significantly reduce the therapeutic dose of the anti-PD-1 polypeptide or IL-7 for a subject with a disease, such as cancer.

The anti-PD-1-IL-7 immunocytokines can act by one or more modes of action. In some embodiments, the anti-PD-1-IL-7 immunocytokines can inhibit PD-1 by targeting PD-1 on for instance CD8+ T cells within tumors. In some embodiments, the anti-PD-1-IL-7 immunocytokines can activate T cells via IL-7R.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The section headings used herein are for organizational purposes only and are not to be construed as limiting the subject matter described.

### Definitions

All terms are intended to be understood as they would be understood by a person skilled in the art. Unless defined otherwise, all technical and scientific terms used herein have the same meaning as commonly understood by one of ordinary skill in the art to which the disclosure pertains.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

The terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Referred to herein are groups which are “attached” or “covalently attached” to residues of polypeptides. As used herein, “attached” or “covalently attached” means that the group is tethered to the indicated reside, and such tethering can include a linking group (i.e., a linker). Thus, for a group “attached” or “covalently attached” to a residue, it is expressly contemplated that such linking groups are also encompassed.

As used herein, an “alpha-keto amino acid” or the phrase “alpha-keto” before the name of an amino acid refers to an amino acid or amino acid derivative having a ketone functional group positioned between the carbon bearing the amino group and the carboxylic acid of an amino acid. Alpha-keto amino acids of the instant disclosure have a structure as set forth in the following formula:

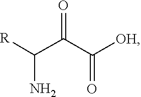

wherein R is the side chain of any natural or unnatural amino acid. The R functionality can be in either the L or D orientation in accordance with standard amino acid nomenclature. In preferred embodiments, alpha-keto amino acids are in the L orientation. When the phrase “alpha-keto” is used before the name of a traditional natural amino acid (e.g., alpha-keto leucine, alpha-keto phenylalanine, etc.) or a common unnatural amino acid (e.g., alpha-keto norleucine, alpha-keto O-methyl-homoserine, etc.), it is intended that the alpha-keto amino acid referred to matches the above formula with the side chain of the referred to amino acid. When an alpha-keto amino acid residue is set forth in a peptide or polypeptide sequence herein, it is intended that a protected version of the relevant alpha-keto amino acid is also encompassed (e.g., for a sequence terminating in a C-terminal alpha-keto amino acid, the terminal carboxylic acid group may be appropriately capped with a protecting group such as a tert-butyl group, or the ketone group with an acetal protecting group). Other protecting groups encompassed are well known in the art.

Binding affinity refers to the strength of a binding interaction between a single molecule and its ligand/binding partner. A higher binding affinity refers to a higher strength bond than a lower binding affinity. In some instances, binding affinity is measured by the dissociation constant (K_{D}) between the two relevant molecules. When comparing K_{D} values, a binding interaction with a lower value will have a higher binding affinity than a binding interaction with a higher value. For a protein-ligand interaction, K_{D} is calculated according to the following formula:

$${K}_{D} = \frac{[ L ] [ P ]}{[ L P ]}$$

where [L] is the concentration of the ligand, [P] is the concentration of the protein, and [LP] is the concentration of the ligand/protein complex.

Referred to herein are certain amino acid sequences (e.g., polypeptide sequences) which have a certain percent sequence identity to a reference sequence or refer to a residue at a position corresponding to a position of a reference sequence. Sequence identity is measured by protein-protein BLAST algorithm using parameters of Matrix BLOSUM62, Gap Costs Existence:11, Extension:1, and Compositional Adjustments Conditional Compositional Score Matrix Adjustment. This alignment algorithm is also used to assess if a residue is at a “corresponding” position through an analysis of the alignment of the two sequences being compared.

Unless otherwise specified, is contemplated that “protected” versions of amino acids (e.g., those containing a chemical protecting group affixed to a functionality of the amino acid, particularly a side chain of the amino acid but also at another point of the amino acid) qualify as the same amino acid as the “unprotected” version for sequence identity purposes, particularly for chemically synthesized polypeptides. It is also contemplated that such protected versions are also encompassed by the SEQ ID NOs provided herein. Non-limiting examples of protecting groups which may be encompassed include fluorenylmethyloxycarbonyl (Fmoc), triphenylmethyl (trityl or trt), tert-Butyloxycarbonyl (Boc), 2,2,4,6,7-pentamethyldihydrobenzofuran-5-sulfonyl (Pbf), acetamidomethyl (Acm), tert-butyl (tBu or OtBu), 2,2-dimethyl-1-(4-methoxyphenyl)propane-1,3-diol ketal or acetal, and 2,2-dimethyl-1-(2-nitrophenyl)propane-1,3-diol ketal or acetal. Other protecting groups well known in the art are also encompassed. Similarly, modified versions of natural amino acids are also intended to qualify as natural version of the amino acid for sequence identity purposes. For example, an amino acid comprising a side chain heteroatom which can be covalently modified (e.g., to add a conjugation handle, optionally through a linker), such as a lysine, glutamine, glutamic acid, asparagine, aspartic acid, cysteine, or tyrosine, which has been covalently modified would be counted as the base amino acid (see, e.g., Structure 2 below, which would be counted as a lysine for sequence identity and SEQ ID purposes). Similarly, an amino acid comprising another group added to the C- or N-terminus would be counted as the base amino acid.

In some instances, peptides provided herein may be depsipeptides. For example, a depsipeptide linkage results from certain ligation reactions described herein (e.g., KAHA ligations) during the synthesis of synthetic IL-7s and relevant precursor peptides. In particular, hydroxyl containing amino acids (e.g., serine, threonine, and homoserine) form depsipeptide linkages with the adjacent amino acid on the N-terminal side. Thus, when a sequence ID lists an amino acid sequence, it is also contemplated that a depsipeptide version of the sequence is also encompassed, particularly at homoserine residues.

The term “pharmaceutically acceptable” refers to approved or approvable by a regulatory agency of the Federal or a state government or listed in the U.S. Pharmacopeia or other generally recognized pharmacopeia for use in animals, including humans.

A “pharmaceutically acceptable excipient, carrier or diluent” refers to an excipient, carrier or diluent that can be administered to a subject, together with an agent, and which does not destroy the pharmacological activity thereof and is nontoxic when administered in doses sufficient to deliver a therapeutic amount of the agent.

A “pharmaceutically acceptable salt” suitable for the disclosure may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, sulfanilic, formic, toluenesulfonic, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC—(CH₂)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts include those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

Certain formulas and other illustrations provided herein depict triazole reaction products resulting from azide-alkyne cycloaddition reactions. While such formulas generally depict only a single regioisomer of the resulting triazole formed in the reaction, it is intended that the formulas encompass both resulting regioisomers. Thus, while the formulas depict only a single regioisomer (e.g.

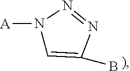

it is intended that the other regioisomer (e.g.

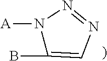

is also encompassed.

The term “subject” refers to an animal which is the object of treatment, observation, or experiment. By way of example only, a subject includes, but is not limited to, a mammal, including, but not limited to, a human or a non-human mammal, such as a non-human primate, bovine, equine, canine, ovine, or feline.

The term “optional” or “optionally” denotes that a subsequently described event or circumstance can but need not occur, and that the description includes instances where the event or circumstance occurs and instances in which it does not.

The term “moiety” refers to a specific segment or functional group of a molecule. Chemical moieties are often recognized chemical entities embedded in or appended to a molecule.

As used herein, the term “number average molecular weight” (Mn) means the statistical average molecular weight of all the individual units in a sample, and is defined by Formula (1):

$$Mn = \frac{\sum {N}_{i} {M}_{i}}{\sum {N}_{i}} \qquad Formula ( 1 )$$

where Mᵢ is the molecular weight of a unit and Nᵢ is the number of units of that molecular weight.

As used herein, the term “weight average molecular weight” (Mw) means the number defined by Formula (2):

$$Mw = \frac{\sum {N}_{i} {M}_{i}^{2}}{\sum {N}_{i} {M}_{i}} \qquad Formula ( 2 )$$

where Mᵢ is the molecular weight of a unit and Nᵢ is the number of units of that molecular weight.

As used herein, “peak molecular weight” (Mp) means the molecular weight of the highest peak in a given analytical method (e.g., mass spectrometry, size exclusion chromatography, dynamic light scattering, analytical centrifugation, etc.).

As used herein, “non-canonical” amino acids can refer to amino acid residues in D- or L-form that are not among the 20 canonical amino acids generally incorporated into naturally occurring proteins.

As used herein, “conjugation handle” refers to a reactive group capable of forming a bond upon contacting a complementary reactive group. In some instances, a conjugation handle preferably does not have a substantial reactivity with other molecules which do not comprise the intended complementary reactive group. Non-limiting examples of conjugation handles, their respective complementary conjugation handles, and corresponding reaction products can be found in the table below. While table headings place certain reactive groups under the title “conjugation handle” or “complementary conjugation handle,” it is intended that any reference to a conjugation handle can instead encompass the complementary conjugation handles listed in the table (e.g., a trans-cyclooctene can be a conjugation handle, in which case tetrazine would be the complementary conjugation handle). In some instances, amine conjugation handles and conjugation handles complementary to amines are less preferable for use in biological systems owing to the ubiquitous presence of amines in biological systems and the increased likelihood for off-target conjugation.

Table of Conjugation Handles

| | | Reaction |
|---|---|---|
| Conjugation Handle | Complementary Conjugation Handle | Product |
| Sulfhydryl | alpha-halo-carbonyl (e.g., bromoacetamide), alpha- | thioether |
| | beta unsaturated carbonyl (e.g., maleimide, | |
| | acrylamide) | |
| Azide | alkyne (e.g., terminal alkyne, substituted | triazole |
| | cyclooctyne (e.g., dibenzocycloocytne (DBCO), | |
| | difluorocyclooctyne, bicyclo[6.1.0]nonyne, etc.)) | |
| Phosphine | Azide/ester pair | amide |
| Tetrazine | trans-cyclooctene | dihydropyridazine |
| Amine | Activated ester (e.g., N-hydroxysuccinimide ester, | amide |
| | pentaflurophenyl ester) | |
| isocyanate | amine | urea |
| epoxide | amine | alkyl-amine |
| hydroxyl amine | aldehyde, ketone | oxime |
| hydrazide | aldehyde, ketone | hydrazone |
| potassium acyl | O-substituted hydroxylamine (e.g., | amide |
| trifluoroborate | O-carbamoylhydroxylamine) | |

Throughout the instant application, prefixes are used before the term “conjugation handle” to denote the functionality to which the conjugation handle is linked. For example, a “protein conjugation handle” is a conjugation handle attached to a protein (either directly or through a linker), an “antibody conjugation handle” is a conjugation handle attached to an antibody (either directly or through a linker), and a “linker conjugation handle” is a conjugation handle attached to a linker group (e.g., a bifunctional linker used to link a synthetic protein and an antibody).

The term “alkyl” refers to a straight or branched hydrocarbon chain radical, having from one to twenty carbon atoms, and which is attached to the rest of the molecule by a single bond. An alkyl comprising up to 10 carbon atoms is referred to as a C₁-C₁₀ alkyl, likewise, for example, an alkyl comprising up to 6 carbon atoms is a C₁-C₆ alkyl. Alkyls (and other moieties defined herein) comprising other numbers of carbon atoms are represented similarly. Alkyl groups include, but are not limited to, C₁-C₁₀ alkyl, C₁-C₉ alkyl, C₁-C₆ alkyl, C₁-C₇ alkyl, C₁-C₆ alkyl, C₁-C₅ alkyl, C₁-C₄ alkyl, C₁-C₃ alkyl, C₁-C₂ alkyl, C₂-C₆ alkyl, C₃-C₆ alkyl and C₄-C₆ alkyl. Representative alkyl groups include, but are not limited to, methyl, ethyl, -propyl, 1-methyl ethyl, -butyl, -pentyl, 1,1-dimethyl ethyl, 3-methylhexyl, 2-methylhexyl, 1-ethyl-propyl, and the like. In some embodiments, the alkyl is methyl or ethyl. In some embodiments, the alkyl is —CH(CH₃)₂ or —C(CH₃)₃. Unless stated otherwise specifically in the specification, an alkyl group may be optionally substituted. “Alkylene” or “alkylene chain” refers to a straight or branched divalent hydrocarbon chain linking the rest of the molecule to a radical group. In some embodiments, the alkylene is —CH₂—, —CH₂CH₂—, or —CH₂CH₂CH₂—. In some embodiments, the alkylene is —CH₂—. In some embodiments, the alkylene is —CH₂CH₂—. In some embodiments, the alkylene is —CH₂CH₂CH₂—. Unless stated otherwise specifically in the specification, an alkylene group may be optionally substituted.

The term “alkenylene” or “alkenylene chain” refers to a straight or branched divalent hydrocarbon chain in which at least one carbon-carbon double bond is present linking the rest of the molecule to a radical group. In some embodiments, the alkenylene is —CH═CH—, —CH₂CH═CH— or —CH═CHCH₂—. In some embodiments, the alkenylene is —CH═CH—. In some embodiments, the alkenylene is —CH₂CH═CH—. In some embodiments, the alkenylene is —CH═CHCH₂—.

The term “alkynyl” refers to a type of alkyl group in which at least one carbon-carbon triple bond is present. In one embodiment, an alkenyl group has the formula —C≡C—R^{X}, wherein R^{x} refers to the remaining portions of the alkynyl group. In some embodiments, R^{x} is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃, —C≡CCH₂CH, and —CH₂C≡CH.

The term “aryl” refers to a radical comprising at least one aromatic ring wherein each of the atoms forming the ring is a carbon atom. Aryl groups can be optionally substituted. Examples of aryl groups include, but are not limited to phenyl, and naphthyl. In some embodiments, the aryl is phenyl. Depending on the structure, an aryl group can be a monoradical or a diradical (i.e., an arylene group). Unless stated otherwise specifically in the specification, the term “aryl” or the prefix “ar-” (such as in “aralkyl”) is meant to include aryl radicals that are optionally substituted. In some embodiments, an aryl group comprises a partially reduced cycloalkyl group defined herein (e.g., 1,2-dihydronaphthalene). In some embodiments, an aryl group comprises a fully reduced cycloalkyl group defined herein (e.g., 1,2,3,4-tetrahydronaphthalene). When aryl comprises a cycloalkyl group, the aryl is bonded to the rest of the molecule through an aromatic ring carbon atom. An aryl radical can be a monocyclic or polycyclic (e.g., bicyclic, tricyclic, or tetracyclic) ring system, which may include fused, spiro or bridged ring systems.

The term “cycloalkyl” refers to a monocyclic or polycyclic non-aromatic radical, wherein each of the atoms forming the ring (i.e. skeletal atoms) is a carbon atom. In some embodiments, cycloalkyls are saturated or partially unsaturated. In some embodiments, cycloalkyls are spirocyclic or bridged compounds. In some embodiments, cycloalkyls are fused with an aromatic ring (in which case the cycloalkyl is bonded through a non-aromatic ring carbon atom). Cycloalkyl groups include groups having from 3 to 10 ring atoms. Representative cycloalkyls include, but are not limited to, cycloalkyls having from three to ten carbon atoms, from three to eight carbon atoms, from three to six carbon atoms, or from three to five carbon atoms. Monocyclic cycloalkyl radicals include, for example, cyclopropyl, cyclobutyl, cyclopentyl, cyclohexyl, cycloheptyl, and cyclooctyl. In some embodiments, the monocyclic cycloalkyl is cyclopentyl. In some embodiments, the monocyclic cycloalkyl is cyclopentenyl or cyclohexenyl. In some embodiments, the monocyclic cycloalkyl is cyclopentenyl. Polycyclic radicals include, for example, adamantyl, 1,2-dihydronaphthalenyl, 1,4-dihydronaphthalenyl, tetrainyl, decalinyl, 3,4-dihydronaphthalenyl-1(2H)-one, spiro[2.2]pentyl, norbornyl and bicycle[1.1.1]pentyl. Unless otherwise stated specifically in the specification, a cycloalkyl group may be optionally substituted.

The term “heteroalkylene” or “heteroalkylene chain” refers to a straight or branched divalent heteroalkyl chain linking the rest of the molecule to a radical group. Unless stated otherwise specifically in the specification, the heteroalkyl or heteroalkylene group may be optionally substituted as described below. Representative heteroalkylene groups include, but are not limited to —CH₂—O—CH₂—, —CH₂—N(alkyl)-CH₂—, —CH₂—N(aryl)-CH₂—, —OCH₂CH₂O—, —OCH₂CH₂OCH₂CH₂O—, or —OCH₂CH₂OCH₂CH₂OCH₂CH₂O—.

The term “heteocycloalkyl” refers to a cycloalkyl group that includes at least one heteroatom selected from nitrogen, oxygen, and sulfur. Unless stated otherwise specifically in the specification, the heterocycloalkyl radical may be a monocyclic, or bicyclic ring system, which may include fused (when fused with an aryl or a heteroaryl ring, the heterocycloalkyl is bonded through a non-aromatic ring atom) or bridged ring systems. The nitrogen, carbon or sulfur atoms in the heterocyclyl radical may be optionally oxidized. The nitrogen atom may be optionally quatemized. The heterocycloalkyl radical is partially or fully saturated. Examples of heterocycloalkyl radicals include, but are not limited to, dioxolanyl, thienyl[1,3]dithianyl, tetrahydroquinolyl, tetrahydroisoquinolyl, decahydroquinolyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl. The term heterocycloalkyl also includes all ring forms of carbohydrates, including but not limited to monosaccharides, disaccharides and oligosaccharides. Unless otherwise noted, heterocycloalkyls have from 2 to 12 carbons in the ring. In some embodiments, heterocycloalkyls have from 2 to 10 carbons in the ring. In some embodiments, heterocycloalkyls have from 2 to 10 carbons in the ring and 1 or 2 N atoms. In some embodiments, heterocycloalkyls have from 2 to 10 carbons in the ring and 3 or 4 N atoms. In some embodiments, heterocycloalkyls have from 2 to 12 carbons, 0-2 N atoms, 0-2 O atoms, 0-2 P atoms, and 0-1 S atoms in the ring. In some embodiments, heterocycloalkyls have from 2 to 12 carbons, 1-3 N atoms, 0-1 O atoms, and 0-1 S atoms in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” refers to an aryl group that includes one or more ring heteroatoms selected from nitrogen, oxygen, and sulfur. In some embodiments, heteroaryl is monocyclic or bicyclic. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, furazanyl, indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. Illustrative examples of monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Illustrative examples of bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, heteroaryl is pyridinyl, pyrazinyl, pyrimidinyl, thiazolyl, thienyl, thiadiazolyl or furyl. In some embodiments, a heteroaryl contains 0-6 N atoms in the ring. In some embodiments, a heteroaryl contains 1-4 N atoms in the ring. In some embodiments, a heteroaryl contains 4-6 N atoms in the ring. In some embodiments, a heteroaryl contains 0-4 N atoms, 0-1 O atoms, 0-1 P atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉ heteroaryl. In some embodiments, monocyclic heteroaryl is a C₁-C₅ heteroaryl. In some embodiments, monocyclic heteroaryl is a 5-membered or 6-membered heteroaryl. In some embodiments, a bicyclic heteroaryl is a C₆-C₉ heteroaryl. In some embodiments, a heteroaryl group comprises a partially reduced cycloalkyl or heterocycloalkyl group defined herein (e.g., 7,8-dihydroquinoline). In some embodiments, a heteroaryl group comprises a fully reduced cycloalkyl or heterocycloalkyl group defined herein (e.g., 5,6,7, 8-tetrahydroquinoline). When heteroaryl comprises a cycloalkyl or heterocycloalkyl group, the heteroaryl is bonded to the rest of the molecule through a heteroaromatic ring carbon or hetero atom. A heteroaryl radical can be a monocyclic or polycyclic (e.g., bicyclic, tricyclic, or tetracyclic) ring system, which may include fused, spiro or bridged ring systems.

The term “optionally substituted” or “substituted” means that the referenced group is optionally substituted with one or more additional group(s) individually and independently selected from D, halogen, —CN, —NH2, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —NH(cyclopropyl), —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

As used herein, “AJICAP™ technology,” “AJICAP™ methods,” and similar terms refer to systems and methods (currently produced by Ajinomoto Bio-Pharma Services (“Ajinomoto”)) for the site specific functionalization of antibodies and related molecules using affinity peptides to deliver the desired functionalization to the desired site. General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. In some embodiments, such methodologies site specifically incorporate the desired functionalization at lysine residues at a position selected from position 246, position 248, position 288, position 290, and position 317 of an antibody Fc region (e.g., an IgG1 Fc region) (EU numbering). In some embodiments, the desired functionalization is incorporated at residue position 248 of an antibody Fc region (EU numbering). In some embodiments, position 248 corresponds to the 18^{th} residue in a human IgG CH2 region (EU numbering).

“Composition AA” refers to an IL-7 polypeptide having an amino acid sequences as set forth in SEQ ID NO: 3 with an N-terminal modification having a structure of

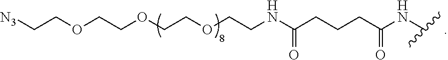

In the structure above, the N attached to the squiggly line is the N-terminal amino group of the IL-7.

“SEQ ID NOs: 46-47” refers to the unmodified anti-PD-1 antibody Pembrolizumab as set forth in Table 1.

“SEQ ID NOs: 76-77” refers to the unmodified anti-PD-1 antibody LZM-009 as set forth in Table 1.

“Composition A” refers to an anti-PD-1 antibody/IL-7 conjugate prepared form a reaction of Composition AA and anti-PD-1 antibody SEQ. ID. NO 46-47. Composition A is formed from a reaction of the azide functionality of Composition AA with a DBCO functionality attached to residue K248 of the Fc region of Pembrolizumab (Eu numbering). The DBCO functionality is added to Pembrolizumab using an affinity peptide system according to AJICAP™ technology from Ajinomoto. Composition A has a drug-antibody ratio of 1.

“Composition B” refers to an anti-PD-1 antibody/IL-7 conjugate prepared form a reaction of Composition AA and anti-PD-1 antibody SEQ. ID. NO 46-47. Composition B is formed from a reaction of the azide functionality of Composition AA. This is conjugated to a DBCO functionality attached to residue K248 of the Fc region of Pembrolizumab (Eu numbering). The DBCO functionality is added to Pembrolizumab using an affinity peptide system according to AJICAP™ technology from Ajinomoto. Composition B has a drug-antibody ratio of 2.

“Composition C” refers to an anti-PD-1 antibody/IL-7 conjugate prepared form a reaction of Composition AA and anti-PD-1 antibody SEQ. ID. NO 76-77. Composition C is formed from a reaction of the azide functionality of Composition AA where an N-terminal conjugation handle has been added. Composition AA is conjugated to a DBCO functionality attached to residue K248 of the Fc region of LZM-009 (Eu numbering). The DBCO functionality is added to LZM-009 using an affinity peptide system according to AJICAP™ technology from Ajinomoto. Composition C has a drug-antibody ratio of 1.

Anti-PD-1 Polypeptides Conjugated to Cytokines such as IL-7

Programmed cell death protein 1 (also known as PD-1 and CD279), is a cell surface receptor that plays a role in down-regulating the immune system and promoting self-tolerance by suppressing T cell inflammatory activity. PD-1 is an immune cell inhibitory molecule that is expressed on activated B cells, T cells and myeloid cells. PD-1 represents an immune checkpoint and guards against autoimmunity via a dual mechanism of promoting apoptosis (programmed cell death) in antigen-specific T-cells in lymph nodes while reducing apoptosis in regulatory T cells. PD-1 is a member of the CD28/CTLA-4/ICOS costimulatory receptor family that delivers negative signals that affect primarily T and B cell immunity. PD-1 is monomeric both in solution as well as on cell surface, in contrast to CTLA-4 and other family members that are all disulfide-linked homodimers. Signaling through the PD-1 inhibitory receptor upon binding its ligand, PD-L1, suppresses immune responses against autoantigens and tumors and plays a role in the maintenance of peripheral immune tolerance. The interaction between PD-1 and PD-L1 results in a decrease in tumor infiltrating lymphocytes, a decrease in T cell receptor mediated proliferation, and immune evasion by the cancerous cells. A non-limiting, exemplary, human PD-1 amino acid sequence is MQIPQAPWPVVWAVLQLGWRPGWFLDSPDRPWNPPTFSPALLVVTEGDNATFTCSFSN TSESFVLNWYRMSPSNQTDKLAAFPEDRSQPGQDCRFRVTQLPNGRDFHMSVVRARRN DSGTYLCGAISLAPKAQIKESLRAELRVTERRAEVPTAHPSPSPRPAGQFQTLVVGVVGG LLGSLVLLVWVLAVICSRAARGTIGARRTGQPLKEDPSAVPVFSVDYGELDFQWREKTP EPPVPCVPEQTEYATIVFPSGMGTSSPARRGSADGPRSAQPLRPEDGHCSWPL (SEQ ID NO: 120).

Provided herein are polypeptides, such as antibodies and anti-PD-1 antigen binding fragments, which bind to programmed cell death protein 1 (PD-1) which are conjugated to one or more cytokine molecules or derivatives thereof. The conjugates provided herein are effective for simultaneously delivering the cytokine and the polypeptide which selectively binds to PD-1 to a target cell. This simultaneous delivery of both agents to the same cell has numerous benefits, including improved IL-7 polypeptide selectivity, enhanced the therapeutic potential of IL-7, and minimized risk of side effects from administering IL-7 therapies.

The conjugate compositions provided herein utilize linkers to attach the polypeptides which bind to PD-1 to the cytokines, such as IL-7 polypeptides and derivatives thereof. In some embodiments, the linkers are attached to each moiety the polypeptide which selectively binds to PD-1 and the cytokine at specific residues or a specific subset of residues. In some embodiments, the linkers are attached to each moiety in a site-selective manner, such that a population of the conjugate is substantially uniform. This can be accomplished in a variety of ways as provided herein, including by site-selectively adding reagents for a conjugation reaction to a moiety to be conjugated, synthesizing, or otherwise preparing a moiety to be conjugated with a desired reagent for a conjugation reaction, or a combination of these two approaches. Using these approaches, the sites of attachment (such as specific amino acid residues) of the linker to each moiety can be selected with precision. Additionally, these approaches allow a variety of linkers to be employed for the composition which are not limited to amino acid residues as is required for fusion proteins. This combination of linker choice and precision attachment to the moieties allows the linker to also, in some embodiments, perform the function of modulating the activity of one of the moieties, for example if the linker is attached to the cytokine at a position that interacts with a receptor of the cytokine.

### Anti-PD-1 Polypeptides

In some embodiments, an anti-PD-1 polypeptide of the disclosure specifically binds to PD-1. An anti-PD-1 polypeptide selectively binds or preferentially binds to a target if it binds with greater affinity, avidity, more readily, and/or with greater duration than it binds to other substances. As such, “specific binding” or “preferential binding” does not necessarily require (although it can include) exclusive binding. Generally, but not necessarily, reference to specific binding means preferential binding where the affinity of the antibody, or antigen binding fragment thereof, is at least at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater than the affinity of the antibody for unrelated amino acid sequences. An anti-PD-1 polypeptide or an anti-PD-1 antigen binding fragment of the disclosure can block interaction of PD-1 with a ligand (e.g., PD-L1).

As used herein, the term “antibody” refers to an immunoglobulin (Ig), polypeptide, or a protein having a binding domain which is, or is homologous to, an antigen binding domain. The term further includes “antigen binding fragments” and other interchangeable terms for similar binding fragments as described below. Native antibodies and native immunoglobulins (Igs) are generally heterotetrameric glycoproteins of about 150,000 Daltons, composed of two identical light chains and two identical heavy chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (“VH”) followed by a number of constant domains (“CH”). Each light chain has a variable domain at one end (“VL”) and a constant domain (“CL”) at its other end; the constant domain of the light chain is aligned with the first constant domain of the heavy chain, and the light-chain variable domain is aligned with the variable domain of the heavy chain. Particular amino acid residues are believed to form an interface between the light- and heavy-chain variable domains.

In some instances, an antibody or an antigen binding fragment comprises an isolated antibody or antigen binding fragment, a purified antibody or antigen binding fragment, a recombinant antibody or antigen binding fragment, a modified antibody or antigen binding fragment, or a synthetic antibody or antigen binding fragment.

Antibodies and antigen binding fragments herein can be partly or wholly synthetically produced. An antibody or antigen binding fragment can be a polypeptide or protein having a binding domain which can be, or can be homologous to, an antigen binding domain. In one instance, an antibody or an antigen binding fragment can be produced in an appropriate in vivo animal model and then isolated and/or purified.

Depending on the amino acid sequence of the constant domain of its heavy chains, immunoglobulins (Igs) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. An Ig or portion thereof can, in some cases, be a human Ig. In some instances, a C_{H}3 domain can be from an immunoglobulin. In some cases, a chain or a part of an antibody or antigen binding fragment, a modified antibody or antigen binding fragment, or a binding agent can be from an Ig. In such cases, an Ig can be IgG, an IgA, an IgD, an IgE, or an IgM, or is derived therefrom. In cases where the Ig is an IgG, it can be a subtype of IgG, wherein subtypes of IgG can include IgG1, an IgG2a, an IgG2b, an IgG3, or an IgG4. In some cases, a C_{H}3 domain can be from an immunoglobulin selected from the group consisting of an IgG, an IgA, an IgD, an IgE, and an IgM, or derived therefrom. In some embodiments, an antibody or antigen binding fragment described herein comprises an IgG or is derived therefrom. In some instances, an antibody or antigen binding fragment comprises an IgG1 or is derived therefrom. In some instances, an antibody or antigen binding fragment comprises an IgG4 or is derived therefrom. In some embodiments, an antibody or antigen binding fragment described herein comprises an IgM, is derived therefrom, or is a monomeric form of IgM. In some embodiments, an antibody or antigen binding fragment described herein comprises an IgE or is derived therefrom. In some embodiments, an antibody or antigen binding fragment described herein comprises an IgD or is derived therefrom. In some embodiments, an antibody or antigen binding fragment described herein comprises an IgA or is derived therefrom.

The “light chains” of antibodies (immunoglobulins) from any vertebrate species can be assigned to one of two clearly distinct types, called kappa (“κ” or “K”) or lambda (“λ”), based on the amino acid sequences of their constant domains.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (e.g., Kabat et al., Sequences of Proteins of Immunological Interest, (5th Ed., 1991, National Institutes of Health, Bethesda Md. (1991), pages 647-669; hereafter “Kabat”); and (2) an approach based on crystallographic studies of antigen-antibody complexes (Al-Iazikani et al. (1997) J. Molec. Biol. 273:927-948)). As used herein, a CDR may refer to CDRs defined by either approach or by a combination of both approaches.

With respect to antibodies, the term “variable domain” refers to the variable domains of antibodies that are used in the binding and specificity of each particular antibody for its particular antigen. However, the variability is not evenly distributed throughout the variable domains of antibodies. Rather, it is concentrated in three segments called hypervariable regions (also known as CDRs) in both the light chain and the heavy chain variable domains. More highly conserved portions of variable domains are called the “framework regions” or “FRs.” The variable domains of unmodified heavy and light chains each contain four FRs (FR1, FR2, FR3, and FR4), largely adopting a β-sheet configuration interspersed with three CDRs which form loops connecting and, in some cases, part of the j-sheet structure. The CDRs in each chain are held together in close proximity by the FRs and, with the CDRs from the other chain, contribute to the formation of the antigen binding site of antibodies (see, Kabat).

The terms “hypervariable region” and “CDR” when used herein, refer to the amino acid residues of an antibody which are responsible for antigen binding. The CDRs comprise amino acid residues from three sequence regions which bind in a complementary manner to an antigen and are known as CDR1, CDR2, and CDR3 for each of the V_{H} and V_{L} chains. In the light chain variable domain, the CDRs typically correspond to approximately residues 24-34 (CDRL1), 50-56 (CDRL2), and 89-97 (CDRL3), and in the heavy chain variable domain the CDRs typically correspond to approximately residues 31-35 (CDRH1), 50-65 (CDRH2), and 95-102 (CDRH3) according to Kabat. It is understood that the CDRs of different antibodies may contain insertions, thus the amino acid numbering may differ. The Kabat numbering system accounts for such insertions with a numbering scheme that utilizes letters attached to specific residues (e.g., 27A, 27B, 27C, 27D, 27E, and 27F of CDRL1 in the light chain) to reflect any insertions in the numberings between different antibodies. Alternatively, in the light chain variable domain, the CDRs typically correspond to approximately residues 26-32 (CDRL1), 50-52 (CDRL2), and 91-96 (CDRL3), and in the heavy chain variable domain, the CDRs typically correspond to approximately residues 26-32 (CDRH1), 53-55 (CDRH2), and 96-101 (CDRH3) according to Chothia and Lesk (J. Mol. Biol., 196: 901-917 (1987)).

As used herein, “framework region,” “FW,” or “FR” refers to framework amino acid residues that form a part of the antigen binding pocket or groove. In some embodiments, the framework residues form a loop that is a part of the antigen binding pocket or groove and the amino acids residues in the loop may or may not contact the antigen. Framework regions generally comprise the regions between the CDRs. In the light chain variable domain, the FRs typically correspond to approximately residues 0-23 (FRL1), 35-49 (FRL2), 57-88 (FRL3), and 98-109 and in the heavy chain variable domain the FRs typically correspond to approximately residues 0-30 (FRH1), 36-49 (FRH2), 66-94 (FRH3), and 103-133 according to Kabat. As discussed above with the Kabat numbering for the light chain, the heavy chain too accounts for insertions in a similar manner (e.g., 35A, 35B of CDRH1 in the heavy chain). Alternatively, in the light chain variable domain, the FRs typically correspond to approximately residues 0-25 (FRL1), 33-49 (FRL2) 53-90 (FRL3), and 97-109 (FRL4), and in the heavy chain variable domain, the FRs typically correspond to approximately residues 0-25 (FRH1), 33-52 (FRH2), 56-95 (FRH3), and 102-113 (FRH4) according to Chothia and Lesk, Id. The loop amino acids of a FR can be assessed and determined by inspection of the three-dimensional structure of an antibody heavy chain and/or antibody light chain. The three-dimensional structure can be analyzed for solvent accessible amino acid positions as such positions are likely to form a loop and/or provide antigen contact in an antibody variable domain. Some of the solvent accessible positions can tolerate amino acid sequence diversity and others (e.g., structural positions) are, generally, less diversified. The three-dimensional structure of the antibody variable domain can be derived from a crystal structure or protein modeling.

In the present disclosure, the following abbreviations (in the parentheses) are used in accordance with the customs, as necessary: heavy chain (H chain), light chain (L chain), heavy chain variable region (VH), light chain variable region (VL), complementarity determining region (CDR), first complementarity determining region (CDR1), second complementarity determining region (CDR2), third complementarity determining region (CDR3), heavy chain first complementarity determining region (VH CDR1), heavy chain second complementarity determining region (VH CDR2), heavy chain third complementarity determining region (VH CDR3), light chain first complementarity determining region (VL CDR1), light chain second complementarity determining region (VL CDR2), and light chain third complementarity determining region (VL CDR3).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is generally defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, C_{H}2 and C_{H}3.

“Antibodies” useful in the present disclosure encompass, but are not limited to, monoclonal antibodies, polyclonal antibodies, chimeric antibodies, bispecific antibodies, multispecific antibodies, heteroconjugate antibodies, humanized antibodies, human antibodies, grafted antibodies, deimmunized antibodies, mutants thereof, fusions thereof, immunoconjugates thereof, antigen binding fragments thereof, and/or any other modified configuration of the immunoglobulin molecule that comprises an antigen recognition site of the required specificity, including glycosylation variants of antibodies, amino acid sequence variants of antibodies, and covalently modified antibodies. In certain embodiments of the methods and conjugates provided herein, the antibody requires an Fc region to enable attachment of a linker between the antibody and the protein (e.g., attachment of the linker using an affinity peptide, such as in AJICAP™ technology).

In some instances, an antibody is a monoclonal antibody. As used herein, a “monoclonal antibody” refers to an antibody obtained from a population of substantially homogeneous antibodies, i.e., the individual antibodies comprising the population are identical except for possible naturally-occurring mutations that may be present in minor amounts. In contrast to polyclonal antibody preparations, which typically include different antibodies directed against different determinants (epitopes), each monoclonal antibody is directed against a single determinant on the antigen (epitope). The modifier “monoclonal” indicates the character of the antibody as being obtained from a substantially homogeneous population of antibodies and is not to be construed as requiring production of the antibody by any particular method.

In some instances, an antibody is a humanized antibody. As used herein, “humanized” antibodies refer to forms of non-human (e.g., murine) antibodies that are specific chimeric immunoglobulins, immunoglobulin chains, or fragments thereof that contain minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and biological activity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, a humanized antibody comprises substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in, for example, WO 99/58572. Other forms of humanized antibodies have one or more CDRs (one, two, three, four, five, or six) which are altered with respect to the original antibody, which are also termed one or more CDRs “derived from” one or more CDRs from the original antibody.

If needed, an antibody or an antigen binding fragment described herein can be assessed for immunogenicity and, as needed, be deimmunized (i.e., the antibody is made less immunoreactive by altering one or more T cell epitopes). As used herein, a “deimmunized antibody” means that one or more T cell epitopes in an antibody sequence have been modified such that a T cell response after administration of the antibody to a subject is reduced compared to an antibody that has not been deimmunized. Analysis of immunogenicity and T-cell epitopes present in the antibodies and antigen binding fragments described herein can be carried out via the use of software and specific databases. Exemplary software and databases include iTope™ developed by Antitope of Cambridge, England. iTope™, is an in silico technology for analysis of peptide binding to human MHC class II alleles. The iTope™ software predicts peptide binding to human MHC class II alleles and thereby provides an initial screen for the location of such “potential T cell epitopes.” iTope™ software predicts favorable interactions between amino acid side chains of a peptide and specific binding pockets within the binding grooves of 34 human MHC class II alleles. The location of key binding residues is achieved by the in silico generation of 9mer peptides that overlap by one amino acid spanning the test antibody variable region sequence. Each 9mer peptide can be tested against each of the 34 MHC class II allotypes and scored based on their potential “fit” and interactions with the MHC class II binding groove. Peptides that produce a high mean binding score (>0.55 in the iTope™ scoring function) against >50% of the MHC class II alleles are considered as potential T cell epitopes. In such regions, the core 9 amino acid sequence for peptide binding within the MHC class II groove is analyzed to determine the MHC class II pocket residues (P1, P4, P6, P7, and P9) and the possible T cell receptor (TCR) contact residues (P-1, P2, P3, P5, P8). After identification of any T-cell epitopes, amino acid residue changes, substitutions, additions, and/or deletions can be introduced to remove the identified T-cell epitope. Such changes can be made so as to preserve antibody structure and function while still removing the identified epitope. Exemplary changes can include, but are not limited to, conservative amino acid changes.

An antibody can be a human antibody. As used herein, a “human antibody” means an antibody having an amino acid sequence corresponding to that of an antibody produced by a human and/or that has been made using any suitable technique for making human antibodies. This definition of a human antibody includes antibodies comprising at least one human heavy chain polypeptide or at least one human light chain polypeptide. One such example is an antibody comprising murine light chain and human heavy chain polypeptides. In one embodiment, the human antibody is selected from a phage library, where that phage library expresses human antibodies. Human antibodies can also be made by introducing human immunoglobulin loci into transgenic animals, e.g., mice in which the endogenous immunoglobulin genes have been partially or completely inactivated. Alternatively, the human antibody may be prepared by immortalizing human B lymphocytes that produce an antibody directed against a target antigen (such B lymphocytes may be recovered from an individual or may have been immunized in vitro).

Any of the antibodies herein can be bispecific. Bispecific antibodies are antibodies that have binding specificities for at least two different antigens and can be prepared using the antibodies disclosed herein. Traditionally, the recombinant production of bispecific antibodies was based on the coexpression of two immunoglobulin heavy chain-light chain pairs, with the two heavy chains having different specificities. Bispecific antibodies can be composed of a hybrid immunoglobulin heavy chain with a first binding specificity in one arm, and a hybrid immunoglobulin heavy chain-light chain pair (providing a second binding specificity) in the other arm. This asymmetric structure, with an immunoglobulin light chain in only one half of the bispecific molecule, facilitates the separation of the desired bispecific compound from unwanted immunoglobulin chain combinations.

According to one approach to making bispecific antibodies, antibody variable domains with the desired binding specificities (antibody-antigen combining sites) are fused to immunoglobulin constant domain sequences. The fusion can be with an immunoglobulin heavy chain constant domain, comprising at least part of the hinge, CH2 and CH3 regions. The first heavy chain constant region (CH1), containing the site necessary for light chain binding, can be present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

In some instances, an antibody herein is a chimeric antibody. “Chimeric” forms of non-human (e.g., murine) antibodies include chimeric antibodies which contain minimal sequence derived from a non-human Ig. For the most part, chimeric antibodies are murine antibodies in which at least a portion of an immunoglobulin constant region (Fc), typically that of a human immunoglobulin, is inserted in place of the murine Fc. Chimeric or hybrid antibodies also may be prepared in vitro using suitable methods of synthetic protein chemistry, including those involving cross-linking agents. For example, immunotoxins may be constructed using a disulfide exchange reaction or by forming a thioether bond. Examples of suitable reagents for this purpose include iminothiolate and methyl-4-mercaptobutyrimidate.

Provided herein are antibodies and antigen binding fragments thereof, modified antibodies and antigen binding fragments thereof, and binding agents that specifically bind to one or more epitopes on one or more target antigens. In one instance, a binding agent selectively binds to an epitope on a single antigen. In another instance, a binding agent is bivalent and either selectively binds to two distinct epitopes on a single antigen or binds to two distinct epitopes on two distinct antigens. In another instance, a binding agent is multivalent (i.e., trivalent, quatravalent, etc.) and the binding agent binds to three or more distinct epitopes on a single antigen or binds to three or more distinct epitopes on two or more (multiple) antigens.

Antigen binding fragments of any of the antibodies herein are also contemplated. The terms “antigen binding portion of an antibody,” “antigen binding fragment,” “antigen binding domain,” “antibody fragment,” or a “functional fragment of an antibody” are used interchangeably herein to refer to one or more fragments of an antibody that retain the ability to specifically bind to an antigen. Representative antigen binding fragments include, but are not limited to, a Fab, a Fab′, a F(ab′)₂, a bispecific F(ab′)₂, a trispecific F(ab′)₂, a variable fragment (Fv), a single chain variable fragment (scFv), a dsFv, a bispecific scFv, a variable heavy domain, a variable light domain, a variable NAR domain, bispecific scFv, an AVIMER®, a minibody, a diabody, a bispecific diabody, triabody, a tetrabody, a minibody, a maxibody, a camelid, a VHH, a minibody, an intrabody, fusion proteins comprising an antibody portion (e.g., a domain antibody), a single chain binding polypeptide, a scFv-Fc, a Fab-Fc, a bispecific T cell engager (BiTE; two scFvs produced as a single polypeptide chain, where each scFv comprises an amino acid sequences a combination of CDRs or a combination of VL/VL described herein), a tetravalent tandem diabody (TandAb; an antibody fragment that is produced as a non-covalent homodimer folder in a head-to-tail arrangement, e.g., a TandAb comprising an scFv, where the scFv comprises an amino acid sequences a combination of CDRs or a combination of VL/VL described herein), a Dual-Affinity Re-targeting Antibody (DART; different scFvs joined by a stabilizing interchain disulphide bond), a bispecific antibody (bscAb; two single-chain Fv fragments joined via a glycine-serine linker), a single domain antibody (sdAb), a fusion protein, a bispecific disulfide-stabilized Fv antibody fragment (dsFv-dsFv′; two different disulfide-stabilized Fv antibody fragments connected by flexible linker peptides). In certain embodiments of the invention, a full length antibody (e.g., an antigen binding fragment and an Fc region) are preferred.

Heteroconjugate polypeptides comprising two covalently joined antibodies or antigen binding fragments of antibodies are also within the scope of the disclosure. Suitable linkers may be used to multimerize binding agents. Non-limiting examples of linking peptides include, but are not limited to, (GS)ₙ (SEQ ID NO: 24), (GGS)ₙ (SEQ ID NO: 25), (GGGS)ₙ (SEQ ID NO: 26), (GGSG)ₙ (SEQ ID NO: 27), or (GGSGG)ₙ (SEQ ID NO: 28), (GGGGS)ₙ (SEQ ID NO: 29), wherein n is 1, 2, 3, 4, 5, 6, 7, 8, 9, or 10. For example, a linking peptide can be (GGGGS)₃ (SEQ ID NO: 30) or (GGGGS)₄ (SEQ ID NO: 31). In some embodiments, a linking peptide bridges approximately 3.5 nm between the carboxy terminus of one variable region and the amino terminus of the other variable region. Linkers of other sequences have been designed and used. Linkers can in turn be modified for additional functions, such as attachment of drugs or attachment to solid supports.

As used herein, the term “avidity” refers to the resistance of a complex of two or more agents to dissociation after dilution. Apparent affinities can be determined by methods such as an enzyme-linked immunosorbent assay (ELISA) or any other suitable technique. Avidities can be determined by methods such as a Scatchard analysis or any other suitable technique.

As used herein, the term “affinity” refers to the equilibrium constant for the reversible binding of two agents and is expressed as K_{D}. The binding affinity (K_{D}) of an antibody or antigen binding fragment herein can be less than 500 nM, 475 nM, 450 nM, 425 nM, 400 nM, 375 nM, 350 nM, 325 nM, 300 nM, 275 nM, 250 nM, 225 nM, 200 nM, 175 nM, 150 nM, 125 nM, 100 nM, 90 nM, 80 nM, 70 nM, 50 nM, 50 nM, 49 nM, 48 nM, 47 nM, 46 nM, 45 nM, 44 nM, 43 nM, 42 nM, 41 nM, 40 nM, 39 nM, 38 nM, 37 nM, 36 nM, 35 nM, 34 nM, 33 nM, 32 nM, 31 nM, 30 nM, 29 nM, 28 nM, 27 nM, 26 nM, 25 nM, 24 nM, 23 nM, 22 nM, 21 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, 1 nM, 990 pM, 980 pM, 970 pM, 960 pM, 950 pM, 940 pM, 930 pM, 920 pM, 910 pM, 900 pM, 890 pM, 880 pM, 870 pM, 860 pM, 850 pM, 840 pM, 830 pM, 820 pM, 810 pM, 800 pM, 790 pM, 780 pM, 770 pM, 760 pM, 750 pM, 740 pM, 730 pM, 720 pM, 710 pM, 700 pM, 690 pM, 680 pM, 670 pM, 660 pM, 650 pM, 640 pM, 630 pM, 620 pM, 610 pM, 600 pM, 590 pM, 580 pM, 570 pM, 560 pM, 550 pM, 540 pM, 530 pM, 520 pM, 510 pM, 500 pM, 490 pM, 480 pM, 470 pM, 460 pM, 450 pM, 440 pM, 430 pM, 420 pM, 410 pM, 400 pM, 390 pM, 380 pM, 370 pM, 360 pM, 350 pM, 340 pM, 330 pM, 320 pM, 310 pM, 300 pM, 290 pM, 280 pM, 270 pM, 260 pM, 250 pM, 240 pM, 230 pM, 220 pM, 210 pM, 200 pM, 190 pM, 180 pM, 170 pM, or any integer therebetween. Binding affinity may be determined using surface plasmon resonance (SPR), KINEXA® Biosensor, scintillation proximity assays, enzyme linked immunosorbent assay (ELISA), ORIGEN immunoassay (IGEN), fluorescence quenching, fluorescence transfer, yeast display, or any combination thereof. Binding affinity may also be screened using a suitable bioassay.

As used herein, the term “avidity” refers to the resistance of a complex of two or more agents to dissociation after dilution. Apparent affinities can be determined by methods such as an enzyme linked immunosorbent assay (ELISA) or any other technique familiar to one of skill in the art. Avidities can be determined by methods such as a Scatchard analysis or any other technique familiar to one of skill in the art.

Also provided herein are affinity matured antibodies. The following methods may be used for adjusting the affinity of an antibody and for characterizing a CDR. One way of characterizing a CDR of an antibody and/or altering (such as improving) the binding affinity of a polypeptide, such as an antibody, is termed “library scanning mutagenesis.” Generally, library scanning mutagenesis works as follows. One or more amino acid position in the CDR is replaced with two or more (such as 3, 4, 5, 6, 7, 8, 9, 10, 11, 12, 13, 14, 15, 16, 17, 18, 19, or 20) amino acids. This generates small libraries of clones (in some embodiments, one for every amino acid position that is analyzed), each with a complexity of two or more members (if two or more amino acids are substituted at every position). Generally, the library also includes a clone comprising the native (unsubstituted) amino acid. A small number of clones, for example, about 20-80 clones (depending on the complexity of the library), from each library can be screened for binding specificity or affinity to the target polypeptide (or other binding target), and candidates with increased, the same, decreased, or no binding are identified. Binding affinity may be determined using Biacore surface plasmon resonance analysis, which detects differences in binding affinity of about 2-fold or greater.

In some instances, an antibody or antigen binding fragment is bispecific or multispecific and can specifically bind to more than one antigen. In some cases, such a bispecific or multispecific antibody or antigen binding fragment can specifically bind to 2 or more different antigens. In some cases, a bispecific antibody or antigen binding fragment can be a bivalent antibody or antigen binding fragment. In some cases, a multi specific antibody or antigen binding fragment can be a bivalent antibody or antigen binding fragment, a trivalent antibody or antigen binding fragment, or a quatravalent antibody or antigen binding fragment.

An antibody or antigen binding fragment described herein can be isolated, purified, recombinant, or synthetic.

The antibodies described herein may be made by any suitable method. Antibodies can often be produced in large quantities, particularly when utilizing high level expression vectors.

In one embodiment, an anti-PD1 antibody or an anti-PD1 antigen binding fragment of the disclosure comprises a combination of a heavy chain variable region (VH) and a light chain variable region (VL) described herein. In another embodiment, an anti-PD1 antibody or an anti-PD1 antigen binding fragment of the disclosure comprises a combination of complementarity determining regions (VH CDR1, VH CDR2, VH CDR3, VL CDR1, VL CDR2, and VL CDR3) described herein. In one embodiment, an anti-PD-1 antibody or an anti-PD-1 antigen binding fragment of the disclosure comprises a modified Tislelizumab, Baizean, 0KVO411B3N, BGB-A317, hu317-1/IgG4mt2, Sintilimab, Tyvyt, IBI-308, Toripalimab, TeRuiPuLi, Terepril, Tuoyi, JS-001, TAB-001, Camrelizumab, HR-301210, INCSHR-01210, SHR-1210, Cemiplimab, Cemiplimab-rwlc, LIBTAYO®, 6QVL057INT, H4H7798N, REGN-2810, SAR-439684, Lambrolizumab, Pembrolizumab, KEYTRUDA®, MK-3475, SCH-900475, h409A11, Nivolumab, Nivolumab BMS, OPDIVO®, BMS-936558, MDX-1106, ONO-4538, Prolgolimab, Forteca, BCD-100, Penpulimab, AK-105, Zimberelimab, AB-122, GLS-010, WBP-3055, Balstilimab, 1Q2QT5M7EO, AGEN-2034, AGEN-2034w, Genolimzumab, Geptanolimab, APL-501, CBT-501, GB-226, Dostarlimab, ANB-011, GSK-4057190A, P0GVQ9A4S5, TSR-042, WBP-285, Serplulimab, HLX-10, CS-1003, Retifanlimab, 2Y3T5IF0IZ, INCMGA-00012, INCMGA-0012, MGA-012, Sasanlimab, LZZOIC2EWP, PF-06801591, RN-888, Spartalizumab, NVP-LZV-184, PDR-001, QOG25L6Z8Z, Relatlimab/nivolumab, BMS-986213, Cetrelimab, JNJ-3283, JNJ-63723283, LYK98WP91F, Tebotelimab, MGD-013, BCD-217, BAT-1306, HX-008, MEDI-5752, JTX-4014, Cadonilimab, AK-104, BI-754091, Pidilizumab, CT-011, MDV-9300, YBL-006, AMG-256, RG-6279, RO-7284755, BH-2950, IBI-315, RG-6139, RO-7247669, ONO-4685, AK-112, 609-A, LY-3434172, T-3011, MAX-10181, AMG-404, IBI-318, MGD-019, INCB-086550, ONCR-177, LY-3462817, RG-7769, RO-7121661, F-520, XmAb-23104, Pd-1-pik, SG-001, S-95016, Sym-021, LZM-009, Budigalimab, 6VDO4TY300, ABBV-181, PR-1648817, CC-90006, XmAb-20717, 2661380, AMP-224, B7-DCIg, EMB-02, ANB-030, PRS-332, [89Zr]Deferoxamide-Pembrolizumab, 89Zr-Df-Pembrolizumab, [89Zr]Df-Pembrolizumab, STI-1110, STI-A1110, CX-188, mPD-1 Pb-Tx, MCLA-134, 244C8, ENUM 224C8, ENUM C8, 388D4, ENUM 388D4, ENUM D4, MEDIO680, or AMP-514. In some embodiments, the anti-PD-1 polypeptide is modified Pembrolizumab. In some embodiments, the anti-PD-1 polypeptide is modified with mAB3. In some embodiments, the anti-PD-1 polypeptide is modified with mAB4.

In one embodiment, an anti-PD-1 antibody or an anti-PD-1 antigen binding fragment of the disclosure comprises a modified Tislelizumab, Sintilimab, Toripalimab, Terepril, Camrelizumab, Cemiplimab, Pembrolizumab Nivolumab, Prolgolimab, Penpulimab, Zimberelimab, Balstilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, or Budigalimab.

In some embodiments, the anti-PD-1 polypeptide is Nivolumab, Pembrolizumab, LZM-009, Dostarlimab, Sintilimab, Spartalizumab, Tislelizumab, or Cemiplimab. In some embodiment, the anti-PD-1 polypeptide is Dostarlimab, Sintilimab, Spartalizumab, or Tislelizumab. In some embodiments, the anti-PD-1 polypeptide is Nivolumab, Pembrolizumab, LZM-009, or Cemiplimab.

It is contemplated that generic or biosimilar versions of the named antibodies herein which share the same amino acid sequence as the indicated antibodies are also encompassed when the name of the antibody is used. In some embodiments, the anti-PD-1 antibody is a biosimilar of Tislelizumab, Sintilimab, Toripalimab, Terepril, Camrelizumab, Cemiplimab, Pembrolizumab Nivolumab, Prolgolimab, Penpulimab, Zimberelimab, Bastilimab, Genolimzumab, Geptanolimab, Dostarlimab, Serplulimab, Retifanlimab, Sasanlimab, Spartalizumab, Cetrelimab, Tebotelimab, Cadonilimab, A Pidilizumab, LZM-009, orfBudigalimab. In some embodiments, the anti-PD-1 antibody is a biosimilar of any one of the antibodies provided herein.

TABLE 1 provides the sequences of exemplary anti-PD-1 polypeptides (e.g., anti-PD-1 antibodies) and anti-PD-1 antigen binding fragments that can be modified to prepare anti-PD-1 immunoconjugates. TABLE 1 also shows provides combinations of CDRs that can be utilized in a modified anti-PD-1 immunoconjugate. Reference to an anti-PD-1 polypeptide herein may alternatively refer to an anti-PD-1 antigen binding fragment.

TABLE 1

| Antibody or | | |
|---|---|---|
| Ag-binding | | SEQ ID |
| fragment | Sequence | NO |
| Tislelizumab, | QVQLQESGPGLVKPSETLSLTCTVSGFSLTSYGVHWIRQPPG | 32 |
| Baizean, | KGLEWIGVIYADGSTNYNPSLKSRVTISKDTSKNQVSLKLSS | |
| 0KVO411B3N, | VTAADTAVYYCARAYGNYWYIDVWGQGTTVTVSSASTKG | |
| BGB-A317, | PSVFPLAPCSRSTSESTAALGCLVKDYFPEPVTVSWNSGALT | |
| hu317- | SGVHTFPAVLQSSGLYSLSSVVTVPSSSLGTKTYTCNVDHKP | |
| 1/IgG4mt2 | SNTKVDKRVESKYGPPCPPCPAPPVAGGPSVFLFPPKPKDTL | |
| VH | MISRTPEVTCVVVAVSQEDPEVQFNWYVDGVEVHNAKTKP | |
| | REEQFNSTYRVVSVLTVVHQDWLNGKEYKCKVSNKGLPSSI | |
| | EKTISKAKGQPREPQVYTLPPSQEEMTKNQVSLTCLVKGFY | |
| | PSDIAVEWESNGQPENNYKTTPPVLDSDGSFFLYSKLTVDKS | |
| | RWQEGNVFSCSVMHEALHNHYTQKSLSLSLGK | |
| Tislelizumab, | DIVMTQSPDSLAVSLGERATINCKSSESVSNDVAWYQQKPG | 33 |
| Baizean, | QPPKLLINYAFHRFTGVPDRFSGSGYGTDFTLTISSLQAEDV | |
| 0KVO411B3N, | AVYYCHQAYSSPYTFGQGTKLEIKRTVAAPSVFIFPPSDEQL | |
| BGB-A317, | KSGTASVVCLLNNFYPREAKVQWKVDNALQSGNSQESVTE | |
| hu317- | QDSKDSTYSLSSTLTLSKADYEKHKVYACEVTHQGLSSPVT | |
| 1/IgG4mt2 | KSFNRGEC | |
| VL | | |
| Sintilimab, | QVQLVQSGAEVKKPGSSVKVSCKASGGTFSSYAISWVRQAP | 34 |
| Tyvyt, IBI-308 | GQGLEWMGLIIPMFDTAGYAQKFQGRVAITVDESTSTAYM | |
| VH | ELSSLRSEDTAVYYCARAEHSSTGTFDYWGQGTLVTVSSAS | |
| | TKGPSVFPLAPCSRSTSESTAALGCLVKDYFPEPVTVSWNSG | |
| | ALTSGVHTFPAVLQSSGLYSLSSVVTVPSSSLGTKTYTCNVD | |
| | HKPSNTKVDKRVESKYGPPCPPCPAPEFLGGPSVFLFPPKPK | |
| | DTLMISRTPEVTCVVVDVSQEDPEVQFNWYVDGVEVHNAK | |
| | TKPREEQFNSTYRVVSVLTVLHQDWLNGKEYKCKVSNKGL | |
| | PSSIEKTISKAKGQPREPQVYTLPPSQEEMTKNQVSLTCLVK | |
| | GFYPSDIAVEWESNGQPENNYKTTPPVLDSDGSFFLYSRLTV | |
| | DKSRWQEGNVFSCSVMHEALHNHYTQKSLSLSLGK | |
| Sintilimab, | DIQMTQSPSSVSASVGDRVTITCRASQGISSWLAWYQQKPG | 35 |
| Tyvyt, IBI-308 | KAPKLLISAASSLQSGVPSRFSGSGSGTDFTLTISSLQPEDFAT | |
| VL | YYCQQANHLPFTFGGGTKVEIKRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| Toripalimab, | QGQLVQSGAEVKKPGASVKVSCKASGYTFTDYEMHWVRQ | 36 |
| TeRuiPuLi, | APIHGLEWIGVIESETGGTAYNQKFKGRVTITADKSTSTAYM | |
| Terepril, Tuoyi, | ELSSLRSEDTAVYYCAREGITTVATTYYWYFDVWGQGTTV | |
| JS-001, TAB- | TVSSASTKGPSVFPLAPCSRSTSESTAALGCLVKDYFPEPVT | |
| 001 | VSWNSGALTSGVHTFPAVLQSSGLYSLSSVVTVPSSSLGTKT | |
| VH | YTCNVDHKPSNTKVDKRVESKYGPPCPPCPAPEFLGGPSVF | |
| | LFPPKPKDTLMISRTPEVTCVVVDVSQEDPEVQFNWYVDGV | |
| | EVHNAKTKPREEQFNSTYRVVSVLTVLHQDWLNGKEYKCK | |
| | VSNKGLPSSIEKTISKAKGQPREPQVYTLPPSQEEMTKNQVS | |
| | LTCLVKGFYPSDIAVEWESNGQPENNYKTTPPVLDSDGSFFL | |
| | YSRLTVDKSRWQEGNVFSCSVMHEALHNHYTQKSLSLSLG | |
| | K | |
| Toripalimab, | DVVMTQSPLSLPVTLGQPASISCRSSQSIVHSNGNTYLEWYL | 37 |
| TeRuiPuLi, | QKPGQSPQLLIYKVSNRFSGVPDRFSGSGSGTDFTLKISRVE | |
| Terepril, Tuoyi, | AEDVGVYYCFQGSHVPLTFGQGTKLEIKRTVAAPSVFIFPPS | |
| JS-001, TAB- | DEQLKSGTASVVCLLNNFYPREAKVQWKVDNALQSGNSQE | |
| 001 | SVTEQDSKDSTYSLSSTLTLSKADYEKHKVYACEVTHQGLS | |
| VL | SPVTKSFNRGEC | |
| Camrelizumab, | EVQLVESGGGLVQPGGSLRLSCAASGFTFSSYMMSWVRQA | 38 |
| HR-301210, | PGKGLEWVATISGGGANTYYPDSVKGRFTISRDNAKNSLYL | |
| INCSHR-01210, | QMNSLRAEDTAVYYCARQLYYFDYWGQGTTVTVSSASTK | |
| SHR-1210 | GPSVFPLAPCSRSTSESTAALGCLVKDYFPEPVTVSWNSGAL | |
| VH | TSGVHTFPAVLQSSGLYSLSSVVTVPSSSLGTKTYTCNVDHK | |
| | PSNTKVDKRVESKYGPPCPPCPAPEFLGGPSVFLFPPKPKDT | |
| | LMISRTPEVTCVVVDVSQEDPEVQFNWYVDGVEVHNAKTK | |
| | PREEQFNSTYRVVSVLTVLHQDWLNGKEYKCKVSNKGLPS | |
| | SIEKTISKAKGQPREPQVYTLPPSQEEMTKNQVSLTCLVKGF | |
| | YPSDIAVEWESNGQPENNYKTTPPVLDSDGSFFLYSRLTVD | |
| | KSRWQEGNVFSCSVMHEALHNHYTQKSLSLSLGK | |
| Camrelizumab, | DIQMTQSPSSLSASVGDRVTITCLASQTIGTWLTWYQQKPG | 39 |
| HR-301210, | KAPKLLIYTATSLADGVPSRFSGSGSGTDFTLTISSLQPEDFA | |
| INCSHR-01210, | TYYCQQVYSIPWTFGGGTKVEIKRTVAAPSVFIFPPSDEQLK | |
| SHR-1210 | SGTASVVCLLNNFYPREAKVQWKVDNALQSGNSQESVTEQ | |
| VL | DSKDSTYSLSSTLTLSKADYEKHKVYACEVTHQGLSSPVTK | |
| | SFNRGEC | |
| Cemiplimab, | EVQLLESGGVLVQPGGSLRLSCAASGFTFSNFGMTWVRQAP | 40 |
| Cemiplimab- | GKGLEWVSGISGGGRDTYFADSVKGRFTISRDNSKNTLYLQ | |
| rwlc, | MNSLKGEDTAVYYCVKWGNIYFDYWGQGTLVTVSSASTK | |
| LIBTAYO ®, | GPSVFPLAPCSRSTSESTAALGCLVKDYFPEPVTVSWNSGAL | |
| 6QVL057INT, | TSGVHTFPAVLQSSGLYSLSSVVTVPSSSLGTKTYTCNVDHK | |
| H4H7798N, | PSNTKVDKRVESKYGPPCPPCPAPEFLGGPSVFLFPPKPKDT | |
| | LMISRTPEVTCVVVDVSQEDPEVQFNWYVDGVEVHNAKTK | |
| | PREEQFNSTYRVVSVLTVLHQDWLNGKEYKCKVSNKGLPS | |
| REGN-2810, | SIEKTISKAKGQPREPQVYTLPPSQEEMTKNQVSLTCLVKGF | |
| SAR-439684 | YPSDIAVEWESNGQPENNYKTTPPVLDSDGSFFLYSRLTVD | |
| VH | KSRWQEGNVFSCSVMHEALHNHYTQKSLSLSLGK | |
| Cemiplimab, | DIQMTQSPSSLSASVGDSITITCRASLSINTFLNWYQQKPGKA | 41 |
| Cemiplimab- | PNLLIYAASSLHGGVPSRFSGSGSGTDFTLTIRTLQPEDFATY | |
| rwlc, | YCQQSSNTPFTFGPGTVVDFRRTVAAPSVFIFPPSDEQLKSGT | |
| LIBTAYO ®, | ASVVCLLNNFYPREAKVQWKVDNALQSGNSQESVTEQDSK | |
| 6QVL057INT, | DSTYSLSSTLTLSKADYEKHKVYACEVTHQGLSSPVTKSFN | |
| H4H7798N, | RGEC | |
| REGN-2810, | (Disulfide bridge) | |
| SAR-439684 | H22-H96, H131-L214, H144-H200, H223-H′223, | |
| VL | H226-H′226, H258-H318, H364-H422, H′22-H′96, | |
| | H′131-L′214, H′114-H′200, H′258-H′318, H′364- | |
| | H′422, L23-L88, L134-L194, L′23-L′88, L′134- | |
| | L′194) | |
| Lambrolizumab, | QVQLVQSGVEVKKPGASVKVSCKASGYTFTNYYMYWVRQ | 46 |
| Pembrolizumab, | APGQGLEWMGGINPSNGGTNFNEKFKNRVTLTTDSSTTTAY | |
| KEYTRUDA ®, | MELKSLQFDDTAVYYCARRDYRFDMGFDYWGQGTTVTVS | |
| MK-3475, SCH- | SASTKGPSVFPLAPCSRSTSESTAALGCLVKDYFPEPVTVSW | |
| 900475, | NSGALTSGVHTFPAVLQSSGLYSLSSVVTVPSSSLGTKTYTC | |
| h409A11 | NVDHKPSNTKVDKRVESKYGPPCPPCPAPEFLGGPSVFLFPP | |
| VH | KPKDTLMISRTPEVTCVVVDVSQEDPEVQFNWYVDGVEVH | |
| | NAKTKPREEQFNSTYRVVSVLTVLHQDWLNGKEYKCKVSN | |
| | KGLPSSIEKTISKAKGQPREPQVYTLPPSQEEMTKNQVSLTC | |
| | LVKGFYPSDIAVEWESNGQPENNYKTTPPVLDSDGSFFLYSR | |
| | LTVDKSRWQEGNVFSCSVMHEALHNHYTQKSLSLSLGK | |
| Lambrolizumab, | EIVLTQSPATLSLSPGERATLSCRASKGVSTSGYSYLHWYQQ | 47 |
| Pembrolizumab, | KPGQAPRLLIYLASYLESGVPARFSGSGSGTDFTLTISSLEPE | |
| KEYTRUDA ®, | DFAVYYCQHSRDLPLTFGGGTKVEIKRTVAAPSVFIFPPSDE | |
| MK-3475, SCH- | QLKSGTASVVCLLNNFYPREAKVQWKVDNALQSGNSQESV | |
| 900475, | TEQDSKDSTYSLSSTLTLSKADYEKHKVYACEVTHQGLSSP | |
| h409A11 | VTKSFNRGEC | |
| VL | | |
| Lambrolizumab, | QVQLVQSGVEVKKPGASVKVSCKASGYTFTNYYMYWVRQ | 48 |
| Pembrolizumab, | APGQGLEWMGGFPSNGGTNFNEKFKNRVTLTTDSSTTTAY | |
| KEYTRUDA ®, | MELKSLQFDDTAVYYCARRDYRFDMGFDYWGQGTTVTVS | |
| MK-3475, SCH- | S | |
| 900475, | (Disulfide bridge) | |
| h409A11 | H22-H96, H134-L218, H147-H203, H226-H′226, | |
| VH | H229-H′229, H261-H321, H367-H425, H′22-H′96, | |
| | H′134-L′218, H′147-H′203, H′261-H′321, H′367- | |
| | H′425, L23-L92, L138-L198, L′23-L′92, L′138- | |
| | L′198) | |
| Lambrolizumab, | EIVLTQSPATLSLSPGERATLSCRASKGVSTSGYSYLHWYQQ | 49 |
| Pembrolizumab, | KPGQAPRLLIYLASYLESGVPARFSGSGSGTDFTLTISSLEPE | |
| KEYTRUDA ®, | DFAVYYCQHSRDLPLTFGGGTKVEIK | |
| MK-3475, SCH- | | |
| 900475, | | |
| h409A11 | | |
| VL | | |
| Nivolumab, | QVQLVESGGGVVQPGRSLRLDCKASGITFSNSGMHWVRQA | 50 |
| Nivolumab | PGKGLEWVAVIWYDGSKRYYADSVKGRFTISRDNSKNTLF | |
| BMS, | LQMNSLRAEDTAVYYCATNDDYWGQGTLVTVSS | |
| OPDIVO ®, | | |
| BMS-936558, | | |
| MDX-1106, | | |
| ONO-4538 | | |
| VH | | |
| Nivolumab, | EIVLTQSPATLSLSPGERATLSCRASQSVSSYLAWYQQKPGQ | 51 |
| Nivolumab | APRLLIYDASNRATGIPARFSGSGSGTDFTLTISSLEPEDFAV | |
| BMS, | YYCQQSSNWPRTFGQGTKVEIK | |
| OPDIVO ®, | (Disulfide bridge) | |
| BMS-936558, | H22-H96, H127-L214, H140-H196, H219-H′219, | |
| MDX-1106, | H222-H′222, H254-H314, H360-H418, H′22-H′96, | |
| ONO-4538 | H′127-L′214, H′140-H′196, H′254-H′314, H′360- | |
| VL | H′418, L-23-L88, L134-L194, L′23-L′88, L′134- | |
| | L′194) | |
| Prolgolimab, | QVQLVQSGGGLVQPGGSLRLSCAASGFTFSSYWMYWVRQ | 52 |
| Forteca, BCD- | VPGKGLEWVSAIDTGGGRTYYADSVKGRFAISRVNAKNTM | |
| 100 | YLQMNSLRAEDTAVYYCARDEGGGTGWGVLKDWPYGLD | |
| VH | AWGQGTLVTVSSASTKGPSVFPLAPSSKSTSGGTAALGCLV | |
| | KDYFPEPVTVSWNSGALTSGVHTFPAVLQSSGLYSLSSVVT | |
| | VPSSSLGTQTYICNVNHKPSNTKVDKRVEPKSCDKTHTCPPC | |
| | PAPEAAGGPSVFLFPPKPKDTLMISRTPEVTCVVVDVSHEDP | |
| | EVKFNWYVDGVEVHNAKTKPREEQYNSTYRVVSVLTVLH | |
| | QDWLNGKEYKCKVSNKALPAPIEKTISKAKGQPREPQVYTL | |
| | PPSREEMTKNQVSLTCLVKGFYPSDIAVEWESNGQPENNYK | |
| | TTPPVLDSDGSFFLYSKLTVDKSRWQQGNVFSCSVMHEALH | |
| | NHYTQKSLSLSPGK | |
| Prolgolimab, | QPVLTQPLSVSVALGQTARITCGGNNIGSKNVHWYQQKPG | 53 |
| Forteca, BCD- | QAPVLVIYRDSNRPSGIPERFSGSNSGNTATLTISRAQAGDEA | |
| 100 | DYYCQVWDSSTAVFGTGTKLTVLQRTVAAPSVFIFPPSDEQ | |
| VL | LKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| Balstilimab, | QVQLVESGGGVVQPGRSLRLSCAASGFTFSSYGMHWVRQA | 54 |
| 1Q2QT5M7EO, | PGKGLEWVAVIWYDGSNKYYADSVKGRFTISRDNSKNTLY | |
| AGEN-2034, | LQMNSLRAEDTAVYYCASNGDHWGQGTLVTVSSASTKGPS | |
| AGEN-2034w | VFPLAPCSRSTSESTAALGCLVKDYFPEPVTVSWNSGALTSG | |
| VH | VHTFPAVLQSSGLYSLSSVVTVPSSSLGTKTYTCNVDHKPSN | |
| | TKVDKRVESKYGPPCPPCPAPEFLGGPSVFLFPPKPKDTLMIS | |
| | RTPEVTCVVVDVSQEDPEVQFNWYVDGVEVHNAKTKPREE | |
| | QFNSTYRVVSVLTVLHQDWLNGKEYKCKVSNKGLPSSIEKT | |
| | ISKAKGQPREPQVYTLPPSQEEMTKNQVSLTCLVKGFYPSDI | |
| | AVEWESNGQPENNYKTTPPVLDSDGSFFLYSRLTVDKSRW | |
| | QEGNVFSCSVMHEALHNHYTQKSLSLSLG | |
| Balstilimab, | EIVMTQSPATLSVSPGERATLSCRASQSVSSNLAWYQQKPG | 55 |
| 1Q2QT5M7EO, | QAPRLLIYGASTRATGIPARFSGSGSGTEFTLTISSLQSEDFAV | |
| AGEN-2034, | YYCQQYNNWPRTFGQGTKVEIKRTVAAPSVFIFPPSDEQLK | |
| AGEN-2034w | SGTASVVCLLNNFYPREAKVQWKVDNALQSGNSQESVTEQ | |
| VL | DSKDSTYSLSSTLTLSKADYEKHKVYACEVTHQGLSSPVTK | |
| | SFNRGEC | |
| Dostarlimab, | EVQLLESGGGLVQPGGSLRLSCAASGFTFSSYDMSWVRQAP | 56 |
| ANB-011, | GKGLEWVSTISGGGSYTYYQDSVKGRFTISRDNSKNTLYLQ | |
| | MNSLRAEDTAVYYCASPYYAMDYWGQGTTVTVSSASTKG | |
| | PSVFPLAPCSRSTSESTAALGCLVKDYFPEPVTVSWNSGALT | |
| | SGVHTFPAVLQSSGLYSLSSVVTVPSSSLGTKTYTCNVDHKP | |
| | SNTKVDKRVESKYGPPCPPCPAPEFLGGPSVFLFPPKPKDTL | |
| GSK- | MISRTPEVTCVVVDVSQEDPEVQFNWYVDGVEVHNAKTKP | |
| 4057190A, | REEQFNSTYRVVSVLTVLHQDWLNGKEYKCKVSNKGLPSSI | |
| P0GVQ9A4S5, | EKTISKAKGQPREPQVYTLPPSQEEMTKNQVSLTCLVKGFY | |
| TSR-042, WBP- | PSDIAVEWESNGQPENNYKTTPPVLDSDGSFFLYSRLTVDKS | |
| 285 | RWQEGNVFSCSVMHEALHNHYTQKSLSLSLGK | |
| VH | | |
| Dostarlimab, | DIQLTQSPSFLSAYVGDRVTITCKASQDVGTAVAWYQQKPG | 57 |
| ANB-011, | KAPKLLIYWASTLHTGVPSRFSGSGSGTEFTLTISSLQPEDFA | |
| GSK- | TYYCQHYSSYPWTFGQGTKLEIKRTVAAPSVFIFPPSDEQLK | |
| 4057190A, | SGTASVVCLLNNFYPREAKVQWKVDNALQSGNSQESVTEQ | |
| P0GVQ9A4S5, | DSKDSTYSLSSTLTLSKADYEKHKVYACEVTHQGLSSPVTK | |
| TSR-042, WBP- | SFNRGEC | |
| 285 | | |
| VL | | |
| Serplulimab, | QVQLVESGGGLVKPGGSLRLSCAASGFTFSNYGMSWIRQAP | 58 |
| HLX-10 | GKGLEWVSTISGGGSNIYYADSVKGRFTISRDNAKNSLYLQ | |
| VH | MNSLRAEDTAVYYCVSYYYGIDFWGQGTSVTVSSASTKGP | |
| | SVFPLAPCSRSTSESTAALGCLVKDYFPEPVTVSWNSGALTS | |
| | GVHTFPAVLQSSGLYSLSSVVTVPSSSLGTKTYTCNVDHKPS | |
| | NTKVDKRVESKYGPPCPPCPAPEFLGGPSVFLFPPKPKDTLM | |
| | ISRTPEVTCVVVDVSQEDPEVQFNWYVDGVEVHNAKTKPR | |
| | EEQFNSTYRVVSVLTVLHQDWLNGKEYKCKVSKGLPSSIEK | |
| | TISKAKGQPREPQVYTLPPSQEEMTKNQVSLTCLVKGFYPSD | |
| | IAVEWESNGQPENNYKTTPPVLDSDGSFFLYSRLTVDKSRW | |
| | QEGNVFSCSVMHEALHNHYTQKSLSLSLGK | |
| Serplulimab, | DIQMTQSPSSLSASVGDRVTITCKASQDVTTAVAWYQQKPG | 59 |
| HLX-10 | KAPKLLIYWASTRHTGVPSRFSGSGSGTDFTLTISSLQPEDFA | |
| VL | TYYCQQHYTIPWTFGGGTKLEIKRTVAAPSVFIFPPSDEQLK | |
| | SGTASVVCLLNNFYPREAKVQWKVDNALQSGNSQESVTEQ | |
| | DSKDSTYSLSSTLTLSKADYEKHKVYACEVTHQGLSSPVTK | |
| | SFNRGEC | |
| Retifanlimab, | QVQLVQSGAEVKKPGASVKVSCKASGYSFTSYWMNWVRQ | 60 |
| 2Y3T5IF01Z, | APGQGLEWIGVIHPSDSETWLDQKFKDRVTITVDKSTSTAY | |
| INCMGA- | MELSSLRSEDTAVYYCAREHYGTSPFAYWGQGTLVTVSSAS | |
| 00012, | TKGPSVFPLAPCSRSTSESTAALGCLVKDYFPEPVTVSWNSG | |
| INCMGA-0012, | ALTSGVHTFPAVLQSSGLYSLSSVVTVPSSSLGTKTYTCNVD | |
| MGA-012 | HKPSNTKVDKRVESKYGPPCPPCPAPEFLGGPSVFLFPPKPK | |
| VH | DTLMISRTPEVTCVVVDVSQEDPEVQFNWYVDGVEVHNAK | |
| | TKPREEQFNSTYRVVSVLTVLHQDWLNGKEYKCKVSNKGL | |
| | PSSIEKTISKAKGQPREPQVYTLPPSQEEMTKNQVSLTCLVK | |
| | GFYPSDIAVEWESNGQPENNYKTTPPVLDSDGSFFLYSRLTV | |
| | DKSRWQEGNVFSCSVMHEALHNHYTQKSLSLSLG | |
| Retifanlimab, | EIVLTQSPATLSLSPGERATLSCRASESVDNYGMSFMNWFQ | 61 |
| 2Y3T5IF01Z, | QKPGQPPKLLIHAASNQGSGVPSRFSGSGSGTDFTLTISSLEP | |
| INCMGA- | EDFAVYFCQQSKEVPYTFGGGTKVEIKRTVAAPSVFIFPPSD | |
| 00012, | EQLKSGTASVVCLLNNFYPREAKVQWKVDNALQSGNSQES | |
| INCMGA-0012, | VTEQDSKDSTYSLSSTLTLSKADYEKHKVYACEVTHQGLSS | |
| MGA-012 | PVTKSFNRGEC | |
| VL | | |
| Sasanlimab, | QVQLVQSGAEVKKPGASVKVSCKASGYTFTSYWINWVRQA | 62 |
| LZZ0IC2EWP, | PGQGLEWMGNIYPGSSLTNYNEKFKNRVTMTRDTSTSTVY | |
| PF-06801591, | MELSSLRSEDTAVYYCARLSTGTFAYWGQGTLVTVSSASTK | |
| RN-888 | GPSVFPLAPCSRSTSESTAALGCLVKDYFPEPVTVSWNSGAL | |
| VH | TSGVHTFPAVLQSSGLYSLSSVVTVPSSSLGTKTYTCNVDHK | |
| | PSNTKVDKRVESKYGPPCPPCPAPEFLGGPSVFLFPPKPKDT | |
| | LMISRTPEVTCVVVDVSQEDPEVQFNWYVDGVEVHNAKTK | |
| | PREEQFNSTYRVVSVLTVLHQDWLNGKEYKCKVSNKGLPS | |
| | SIEKTISKAKGQPREPQVYTLPPSQEEMTKNQVSLTCLVKGF | |
| | YPSDIAVEWESNGQPENNYKTTPPVLDSDGSFFLYSRLTVD | |
| | KSRWQEGNVFSCSVMHEALHNHYTQKSLSLSLGK | |
| Sasanlimab, | DIVMTQSPDSLAVSLGERATINCKSSQSLWDSGNQKNFLTW | 63 |
| LZZ0IC2EWP, | YQQKPGQPPKLLIYWTSYRESGVPDRFSGSGSGTDFTLTISSL | |
| PF-06801591, | QAEDVAVYYCQNDYFYPHTFGGGTKVEIKRTVAAPSVFIFP | |
| RN-888 | PSDEQLKSGTASVVCLLNNFYPREAKVQWKVDNALQSGNS | |
| VL | QESVTEQDSKDSTYSLSSTLTLSKADYEKHKVYACEVTHQG | |
| | LSSPVTKSFNRGEC | |
| Spartalizumab, | EVQLVQSGAEVKKPGESLRISCKGSGYTFTTYWMHWVRQA | 64 |
| NVP-LZV-184, | TGQGLEWMGNIYPGTGGSNFDEKFKNRVTITADKSTSTAY | |
| PDR-001, | MELSSLRSEDTAVYYCTRWTTGTGAYWGQGTTVTVSSAST | |
| QOG25L6Z8Z | KGPSVFPLAPCSRSTSESTAALGCLVKDYFPEPVTVSWNSGA | |
| VH | LTSGVHTFPAVLQSSGLYSLSSVVTVPSSSLGTKTYTCNVDH | |
| | KPSNTKVDKRVESKYGPPCPPCPAPEFLGGPSVFLFPPKPKD | |
| | TLMISRTPEVTCVVVDVSQEDPEVQFNWYVDGVEVHNAKT | |
| | KPREEQFNSTYRVVSVLTVLHQDWLNGKEYKCKVSNKGLP | |
| | SSIEKTISKAKGQPREPQVYTLPPSQEEMTKNQVSLTCLVKG | |
| | FYPSDIAVEWESNGQPENNYKTTPPVLDSDGSFFLYSRLTVD | |
| | KSRWQEGNVFSCSVMHEALHNHYTQKSLSLSLG | |
| Spartalizumab, | EIVLTQSPATLSLSPGERATLSCKSSQSLLDSGNQKNFLTWY | 65 |
| NVP-LZV-184, | QQKPGQAPRLLIYWASTRESGVPSRFSGSGSGTDFTFTISSLE | |
| PDR-001, | AEDAATYYCQNDYSYPYTFGQGTKVEIKRTVAAPSVFIFPPS | |
| QOG25L6Z8Z | DEQLKSGTASVVCLLNNFYPREAKVQWKVDNALQSGNSQE | |
| VL | SVTEQDSKDSTYSLSSTLTLSKADYEKHKVYACEVTHQGLS | |
| | SPVTKSFNRGEC | |
| Cetrelimab, | QVQLVQSGAEVKKPGSSVKVSCKASGGTFSSYAISWVRQAP | 66 |
| JNJ-3283, JNJ- | GQGLEWMGGIIPIFDTANYAQKFQGRVTITADESTSTAYME | |
| 63723283, | LSSLRSEDTAVYYCARPGLAAAYDTGSLDYWGQGTLVTVS | |
| LYK98WP91F | SASTKGPSVFPLAPCSRSTSESTAALGCLVKDYFPEPVTVSW | |
| VH | NSGALTSGVHTFPAVLQSSGLYSLSSVVTVPSSSLGTKTYTC | |
| | NVDHKPSNTKVDKRVESKYGPPCPPCPAPEFLGGPSVFLFPP | |
| | KPKDTLMISRTPEVTCVVVDVSQEDPEVQFNWYVDGVEVH | |
| | NAKTKPREEQFNSTYRVVSVLTVLHQDWLNGKEYKCKVSN | |
| | KGLPSSIEKTISKAKGQPREPQVYTLPPSQEEMTKNQVSLTC | |
| | LVKGFYPSDIAVEWESNGQPENNYKTTPPVLDSDGSFFLYSR | |
| | LTVDKSRWQEGNVFSCSVMHEALHNHYTQKSLSLSLGK | |
| Cetrelimab, | EIVLTQSPATLSLSPGERATLSCRASQSVRSYLAWYQQKPGQ | 67 |
| JNJ-3283, JNJ- | APRLLIYDASNRATGIPARFSGSGSGTDFTLTISSLEPEDFAV | |
| 63723283, | YYCQQRNYWPLTFGQGTKVEIKRTVAAPSVFIFPPSDEQLKS | |
| LYK98WP91F | GTASVVCLLNNFYPREAKVQWKVDNALQSGNSQESVTEQD | |
| VL | SKDSTYSLSSTLTLSKADYEKHKVYACEVTHQGLSSPVTKSF | |
| | NRGEC | |
| Tebotelimab, | DIQMTQSPSSLSASVGDRVTITCRASQDVSSWAWYQQKPGK | 68 |
| MGD-013 | APKLLIYSASYRYTGVPSRFSGSGSGTDFTLTISSLQPEDFAT | |
| VH | YYCQQHYSTPWTFGGGTKLEIKGGGSGGGGQVQLVQSGAE | |
| | VKKPGASVKVSCKASGYSFTSYWMNWVRQAPGQGLEWIG | |
| | VIHPSDSETWLDQKFKDRVTITVDKSTSTAYMELSSLRSEDT | |
| | AVYYCAREHYGTSPFAYWGQGTLVTVSSGGCGGGEVAAC | |
| | EKEVAALEKEVAALEKEVAALEKESKYGPPCPPCPAPEFLG | |
| | GPSVFLFPPKPKDTLYITREPEVTCWVDVSQEDPEVQFNWY | |
| | VDGVEVHNAKTKPREEQFNSTYRWSVLTVLHQDWLNGKE | |
| | YKCKVSNKGLPSSIEKTISKAKGQPREPQVYTLPPSQEEMTK | |
| | NQVSLTCLVKGFYPSDIAVEWESNGQPENNYKTTPPVLDSD | |
| | GSFFLYSRLTVDKSRWQEGNVFSCSVMHEALHNHYTQKSL | |
| | SLSLG | |
| Tebotelimab, | EIVLTQSPATLSLSPGERATLSCRASESVDNYGMSEMNWFQ | 69 |
| MGD-013 | QKPGQPPKLLIHAASNQGSGVPSRFSGSGSGTDFTLTISSLEP | |
| VL | EDFAVYFCQQSKEVPYTFGGGTKVEIKGGGSGGGGQVQLV | |
| | QSGAEVKKPGASVKVSCKASGYTFTDYNMDWVRQAPGQG | |
| | LEWMGDINPDNGVTIYNQKFEGRVTMTTDTSTSTAYMELR | |
| | SLRSDDTAVYYCAREADYFYFDYWGQGTTLTVSSGGCGGG | |
| | KVAACKEKVAALKEKVAALKEKVAALKE | |
| Pidilizumab, | QVQLVQSGSELKKPGASVKISCKASGYTFTNYGMNWVRQA | 70 |
| CT-011, MDV- | PGQGLQWMGWINTDSGESTYAEEFKGRFVFSLDTSVNTAY | |
| 9300 | LQITSLTAEDTGMYFCVRVGYDALDYWGQGTLVTVSSAST | |
| VH | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPELLGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| Pidilizumab, | EIVLTQSPSSLSASVGDRVTITCSARSSVSYMHWFQQKPGKA | 71 |
| CT-011, MDV- | PKLWIYRTSNLASGVPSRFSGSGSGTSYCLTINSLQPEDFATY | |
| 9300 | YCQQRSSFPLTFGGGTKLEIKRTVAAPSVFIFPPSDEQLKSGT | |
| VL | ASVVCLLNNFYPREAKVQWKVDNALQSGNSQESVTEQDSK | |
| | DSTYSLSSTLTLSKADYEKHKVYACEVTHQGLSSPVTKSFN | |
| | RGEC | |
| SG-001 VH | QVQLVESGGGVVQPGRSLRLTCKASGLTFSSSGMHWVRQA | 72 |
| | PGKGLEWVAVIWYDGSKRYYADSVKGRFTISRDNSKNTLF | |
| | LQMNSLRAEDTAVYYCATNNDYWGQGTLVTVSS | |
| SG-001 VL | EIVLTQSPATLSLSPGERATLSCRASQSVSSYLAWYQQKPGQ | 73 |
| | APRLLIYTASNRATGIPARFSGSGSGTDFTLTISSLEPEDFAVY | |
| | YCQQYSNWPRTFGQGTKVEIK | |
| mpLZM-009 | EVQLQQSGPVLVKPGASVKMSCKASGYTFTSYYMYWVKQ | 74 |
| VH | SHGKSLEWIGGVNPSNGGTNFNEKFKSKATLTVDKSSSTAY | |
| (Murine | MELNSLTSEDSAVYYCARRDYRYDMGFDYWGQGTTLTVSS | |
| Precursor of | | |
| LZM-009) | | |
| mpLZM-009 | QIVLTQSPAIMSASPGEKVTMTCRASKGVSTSGYSYLHWYQ | 75 |
| VL | QKPGSSPRLLIYLASYLESGVPVRFSGSGSGTSYSLTISRMEA | |
| (Murine | EDAATYYCQHSRELPLTFGTGTRLEIK | |
| Precursor of | | |
| LZM-009) | | |
| LZM-009 VH | QVQLVQSGAEVKKPGASVKVSCKASGYTFTSYYMYWVRQ | 76 |
| | APGQGLEWMGGVNPSNGGTNFNEKFKSRVTITADKSTSTA | |
| | YMELSSLRSEDTAVYYCARRDYRYDMGFDYWGQGTTVTV | |
| | SS | |
| LZM-009 VL | EIVLTQSPATLSLSPGERATISCRASKGVSTSGYSYLHWYQQ | 77 |
| | KPGQAPRLLIYLASYLESGVPARFSGSGSGTDFTLTISSLEPE | |
| | DFATYYCQHSRELPLTFGTGTKVEIK | |
| Budigalimab, | EIQLVQSGAEVKKPGSSVKVSCKASGYTFTHYGMNWVRQA | 78 |
| 6VDO4TY3OO, | PGQGLEWVGWVNTYTGEPTYADDFKGRLTFTLDTSTSTAY | |
| ABBV-181, PR- | MELSSLRSEDTAVYYCTREGEGLGFGDWGQGTTVTVSSAST | |
| 1648817 | KGPSVFPLAPSSKSTSGGTAALGCLVKDYFPEPVTVSWNSG | |
| VH | ALTSGVHTFPAVLQSSGLYSLSSVVTVPSSSLGTQTYICNVN | |
| | HKPSNTKVDKKVEPKSCDKTHTCPPCPAPEAAGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| Budigalimab, | DVVMTQSPLSLPVTPGEPASISCRSSQSIVHSHGDTYLEWYL | 79 |
| 6VDO4TY3OO, | QKPGQSPQLLIYKVSNRFSGVPDRFSGSGSGTDFTLKISRVE | |
| ABBV-181, PR- | AEDVGVYYCFQGSHIPVTFGQGTKLEIKRTVAAPSVFIFPPS | |
| 1648817 | DEQLKSGTASVVCLLNNFYPREAKVQWKVDNALQSGNSQE | |
| VL | SVTEQDSKDSTYSLSSTLTLSKADYEKHKVYACEVTHQGLS | |
| | SPVTKSFNRGEC | |
| Lambrolizumab, | NYYMY | 80 |
| Pembrolizumab, | | |
| KEYTRUDA ®, | | |
| MK-3475, SCH- | | |
| 900475, | | |
| h409A11 | | |
| VH CDR1 | | |
| Lambrolizumab, | GINPSNGGTNFNEKFKN | 81 |
| Pembrolizumab, | | |
| KEYTRUDA ®, | | |
| MK-3475, SCH- | | |
| 900475, | | |
| h409A11 | | |
| VH CDR2 | | |
| Lambrolizumab, | RDYRFDMGFDY | 82 |
| Pembrolizumab, | | |
| KEYTRUDA ®, | | |
| MK-3475, SCH- | | |
| 900475, | | |
| h409A11 | | |
| VH CDR3 | | |
| Lambrolizumab, | RASKGVSTSGYSYLH | 83 |
| Pembrolizumab, | | |
| KEYTRUDA ®, | | |
| MK-3475, SCH- | | |
| 900475, | | |
| h409A11 | | |
| VL CDR1 | | |
| Lambrolizumab, | LASYLES | 84 |
| Pembrolizumab, | | |
| KEYTRUDA ®, | | |
| MK-3475, SCH- | | |
| 900475, | | |
| h409A11 | | |
| VL CDR2 | | |
| Lambrolizumab, | QHSRDLPLT | 85 |
| Pembrolizumab, | | |
| KEYTRUDA ®, | | |
| MK-3475, SCH- | | |
| 900475, | | |
| h409A11 | | |
| VL CDR3 | | |
| Nivolumab, | NSGMH | 86 |
| Nivolumab | | |
| BMS, | | |
| OPDIVO ®, | | |
| BMS-936558, | | |
| MDX-1106, | | |
| ONO-4538 | | |
| VH CDR1 | | |
| Nivolumab, | VIWYDGSKRYYADSVKG | 87 |
| Nivolumab | | |
| BMS, | | |
| OPDIVO ®, | | |
| BMS-936558, | | |
| MDX-1106, | | |
| ONO-4538 | | |
| VH CDR2 | | |
| Nivolumab, | NDDY | 88 |
| Nivolumab | | |
| BMS, | | |
| OPDIVO ®, | | |
| BMS-936558, | | |
| MDX-1106, | | |
| ONO-4538 | | |
| VH CDR3 | | |
| Nivolumab, | RASQSVSSYLA | 89 |
| Nivolumab | | |
| BMS, | | |
| OPDIVO ®, | | |
| BMS-936558, | | |
| MDX-1106, | | |
| ONO-4538 | | |
| VL CDR1 | | |
| Nivolumab, | DASNRAT | 90 |
| Nivolumab | | |
| BMS, | | |
| OPDIVO ®, | | |
| BMS-936558, | | |
| MDX-1106, | | |
| ONO-4538 | | |
| VL CDR2 | | |
| Nivolumab, | QQSSNWPRT | 91 |
| Nivolumab | | |
| BMS, | | |
| OPDIVO ®, | | |
| BMS-936558, | | |
| MDX-1106, | | |
| ONO-4538 | | |
| VL CDR3 | | |
| Serplulimab, | FTFSNYGMS | 92 |
| HLX-10 | | |
| VH CDR1 | | |
| Serplulimab, | TISGGGSNIY | 93 |
| HLX-10 | | |
| VH CDR2 | | |
| Serplulimab, | VSYYYGIDF | 94 |
| HLX-10 | | |
| VH CDR3 | | |
| Serplulimab, | KASQDVTTAVA | 95 |
| HLX-10 | | |
| VL CDR1 | | |
| Serplulimab, | WASTRHT | 96 |
| HLX-10 | | |
| VL CDR2 | | |
| Serplulimab, | QQHYTIPWT | 97 |
| HLX-10 | | |
| VL CDR3 | | |
| SG-001 | GLTFSSSG | 98 |
| VH CDR1 | | |
| SG-001 | IWYDGSKR | 99 |
| VH CDR2 | | |
| SG-001 | ATNNDY | 100 |
| VH CDR3 | | |
| SG-001 | RASQSVSSYLA | 101 |
| VL CDR1 | | |
| SG-001 | TASNRAT | 102 |
| VL CDR2 | | |
| SG-001 | QQYSNWPRT | 103 |
| VL CDR3 | | |
| PD-1-Fc- | MQIPQAPWPWWAVLQLGWRPGWFLDSPDRPWNPPTFSPAL | 104 |
| OX40L (Code), | LVVTEGDNATFTCSFSNTSESFVLNWYRMSPSNQTDKLAAF | |
| SL-279252 | PEDRSQPGQDCRFRVTQLPNGRDFHMSVVRARRNDSGTYL | |
| (Code), TAK- | CGAISLAPKAQIKESLRAELRVTERRAEVPTAHPSPSPRPAG | |
| 252 (Code) | QFQSKYGPPCPSCPAPEFLGGPSVFLFPPKPKDTLMISRTPEV | |
| | TCWVDVSQEDPEVQFNWYVDGVEVHNAKTKPREEQFNST | |
| | YRVVSVLTVLHQDWLSGKEYKCKVSSKGLPSSIEKTISNAT | |
| | GQPREPQVYTLPPSQEEMTKNQVSLTCLVKGFYPSDIAVEW | |
| | ESNGQPENNYKTTPPVLDSDGSFFLYSRLTVDKSSWQEGNV | |
| | FSCSVMHEALHNHYTQKSLSLSLGKIEGRMDQVSHRYPRIQ | |
| | SIKVQFTEYKKEKGFILTSQKEDEIMKVQNNSVIINCDGFYLI | |
| | SLKGYFSQEVNISLHYQKDEEPLFQLKKVRSVNSLMVASLT | |
| | YKDKVYLNVTTDNTSLDDFHVNGGELILIHQNPGEFCVL | |
| | MQIPQAPWPWWAVLQLGWRPGWFLDSPDRPWNPPTFSPAL | |
| | LVVTEGDNATFTCSFSNTSESFVLNWYRMSPSNQTDKLAAF | |
| | PEDRSQPGQDCRFRVTQLPNGRDFHMSVVRARRNDSGTYL | |
| | CGAISLAPKAQIKESLRAELRVTERRAEVPTAHPSPSPRPAG | |
| | QFQ | |
| | QVSHRYPRIQSIKVQFTEYKKEKGFILTSQKEDEIMKVQNNS | |
| | VIINCDGFYLISLKGYFSQEVNISLHYQKDEEPLFQLKKVRSV | |
| | NSLMVASLTYKDKVYLNVTTDNTSLDDFHVNGGELILIHQN | |
| | PGEFCVL | |

An anti-PD-1 polypeptide or an anti-PD-1 antigen binding fragment can comprise a VH having an amino acid sequence of any one of SEQ ID NOS: 32, 34, 36, 38, 40, 42, 44, 46, 48, 50, 52, 54, 56, 58, 60, 62, 64, 66, 68, 70, 72, 74, 76, and 78. An anti-PD-1 polypeptide or an anti-PD-1 antigen binding fragment can comprise a VH having an amino acid sequence of any one of SEQ ID NOS: 33, 35, 37, 39, 41, 43, 45, 47, 49, 51, 53, 55, 57, 59, 61, 63, 65, 67, 69, 71, 73, 75, 77, and 79.

In one instance, an anti-PD-1 polypeptide or an anti-PD-1 antigen binding fragment comprises a VH having an amino acid sequence of SEQ ID NO: 32, and a VL having an amino acid sequence of SEQ ID NO: 33. In another instance, an anti-PD-1 polypeptide or an anti-PD-1 antigen binding fragment comprises a VH having an amino acid sequence of SEQ ID NO: 34, and a VL having an amino acid sequence of SEQ ID NO: 35. In another instance, an anti-PD-1 polypeptide or an anti-PD-1 antigen binding fragment comprises a VH having an amino acid sequence of SEQ ID NO: 36, and a VL having an amino acid sequence of SEQ ID NO: 37. In another instance, an anti-PD-1 polypeptide or an anti-PD-1 antigen binding fragment comprises a VH having an amino acid sequence of SEQ ID NO: 38, and a VL having an amino acid sequence of SEQ ID NO: 39. In another instance, an anti-PD-1 polypeptide or an anti-PD-1 antigen binding fragment comprises a VH having an amino acid sequence of SEQ ID NO: 40, and a VL having an amino acid sequence of SEQ ID NO: 41. In another instance, an anti-PD-1 polypeptide or an anti-PD-1 antigen binding fragment comprises a VH having an amino acid sequence of SEQ ID NO: 42, and a VL having an amino acid sequence of SEQ ID NO: 43. In another instance, an anti-PD-1 polypeptide or an anti-PD-1 antigen binding fragment comprises a VH having an amino acid sequence of SEQ ID NO: 44, and a VL having an amino acid sequence of SEQ ID NO: 45. In another instance, an anti-PD-1 polypeptide or an anti-PD-1 antigen binding fragment comprises a VH having an amino acid sequence of SEQ ID NO: 46, and a VL having an amino acid sequence of SEQ ID NO: 47. In another instance, an anti-PD-1 polypeptide or an anti-PD-1 antigen binding fragment comprises a VH having an amino acid sequence of SEQ ID NO: 48, and a VL having an amino acid sequence of SEQ ID NO: 49. In another instance, an anti-PD-1 polypeptide or an anti-PD-1 antigen binding fragment comprises a VH having an amino acid sequence of SEQ ID NO: 50, and a VL having an amino acid sequence of SEQ ID NO: 51. In another instance, an anti-PD-1 polypeptide or an anti-PD-1 antigen binding fragment comprises a VH having an amino acid sequence of SEQ ID NO: 52, and a VL having an amino acid sequence of SEQ ID NO: 53. In another instance, an anti-PD-1 polypeptide or an anti-PD-1 antigen binding fragment comprises a VH having an amino acid sequence of SEQ ID NO: 54, and a VL having an amino acid sequence of SEQ ID NO: 55. In another instance, an anti-PD-1 polypeptide or an anti-PD-1 antigen binding fragment comprises a VH having an amino acid sequence of SEQ ID NO: 56, and a VL having an amino acid sequence of SEQ ID NO: 57. In another instance, an anti-PD-1 polypeptide or an anti-PD-1 antigen binding fragment comprises a VH having an amino acid sequence of SEQ ID NO: 58, and a VL having an amino acid sequence of SEQ ID NO: 59. In another instance, an anti-PD-1 polypeptide or an anti-PD-1 antigen binding fragment comprises a VH having an amino acid sequence of SEQ ID NO: 60, and a VL having an amino acid sequence of SEQ ID NO: 61. In another instance, an anti-PD-1 polypeptide or an anti-PD-1 antigen binding fragment comprises a VH having an amino acid sequence of SEQ ID NO: 62, and a VL having an amino acid sequence of SEQ ID NO: 63. In another instance, an anti-PD-1 polypeptide or an anti-PD-1 antigen binding fragment comprises a VH having an amino acid sequence of SEQ ID NO: 64, and a VL having an amino acid sequence of SEQ ID NO: 65. In another instance, an anti-PD-1 polypeptide or an anti-PD-1 antigen binding fragment comprises a VH having an amino acid sequence of SEQ ID NO: 66, and a VL having an amino acid sequence of SEQ ID NO: 67. In another instance, an anti-PD-1 polypeptide or an anti-PD-1 antigen binding fragment comprises a VH having an amino acid sequence of SEQ ID NO: 68, and a VL having an amino acid sequence of SEQ ID NO: 69. In another instance, an anti-PD-1 polypeptide or an anti-PD-1 antigen binding fragment comprises a VH having an amino acid sequence of SEQ ID NO: 70, and a VL having an amino acid sequence of SEQ ID NO: 71. In another instance, an anti-PD-1 polypeptide or an anti-PD-1 antigen binding fragment comprises a VH having an amino acid sequence of SEQ ID NO: 72, and a VL having an amino acid sequence of SEQ ID NO: 73. In another instance, an anti-PD-1 polypeptide or an anti-PD-1 antigen binding fragment comprises a VH having an amino acid sequence of SEQ ID NO: 74, and a VL having an amino acid sequence of SEQ ID NO: 75. In another instance, an anti-PD-1 polypeptide or an anti-PD-1 antigen binding fragment comprises a VH having an amino acid sequence of SEQ ID NO: 76, and a VL having an amino acid sequence of SEQ ID NO: 77. In another instance, an anti-PD-1 polypeptide or an anti-PD-1 antigen binding fragment comprises a VH having an amino acid sequence of SEQ ID NO: 78, and a VL having an amino acid sequence of SEQ ID NO: 79.

In one instance, an anti-PD-1 polypeptide or an anti-PD-1 antigen binding fragment comprises a VH CHR1 having an amino acid sequence of SEQ ID NO: 80, a VH CHR2 having an amino acid sequence of SEQ ID NO: 81, a VH CHR3 having an amino acid sequence of SEQ ID NO: 82, VL CHR1 having an amino acid sequence of SEQ ID NO: 83, a VL CR2 having an amino acid sequence of SEQ ID NO: 84, and a VL CHR3 having an amino acid sequence of SEQ ID NO: 85. In one instance, an anti-PD-1 polypeptide or an anti-PD-1 antigen binding fragment comprises a VH CHR1 having an amino acid sequence of SEQ ID NO: 86, a VH CR2 having an amino acid sequence of SEQ ID NO: 87, a VH CHR3 having an amino acid sequence of SEQ ID NO: 88, VL CHR1 having an amino acid sequence of SEQ ID NO: 89, a VL CR2 having an amino acid sequence of SEQ ID NO: 90, and a VL CHR3 having an amino acid sequence of SEQ ID NO: 91. In one instance, an anti-PD-1 polypeptide or an anti-PD-1 antigen binding fragment comprises a VH CHR1 having an amino acid sequence of SEQ ID NO: 92, a VH CR2 having an amino acid sequence of SEQ ID NO: 93, a VH CHR3 having an amino acid sequence of SEQ ID NO: 94, VL CHR1 having an amino acid sequence of SEQ ID NO: 95, a VL CR2 having an amino acid sequence of SEQ ID NO: 96, and a VL CHR3 having an amino acid sequence of SEQ ID NO: 97. In one instance, an anti-PD-1 polypeptide or an anti-PD-1 antigen binding fragment comprises a VH CHR1 having an amino acid sequence of SEQ ID NO: 98, a VH CHR2 having an amino acid sequence of SEQ ID NO: 99, a VH CHR3 having an amino acid sequence of SEQ ID NO: 100, VL CHR1 having an amino acid sequence of SEQ ID NO: 101, a VL CHR2 having an amino acid sequence of SEQ ID NO: 102, and a VL CHR3 having an amino acid sequence of SEQ ID NO: 103.

In one instance, an anti-PD-1 polypeptide comprises a fusion protein. Such fusion protein can be, for example, a two-sided Fc fusion protein comprising the extracellular domain (ECD) of programmed cell death 1 (PD-1) and the ECD of tumor necrosis factor (ligand) superfamily member 4 (TNFSF4 or OX40L) fused via hinge-CH2-CH3 Fc domain of human IgG4, expressed in CHO-K1 cells, where the fusion protein has an exemplary amino acid sequence of SEQ ID NO: 104.

### Modification to Fc Region

Disclosed herein are anti-PD-1 polypeptides, wherein the anti-PD-1 polypeptides comprise an Fc region, and the Fc region comprises at least one covalently linked chemical linker. In some embodiments, the chemical linker is covalently attached to an asparagine, glutamine, cysteine, or lysine residue. In some embodiments, the chemical linker is covalently attached to a lysine, or cysteine residue. In some embodiments, the chemical linker is covalently attached to a lysine residue. In some embodiments, the chemical linker is covalently attached to a constant region of the anti-PD-1 polypeptide. In some embodiments, the chemical linker is covalently attached to a constant region of the anti-PD-1 polypeptide.

In some embodiments, the anti-PD-1 polypeptide comprises an Fc region. In some embodiments, the Fc region is an IgG Fc region, an IgA Fc region, an IgD Fc region, an IgM Fc region, or an IgE Fc region. In some embodiments, the Fc region is an IgG Fc region, an IgA Fc region, or an IgD Fc region. In some embodiments, the Fc region is a human Fc region. In some embodiments, the Fc region is a humanized. Fc region. In some embodiments, the Fc region is an IgG Fc region. In some instances, an IgG Fc region is an IgG1 Fc region, an IgG2a Fc region, or an IgG4 Fc region. In some instances, an IgG Fc region is an IgG1 Fc region, an IgG2a Fc region, or an IgG4 Fc region.

One or more mutations may be introduced in an Fc region to reduce Fc-mediated effector functions of an antibody or antigen-binding fragment such as, for example, antibody-dependent cellular cytotoxicity (ADCC) and/or complement function. In some instances, a modified Fc comprises a humanized IgG4 kappa isotype that contains a S228P Fc mutation. In some instances, a modified Fc comprises a human IgG1 kappa where the heavy chain CH₂ domain is engineered with a triple mutation such as, for example: (a) L238P, L239E, and P335S; or (2) K248; K288; and K317.

In some embodiments, the Fc region has an amino acid sequence at least 80%, at least 85%, at least 90%, at least 91%, at least 92%, at least 93%, at least 94%, at least 95%, at least 96%, at least 97%, at least 98%, or at least 99% identical to a sequence as set forth in SEQ ID NO: 105 (Pro Lys Ser Cys Asp Lys Thr His Thr Cys Pro Pro Cys Pro Ala Pro Glu Xaa Xaa Gly Xaa Pro Ser Val Phe Leu Phe Pro Pro Lys Pro Lys Asp Thr Leu Met Ile Ser Arg Thr Pro Glu Val Thr Cys Val Val Val Asp Val Ser His Glu Asp Pro Glu Val Lys Phe Asn Trp Tyr Val Asp Gly Val Glu Val His Asn Ala Lys Thr Lys Pro Arg Glu Glu Gln Tyr Asp Ser Thr Tyr Arg Val Val Ser Val Leu Thr Val Leu His Gln Asp Trp Leu Asn Gly Lys Glu Tyr Lys Cys Lys Val Ser Asn Lys Ala Leu Pro Ala Pro Ile Glu Lys Thr Ile Ser Lys Ala Lys Gly Gln Pro Arg Glu Pro Gln Val Tyr Thr Leu Pro Pro Ser Arg Xaa Glu Xaa Thr Lys Asn Gln Val Ser Leu Thr Cys Leu Val Lys Gly Phe Tyr Pro Ser Asp Ile Ala Val Glu Trp Glu Ser Asn Gly Gln Pro Glu Asn Asn Tyr Lys Xaa Thr Pro Pro Val Leu Asp Ser Asp Gly Ser Phe Phe Leu Tyr Ser Lys Leu Thr Val Asp Lys Ser Arg Trp Gln Gln Gly Asn Val Phe Ser Cys Ser Val Met His Glu Ala Leu His Asn His Tyr Thr Gln Lys Ser Leu Ser Leu Ser Pro Gly, where Xaa can be any naturally occurring amino acid).

In some embodiments, the Fc region comprises one or more mutations which make the Fc region susceptible to modification or conjugation at a particular residue, such as by incorporation of a cysteine residue at a position which does not contain a cysteine in SEQ ID NO: 105. Alternatively, the Fc region could be modified to incorporate a modified natural amino acid or an unnatural amino acid which comprises a conjugation handle, such as one connected to the modified natural amino acid or unnatural amino acid through a linker. In some embodiments, the Fc region does not comprise any mutations which facilitate the attachment of a linker to an additional cytokine (e.g., an IL-2, IL-7, or IL-18 polypeptide). In some embodiments, the chemical linker is attached to a native residue as set forth in SEQ ID NO: 105. In some embodiments, the chemical linker is attached to a native lysine residue of SEQ ID NO: 105.

In some embodiments, the chemical linker can be covalently attached to one amino acid residue of an Fc region of the anti-PD-1 polypeptide. In some embodiments, the chemical linker is covalently attached to a non-terminal residue of the Fc region. In some embodiments, the non-terminal residue is in the CH1, CH2, or CH3 region of the anti-PD-1 polypeptide. In some embodiments, the non-terminal residue is in the CH2 region of the anti-PD-1 polypeptide.

In some embodiments, the chemical linker is attached to the Fc region at an amino acid residue at any one of positions 10-90 of SEQ ID NO: 105. In some embodiments, the chemical linker is attached to the Fc region at an amino acid residue at any one of positions 10-20, 10-30, 10-40, 10-50, 10-60, 10-70, 1-80, 10-90, 10-100, 10-110, 10-120, 10-130, 10-140, 10-150, 10-160, 10-170, 10-180, 10-190, or 10-200 of SEQ ID NO: 105. In some embodiments, the chemical linker is attached to the Fc region at an amino acid residue at any one of positions 20-40, 65-85, or 90-110 of SEQ ID NO: 105. In some embodiments, the chemical linker is attached to the Fc region at an amino acid residue at one of positions 10-30, 50-70, or 80-100 of SEQ ID NO: 105. In some embodiments, the chemical linker is attached to the Fc region at an amino acid residue at one of positions 15-26, 55-65, or 85-90 of SEQ ID NO: 240. In some embodiments, the chemical linker is attached to the Fc region at an amino acid residue at any one of positions 25-35, 70-80, or 95-105 of SEQ ID NO: 105. In some embodiments, the chemical linker is attached to the Fc region at an amino acid residue at any one of positions 30, 32, 72, 74, 79 or 101 of SEQ ID NO: 105. In some embodiments, the chemical linker is attached to the Fc region at an amino acid residue at any one of positions K30, K32, K72, K74, Q79, or K101 of SEQ ID NO: 105. In some embodiments, the chemical linker is attached to the Fc region at amino acid residue 30 of SEQ ID NO: 105. In some embodiments, the chemical linker is attached to the Fc region at amino acid residue 32 of SEQ ID NO: 105. In some embodiments, the chemical linker is attached to the Fc region at amino acid residue 72 of SEQ ID NO: 105. In some embodiments, the chemical linker is attached to the Fc region at amino acid residue 74 of SEQ ID NO: 105. In some embodiments, the chemical linker is attached to the Fc region at amino acid residue 79 of SEQ ID NO: 105. In some embodiments, the chemical linker is attached to the Fc region at amino acid residue 101 of SEQ ID NO: 105.

In some embodiments, the chemical linker is covalently attached at an amino acid residue of the polypeptide which selectively binds a cancer or inflammatory associated antigen (e.g., an anti-PD-1 antibody) such that the function of the polypeptide is maintained (e.g., without denaturing the polypeptide). For example, when the polypeptide is an antibody such as a human IgG (e.g., human IgG1), exposed lysine residues exposed glutamine residues and exposed tyrosine residues are present at the following positions (refer to web site imgt.org/IMGTScientificChart/Numbering/Hu_IGHGnber.html by EU numbering). Exemplary exposed Lysine Residues: CH2 domain (position 246, position 248, position 274, position 288, position 290, position 317, position 320, position 322, and position 338) CH3 domain (position 360, position 414, and position 439). Exemplary exposed Glutamine Residues: CH2 domain (position 295). Exemplary exposed Tyrosine Residues: CH2 domain (position 278, position 296, and position 300) CH3 domain (position 436).

The human IgG, such as human IgG1, may also be modified with a lysine, glutamine, or tyrosine residue at any one of the positions listed above in order provide a residue which is ideally surface exposed for subsequent modification.

In some embodiments, the chemical linker is covalently attached at an amino acid residue in the constant region of an anti-PD-1 antibody. In some embodiments, the chemical linker is covalently attached at an amino acid residue in the CH1, CH2, or CH3 region. In some embodiments, the chemical inker is covalently attached at an amino acid residue in the CH2 region. In some embodiments, the chemical linker may be covalently attached to one residue selected from the following groups of residues following EU numbering in human IgG Fc: amino acid residues 1-478, amino acid residues 2-478, amino acid residues 1-477, amino acid residues 2-477, amino acid residues 10-467, amino acid residues 30-447, amino acid residues 50-427, amino acid residues 100-377, amino acid residues 150-327, amino acid residues 200-327, amino acid residues 240-327, and amino acid residues 240-320.

In some embodiments, the chemical linker is covalently attached to one lysine or glutamine residue of a human IgGFc region. In some embodiments, the chemical linker is covalently attached at Lys 246 of an Fc region of the anti-PD-1 polypeptide, wherein amino acid residue position number is based on Eu numbering. In some embodiments, the chemical linker is covalently attached at Lys 248 of an Fc region of the anti-PD-1 polypeptide, wherein amino acid residue position number is based on Eu numbering. In some embodiments, the chemical linker is covalently attached at Lys 288 of an Fc region of the anti-PD-1 polypeptide, wherein amino acid residue position number is based on Eu numbering. In some embodiments, the chemical linker is covalently attached at Lys 290 of an Fc region of the anti-PD-1 polypeptide, wherein amino acid residue position number is based on Eu numbering. In some embodiments, the chemical linker is covalently attached at Gln 295 of an Fc region of the antibody polypeptide, wherein amino acid residue position number is based on Eu numbering. In some embodiments, the chemical linker is covalently attached at Lys 317 of the anti-PD-1 polypeptide, wherein amino acid residue position number is based on Eu numbering.

In some embodiments, the chemical linker can be covalently attached to an amino acid residue selected from a subset of amino acid residues. In some embodiments, the subset comprises two three, four, five, six, seven, eight, nine, or ten amino acid residues of an Fc region of the anti-PD-1 polypeptide. In some embodiments, the chemical linker can be covalently attached to one of two lysine residues of an Fc region of the anti-PD-1 polypeptide.

In some embodiments, the anti-PD-1 polypeptide will comprise two linkers covalently attached to the Fc region of the anti-PD-1 polypeptide. In some embodiments, each of the two linkers will be covalently attached to a different heavy chain of the anti-PD1 polypeptide. In some embodiments, each of the two linkers will be covalently attached to a different heavy chain of the anti-PD-1 polypeptide at a residue position which is the same. In some embodiments, each of the two linkers will be covalently attached to a different heavy chain of anti-PD-1 polypeptide at a residue position which is different. When the two linkers are covalently attached to residue positions which differ, any combination of the residue positions provided herein may be used in combination.

In some embodiments, a first chemical linker is covalently attached at Lys 248 of a first Fc region of the anti-PD-1 polypeptide, and a second chemical linker is covalently attached at Lys 288 of a second Fc region of the anti-PD-1 polypeptide, wherein residue position number is based on Eu numbering. In some embodiments, a first chemical linker is covalently attached at Lys 246 of a first Fc region of the anti-PD-1 polypeptide, and a second chemical linker is covalently attached at Lys 288 of a second Fc region of the anti-PD-1 polypeptide, wherein residue position number is based on Eu numbering. In some embodiments, a first chemical linker is covalently attached at Lys 248 of a first Fc region of the anti-PD-1 polypeptide, and a second chemical linker is covalently attached at Lys 317 of a second Fc region of the anti-PD-1 polypeptide, wherein residue position number is based on Eu numbering. In some embodiments, a first chemical linker is covalently attached at Lys 246 of a first Fc region of the anti-PD-1 polypeptide, and a second chemical linker is covalently attached at Lys 317 of a second Fc region of the anti-PD-1 polypeptide, wherein residue position number is based on Eu numbering. In some embodiments, a first chemical linker is covalently attached at Lys 288 of a first Fc region of the anti-PD-1 polypeptide, and a second chemical linker is covalently attached at Lys 317 of a second Fc region of the anti-PD-1 polypeptide, wherein residue position number is based on Eu numbering.

### Method of Modifying an Fc Region

Also provided herein are method of preparing a modified Fc region of a polypeptide which selectively binds to PD-1, such as for the attachment of a linker, a conjugation handle, or the cytokine to the polypeptide which selectively binds to PD-1. A variety of methods for site-specific modification of Fc regions of antibodies or other polypeptides which bind to PD-1 are known in the art.

Modification with an Affinity Peptide Configured to Site-Specifically Attach Linker to the Antibody

In some embodiments, an Fc region is modified to incorporate a linker, a conjugation handle, or a combination thereof. In some embodiments, the modification is performed by contacting the Fc region with an affinity peptide bearing a payload configured to attach a linker or other group to the Fc region, such as at a specific residue of the Fc region. In some embodiments, the linker is attached using a reactive group (e.g., a N-hydroxysuccinimide ester) which forms a bond with a residue of the Fc region. In some embodiments, the affinity peptide comprises a cleavable linker. The cleavable linker is configured on the affinity peptide such that after the linker or other group is attached to the Fc region, the affinity peptide can be removed, leaving behind only the desired linker or other group attached to the Fc region. The linker or other group can then be used further to add attach additional groups, such as a cytokine or a linker attached to a cytokine, to the Fc region.

Non-limiting examples of such affinity peptides can be found at least in PCT Publication No. WO2018199337A1, PCT Publication No. WO2019240288A1, PCT Publication No. WO2019240287A1, and PCT Publication No. WO2020090979A1, each of which is incorporated by reference as if set forth herein in its entirety. In some embodiments, the affinity peptide is a peptide which has been modified to deliver the linker/conjugation handle payload one or more specific residues of the Fc region of the antibody. In some embodiments, the affinity peptide has at least 80%, 85%, 90%, 91%, 92%, 93%, 94%, 95%, 96%, 97%, 98%, 99%, or 100% sequence identify to a peptide selected from among (1) QETNPTENLYFQQKNMQCQRRFYEALHDPNLNEEQRNARIRSIRDDDC (SEQ ID NO: 106); (2) QTADNQKNMQCQRRFYEALHDPNLNEEQRNARIRSIRDDCSQSANLLAEAQQLNDAQA PQA (SEQ ID NO: 107); (3) QETKNMQCQRRFYEALHDPNLNEEQRNARIRSIRDDDC (SEQ ID NO: 108); (4) QETFNKQCQRRFYEALHDPNLNEEQRNARIRSIRDDDC (SEQ ID NO: 109); (5) QETFNMQCQRRFYEALHDPNLNKEQRNARIRSIRDDDC (SEQ ID NO: 110); (6) QETFNMQCQRRFYEALHDPNLNEEQRNARIRSIKDDC (SEQ ID NO: 111); (7) QETMQCQRRFYEALHDPNLNEEQRNARIRSIKDDC (SEQ ID NO: 112); (8) QETQCQRRFYEALHDPNLNEEQRNARIRSIKDDC (SEQ ID NO: 113); (9) QETCQRRFYEALHDPNLNEEQRNARIRSIKDDC (SEQ ID NO: 114); (10) QETRGNCAYHKGQLVWCTYH (SEQ ID NO: 115); and (11) QETRGNCAYHKGQIIWCTYH (SEQ ID NO: 116), or a corresponding peptide which has been truncated at the N-terminus by one, two, three, four, or five residues. An exemplary affinity peptide with cleavable linker and conjugation handle payload capable of attaching the payload to residue K248 of an antibody as provided herein is shown below (as reported in Matsuda et al., “Chemical Site-Specific Conjugation Platform to Improve the Pharmacokinetics and Therapeutic Index of Antibody-Drug Conjugates,” Mol. Pharmaceutics 2021, 18, 11, 4058-4066.

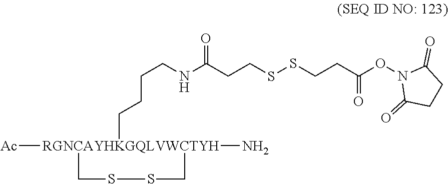

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide a sulfhydryl protecting group as a cleavable portion of the linking group (e.g., the relevant portion of the affinity peptide would have a structure of

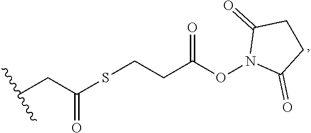 [SMILES: *CC(=O)SCCC(=O)ON1C(=O)CCC1=O]

or another of the cleavable linkers discussed below).

The affinity peptide of the disclosure can comprise a cleavable linker. In some embodiments, the cleavable linker of the affinity peptide connects the affinity peptide to the group which is to be attached to the Fc region and is configured such that the peptide can be cleaved after the group comprising the linker or conjugation handle has been attached. In some embodiments, the cleavable linker is a divalent group. In some embodiments, the cleavable linker can comprise a thioester group, an ester group, a sulfane group; a methanimine group; an oxyvinyl group; a thiopropanoate group; an ethane-1,2-diol group; an (imidazole-1-yl)methan-1-one group; a seleno ether group; a silylether group; a di-oxysilane group; an ether group; a di-oxymethane group; a tetraoxospiro[5.5]undecane group; an acetamidoethyl phosphoramidite group; a bis(methylthio)-pyrazolopyrazole-dione group; a 2-oxo-2-phenylethyl formate group; a 4-oxybenzylcarbamate group; a 2-(4-hydroxy-oxyphenyl)diazinyl)benzoic acid group; a 4-amino-2-(2-amino-2-oxoethyl)-4-oxobut-2-enoic acid group; a 2-(2-methylenehydrazineyl)pyridine group; an N′-methyleneformohydrazide group; or an isopropylcarbamate group, any of which is unsubstituted or substituted. Composition and points of attachment of the cleavable linker to the affinity peptide, as well as related methods of use, are described in, at least, PCT Publication No. WO2018199337A1, PCT Publication No. WO2019240288A1, PCT Publication No. WO2019240287A1, and PCT Publication No. WO2020090979A1.

In some embodiments, the cleavable linker is:

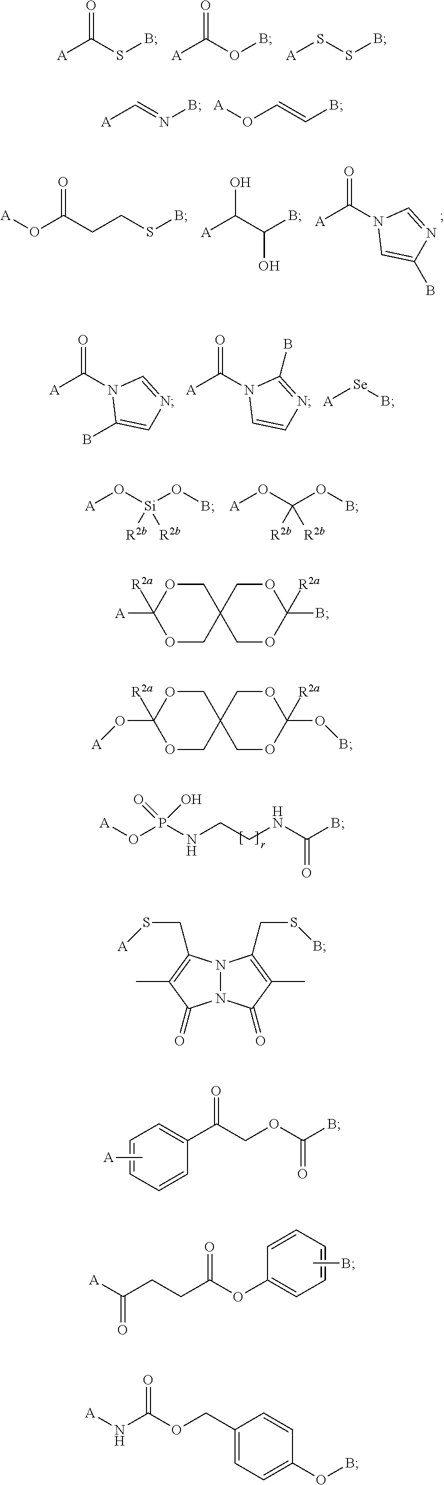

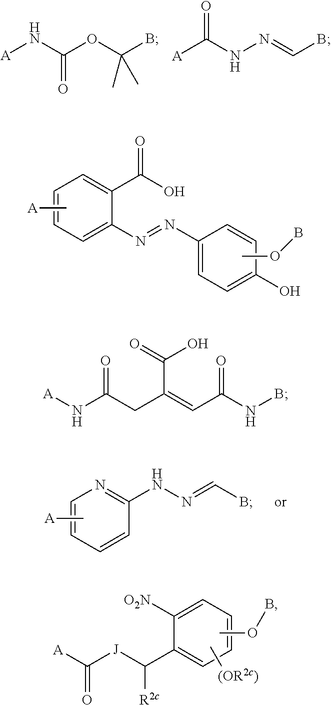

wherein:
one of A or B is a point of attachment the linker and the other of A or B is a point of attachment to the affinity peptide;
each R^{2a} is independently H or optionally substituted alkyl;
each R^{2b} is independently H or optionally substituted alkyl;
R^{2c} is a H or optionally substituted alkyl;
J is a methylene, a N, a S, a Si, or an O atom; and
r is 1,2,3,4,5,6,7,8,9, or 10.

The affinity peptide comprises a reactive group which is configured to enable the covalent attachment of the linker/conjugation handle to the Fc region. In some embodiments, the reactive group is selective for a functional group of a specific amino acid residue, such as a lysine residue, tyrosine residue, serine residue, cysteine residue, or an unnatural amino acid residue of the Fc region incorporated to facilitate the attachment of the linker. The reactive group may be any suitable functional group, such as an activated ester for reaction with a lysine (e.g., N-hydroxysuccinimide ester or a derivate thereof, a pentafluorophenyl ester, etc.) or a sulfhydryl reactive group for reaction with a cysteine (e.g., a Michael acceptor, such as an alpha-beta unsaturated carbonyl or a maleimide). In some embodiments, the reactive group is:

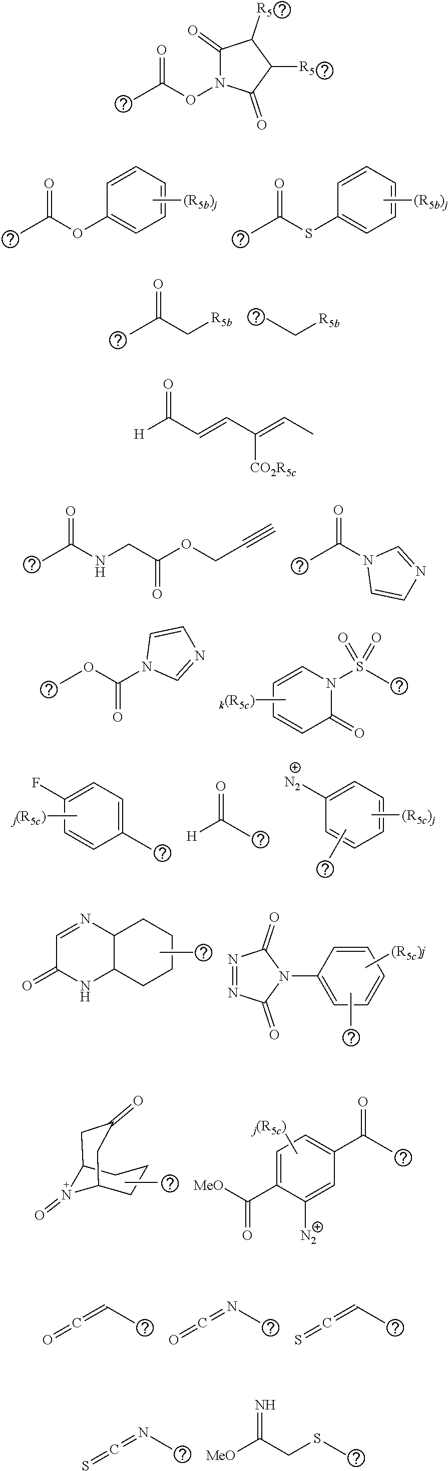

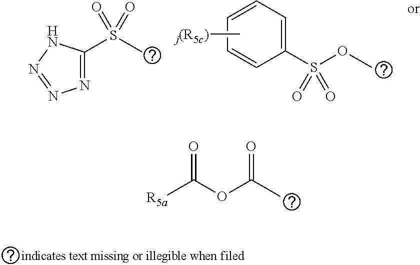

wherein:
each R₅ₐ, R_{5b}, and R_{5c} is independently H, halogen, or optionally substituted alkyl;
   wherein each j is 1, 2, 3, 4, or 5; and
each k is 1, 2, 3, 4, or 5.

In some embodiments, the affinity peptide is used to deliver a reactive moiety to the desired amino acid residue such that the reactive moiety is exposed upon cleavage of the cleavable linker. By way of non-limiting example, the reactive group forms a covalent bond with a desired residue of the Fc region of the polypeptide which selectively binds to anti-PD-1 due to an interaction between the affinity peptide and the Fc region. Following this covalent bond formation, the cleavable linker is cleaved under appropriate conditions to reveal a reactive moiety (e.g., if the cleavable linker comprises a thioester, a free sulfhydryl group is attached to the Fc region following cleavage of the cleavable linker). This new reactive moiety can then be used to subsequently add an additional moiety, such as a conjugation handle, by way of reagent comprising the conjugation handle tethered to a sulfhydryl reactive group (e.g., alpha-halogenated carbonyl group, alpha-beta unsaturated carbonyl group, maleimide group, etc.).

In some embodiments, an affinity peptide is used to deliver a free sulfhydryl group to a lysine of the Fc region. In some embodiments, the free sulfhydryl group is then reacted with a bifunctional linking reagent to attach a new conjugation handle to the Fc region. In some embodiments, the new conjugation handle is then used to form the linker to the attached cytokine. In some embodiments, the new conjugation handle is an alkyne functional group. In some embodiments, the new conjugation handle is a DBCO functional group.

Exemplary bifunctional linking reagents useful for this purpose are of a formula A-B-C, wherein A is the sulfhydryl reactive conjugation handle (e.g., maleimide, α,β-unsaturated carbonyl, a-halogenated carbonyl), B is a lining group, and C is the new conjugation handle (e.g., an alkyne such as DBCO). Specific non-limiting examples of bifunctional linking reagents include

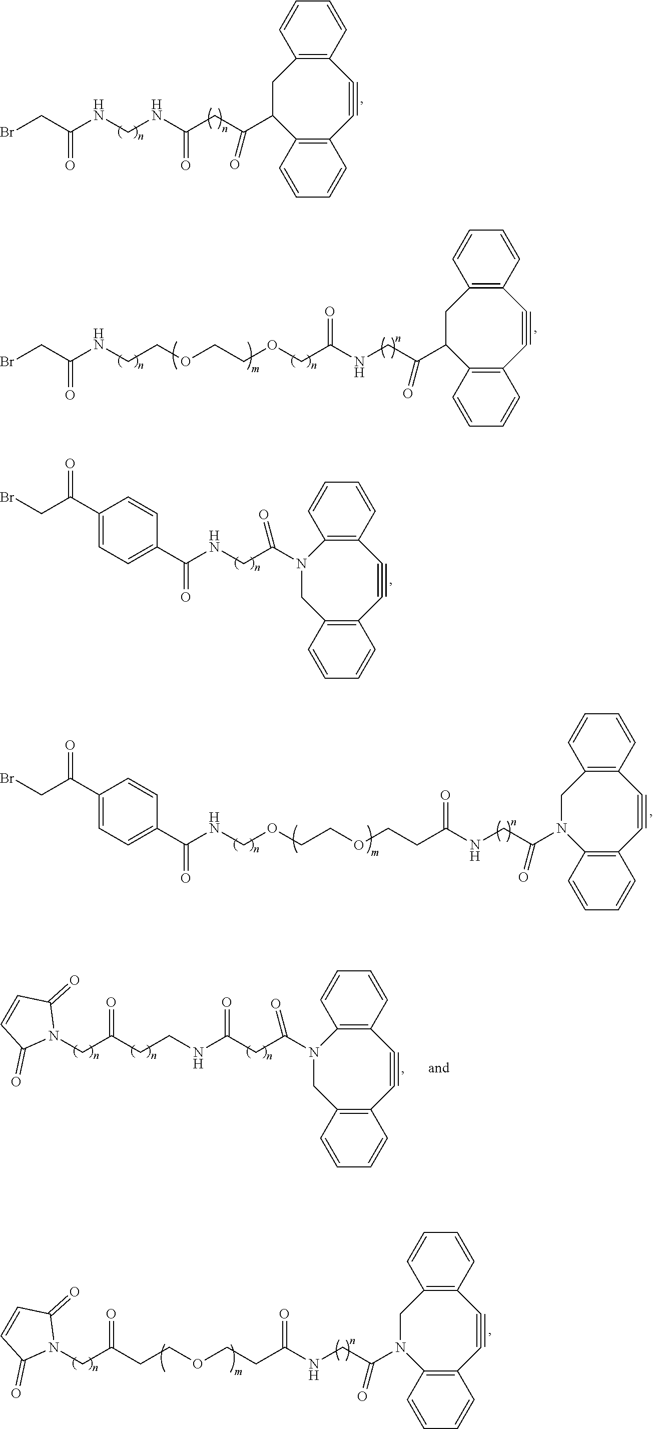

wherein each n is independently an integer from 1-6 and each m is independently an integer from 1-30, and related molecules (e.g., isomers).

Alternatively, the affinity peptide can be configured such that a conjugation handle is added to the Fc region (such as by a linker group) immediately after covalent bond formation between the reactive group and a residue of the Fc region. In such cases, the affinity peptide is cleaved and the conjugation handle is immediately ready for subsequent conjugation to the IL-7 polypeptide.

### Alternative Methods of Attachment—(e.g., Enzyme Mediated)

While the affinity peptide mediated modification of an Fc region of an antibody provided supra possesses many advantages over other methods which can be used to site-specifically modify the Fc region (e.g., ease of use, ability to rapidly generate many different antibody conjugates, ability to use many “off-the-shelf” commercial antibodies without the need to do time consuming protein engineering, etc.), other methods of performing the modification are also contemplated as being within the scope of the present disclosure.

In some embodiments, the present disclosure relates generally to transglutaminase-mediated site-specific antibody-drug conjugates (ADCs) comprising: 1) glutamine-containing tags, endogenous glutamines (e.g., native glutamines without engineering, such as glutamines in variable domains, CDRs, etc.), and/or endogenous glutamines made reactive by antibody engineering or an engineered transglutaminase; and 2) amine donor agents comprising amine donor units, linkers, and agent moieties. Non-limiting examples of such transglutaminase mediated site-specific modifications can be found at least in publications WO2020188061, US2022133904, US2019194641, US2021128743, U.S. Pat. No. 9,764,038, U.S. Ser. No. 10/675,359, U.S. Pat. No. 9,717,803, U.S. Ser. No. 10/434,180, U.S. Pat. No. 9,427,478, which are incorporated by reference as if set forth herein in their entirety.

In another aspect, the disclosure provides an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or different amino acid), and wherein the engineered Fc-containing polypeptide conjugate comprises an amino acid substitution from glutamine to asparagine at position 295 (Q295N; EU numbering scheme).

In some embodiments, the acyl donor glutamine-containing tag is not spatially adjacent to a reactive Lys (e.g., the ability to form a covalent bond as an amine donor in the presence of an acyl donor and a transglutaminase) in the polypeptide or the Fc-containing polypeptide. In some embodiments, the polypeptide or the Fc-containing polypeptide comprises an amino acid modification at the last amino acid position in the carboxyl terminus relative to a wild-type polypeptide at the same position. The amino acid modification can be an amino acid deletion, insertion, substitution, mutation, or any combination thereof.

In some embodiments, the polypeptide conjugate comprises a full length antibody heavy chain and an antibody light chain, wherein the acyl donor glutamine-containing tag is located at the carboxyl terminus of a heavy chain, a light chain, or both the heavy chain and the light chain.

In some embodiments, the polypeptide conjugate comprises an antibody, wherein the antibody is a monoclonal antibody, a polyclonal antibody, a human antibody, a humanized antibody, a chimeric antibody, a bispecific antibody, a minibody, a diabody, or an antibody fragment. In some embodiments, the antibody is an IgG.

In another aspect, described herein is a method for preparing an engineered Fc-containing polypeptide conjugate comprising the formula: (Fc-containing polypeptide-T-A), wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the Fc-containing polypeptide, wherein the acyl donor glutamine-containing tag comprises an amino acid sequence XXQX, wherein X is any amino acid (e.g., X can be the same or a different amino acid), and wherein the engineered Fc-containing polypeptide conjugate comprises an amino acid substitution from glutamine to asparagine at position 295 (Q295N; EU numbering scheme), comprising the steps of: a) providing an engineered (Fc-containing polypeptide)-T molecule comprising the Fc-containing polypeptide and the acyl donor glutamine-containing tag; b) contacting the amine donor agent with the engineered (Fc-containing polypeptide)-T molecule in the presence of a transglutaminase; and c) allowing the engineered (Fc-containing polypeptide)-T to covalently link to the amine donor agent to form the engineered Fc-containing polypeptide conjugate.

In another aspect, described herein is a method for preparing an engineered polypeptide conjugate comprising the formula: polypeptide-T-A, wherein T is an acyl donor glutamine-containing tag engineered at a specific site, wherein A is an amine donor agent, wherein the amine donor agent is site-specifically conjugated to the acyl donor glutamine-containing tag at a carboxyl terminus, an amino terminus, or at an another site in the polypeptide, and wherein the acyl donor glutamine-containing tag comprises an amino acid sequence LLQGPX (SEQ ID NO: 121), wherein X is A or P, or GGLLQGPP (SEQ ID NO: 122), comprising the steps of: a) providing an engineered polypeptide-T molecule comprising the polypeptide and the acyl donor glutamine-containing tag; b) contacting the amine donor agent with the engineered polypeptide-T molecule in the presence of a transglutaminase; and c) allowing the engineered polypeptide-T to covalently link to the amine donor agent to form the engineered Fc-containing polypeptide conjugate.

In some embodiments, the engineered polypeptide conjugate (e.g., the engineered Fc-containing polypeptide conjugate, the engineered Fab-containing polypeptide conjugate, or the engineered antibody conjugate) as described herein has conjugation efficiency of at least about 51%. In another aspect, the invention provides a pharmaceutical composition comprising the engineered polypeptide conjugate as described herein (e.g., the engineered Fc-containing polypeptide conjugate, the engineered Fab-containing polypeptide conjugate, or the engineered antibody conjugate) and a pharmaceutically acceptable excipient.

In some embodiments, described herein is a method for conjugating a moiety of interest (Z) to an antibody, comprising the steps of: (a) providing an antibody having (e.g., within the primary sequence of a constant region) at least one acceptor amino acid residue (e.g., a naturally occurring amino acid) that is reactive with a linking reagent (linker) in the presence of a coupling enzyme, e.g., a transamidase; and (b) reacting said antibody with a linking reagent (e.g., a linker comprising a primary amine) comprising a reactive group (R), optionally a protected reactive group or optionally an unprotected reactive group, in the presence of an enzyme capable of causing the formation of a covalent bond between the acceptor amino acid residue and the linking reagent (other than at the R moiety), under conditions sufficient to obtain an antibody comprising an acceptor amino acid residue linked (covalently) to a reactive group (R) via the linking reagent. Optionally, said acceptor residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

In one aspect, described herein is a method for conjugating a moiety of interest (Z) to an antibody, comprising the steps of: (a) providing an antibody having at least one acceptor glutamine residue; and (b) reacting said antibody with a linker comprising a primary amine (a lysine-based linker) comprising a reactive group (R), preferably a protected reactive group, in the presence of a transglutaminase (TGase), under conditions sufficient to obtain an antibody comprising an acceptor glutamine linked (covalently) to a reactive group (R) via said linker. Optionally, said acceptor glutamine residue of the antibody or antibody fragment is flanked at the +2 position by a non-aspartic acid residue. Optionally, the residue at the +2 position is a non-aspartic acid residue. In one embodiment, the residue at the +2 position is a non-aspartic acid, non-glutamine residue. In one embodiment, the residue at the +2 position is a non-aspartic acid, non-asparagine residue. In one embodiment, the residue at the +2 position is a non-negatively charged amino acid (an amino acid other than an aspartic acid or a glutamic acid). Optionally, the acceptor glutamine is in an Fc domain of an antibody heavy chain, optionally further-within the CH2 domain Optionally, the antibody is free of heavy chain N297-linked glycosylation. Optionally, the acceptor glutamine is at position 295 and the residue at the +2 position is the residue at position 297 (EU index numbering) of an antibody heavy chain.

The antibody comprising an acceptor residue or acceptor glutamine residue linked to a reactive group (R) via a linker comprising a primary amine (a lysine-based linker) can thereafter be reacted with a reaction partner comprising a moiety of interest (Z) to generate an antibody comprising an acceptor residue or acceptor glutamine residue linked to a moiety of interest (Z) via the linker. Thus, in one embodiment, the method further comprises a step (c): reacting (i) an antibody of step b) comprising an acceptor glutamine linked to a reactive group (R) via a linker comprising a primary amine (a lysine-based linker), optionally immobilized on a solid support, with (ii) a compound comprising a moiety of interest (Z) and a reactive group (R′) capable of reacting with reactive group R, under conditions sufficient to obtain an antibody comprising an acceptor glutamine linked to a moiety of interest (Z) via a linker comprising a primary amine (a lysine-based linker). Preferably, said compound comprising a moiety of interest (Z) and a reactive group (R′) capable of reacting with reactive group R is provided at a less than 80 times, 40 times, 20 times, 10 times, 5 times or 4 molar equivalents to the antibody. In one embodiment, the antibody comprises two acceptor glutamines and the compound comprising a moiety of interest (Z) and a reactive group (R′) is provided at 10 or less molar equivalents to the antibody. In one embodiment, the antibody comprises two acceptor glutamines and the compound comprising a moiety of interest (Z) and a reactive group (R′) is provided at 5 or less molar equivalents to the antibody. In one embodiment, the antibody comprises four acceptor glutamines and the compound comprising a moiety of interest (Z) and a reactive group (R′) is provided at 20 or less molar equivalents to the antibody. In one embodiment, the antibody comprises four acceptor glutamines and the compound comprising a moiety of interest (Z) and a reactive group (R′) is provided at 10 or less molar equivalents to the antibody. In one embodiment, steps (b) and/or (c) are carried out in aqueous conditions. Optionally, step (c) comprises: immobilizing a sample of an antibody comprising a functionalized acceptor glutamine residue on a solid support to provide a sample comprising immobilized antibodies, reacting the sample comprising immobilized antibodies with a compound, optionally recovering any unreacted compound and re-introducing such recovered compound to the solid support for reaction with immobilized antibodies, and eluting the antibody conjugates to provide a composition comprising a Z moiety.

### Conjugation Handle Chemistry

In some embodiments, the appropriately modified Fc region of the polypeptide which selectively binds to PD-1 will comprise a conjugation handle which is used to conjugate the polypeptide which selectively binds to PD-1 to an IL-7 polypeptide.

Any suitable reactive group capable of reacting with a complementary reactive group attached to the IL-7 polypeptide can be used as the conjugation handle. In some embodiments, the conjugation handle comprises a reagent for a Cu(I)-catalyzed or “copper-free” alkyne-azide triazole-forming reaction (e.g., strain promoted cycloadditions), the Staudinger ligation, inverse-electron-demand Diels-Alder (IEDDA) reaction, “photo-click” chemistry, tetrazine cycloadditions with trans-cycloctenes, or a metal-mediated process such as olefin metathesis and Suzuki-Miyaura or Sonogashira cross-coupling.

In some embodiments, the conjugation handle comprises a reagent for a “copper-free” alkyne azide triazole-forming reaction. Non-limiting examples of alkynes for said alkyne azide triazole forming reaction include cyclooctyne reagents (e.g., (1R,8S,9s)-Bicyclo[6.1.0]non-4-yn-9-ylmethanol containing reagents, dibenzocyclooctyne-amine reagents, difluorocyclooctynes, or derivatives thereof). In some embodiments, the alkyne functional group is attached to the Fc region. In some embodiments, the azide functional group is attached to the Fc region.

In some embodiments, the conjugation handle comprises a reactive group selected from azide, alkyne, tetrazine, halide, sulfhydryl, disulfide, maleimide, activated ester, alkene, aldehyde, ketone, imine, hydrazine, and hydrazide. In some embodiments, the IL-7 polypeptide comprises a reactive group complementary to the conjugation handle of the Fc region. In some embodiments, the conjugation handle and the complementary conjugation handle comprise “CLICK” chemistry reagents. Exemplary groups of click chemistry residue are shown in Hein et al., “Click Chemistry, A Powerful Tool for Pharmaceutical Sciences,” Pharmaceutical Research volume 25, pages 2216-2230 (2008); Thirumurugan et al., “Click Chemistry for Drug Development and Diverse Chemical-Biology Applications,” Chem. Rev. 2013, 113, 7, 4905-4979; US20160107999A1; U.S. Ser. No. 10/266,502B2; and US20190204330A1, each of which is incorporated by reference in its entirety.

### Linker Structure

In some embodiments, the linker used to attach the polypeptide which selectively binds to PD-1 and the cytokine (such as the IL-7 polypeptide) comprises points of attachment at both moieties. The points of attachment can be any of the residues for facilitating the attachment as provided herein. The linker structure can be any suitable structure for creating the spatial attachment between the two moieties. In some embodiments, the linker provides covalent attachment of both moieties. In some embodiments, the linker is a chemical linker (e.g., not an expressed polypeptide as in a fusion protein).

In some embodiments, the linker comprises a polymer. In some embodiments, the linker comprises a water-soluble polymer. In some embodiments, the linker comprises poly(alkylene oxide), polysaccharide, poly(vinyl pyrrolidone), poly(vinyl alcohol), polyoxazoline, poly(acryloylmorpholine), or a combination thereof. In some embodiments, the linker comprises poly(alkylene oxide). In some embodiments, the poly(alkylene oxide) is polyethylene glycol or polypropylene glycol, or a combination thereof. In some embodiments, the poly(alkylene oxide) is polyethylene glycol.

In some embodiments, the linker is a bifunctional linker. In some embodiments, the bifunctional linker comprises an amide group, an ester group, an ether group, a thioether group, or a carbonyl group. In some embodiments, the linker comprises a non-polymer linker. In some embodiments, the linker comprises a non-polymer, bifunctional linker. In some embodiments, the non-polymer, bifunctional linker comprises succinimidyl 4-(N-maleimidomethyl)cyclohexane-1-carboxylate; Maleimidocaproyl; Valine-citrulline; Allyl(4-methoxyphenyl)dimethylsilane; 6-(Allyloxycarbonylamino)-1-hexanol; 4-Aminobutyraldehyde diethyl acetal; or (E)-N-(2-Aminoethyl)-4-{2-[4-(3-azidopropoxy)phenyl]diazenyl}benzamide hydrochloride.

The linker can be branched or linear. In some embodiments, the linker is linear. In some embodiments, the linker is branched. In some embodiments, the linker comprises a linear portion (e.g., between the first point of attachment and the second point of attachment) of a chain of at least 10, 20, 50, 100, 500, 1000, 2000, 3000, or 5000 atoms. In some embodiments, the linker comprises a linear portion of a chain of at least 10, 20, 30, 40, or 50 atoms. In some embodiments, the linker comprises a linear portion of at least 10 atoms. In some embodiments, the linker comprises a liner portion of a chain of at most 30, 40, 50, 60, 70, 80, 90, or 100 atoms. In some embodiments, the linker is branched and comprises a linear portion of a chain of at least 10, 20, 50, 100, 500, 1000, 2000, 3000, or 5000 atoms. In some embodiments, the linker comprises a linear portion of a chain of at most about 300, 250, 200, 150, 100, or 50 atoms.

In some embodiments, the linker has a molecular weight of about 200 Daltons to about 2000 Daltons. In some embodiments, the linker has a molecular weight of about 200 Daltons to about 5000 Daltons. In some embodiments, the linker has a molecular weight of 200 Daltons to 100,000 Daltons. In some embodiments, the linker has a molecular weight of 200 Daltons to 500 Daltons, 200 Daltons to 750 Daltons, 200 Daltons to 1,000 Daltons, 200 Daltons to 5,000 Daltons, 200 Daltons to 10,000 Daltons, 200 Daltons to 20,000 Daltons, 200 Daltons to 50,000 Daltons, 200 Daltons to 100,000 Daltons, 500 Daltons to 750 Daltons, 500 Daltons to 1,000 Daltons, 500 Daltons to 5,000 Daltons, 500 Daltons to 10,000 Daltons, 500 Daltons to 20,000 Daltons, 500 Daltons to 50,000 Daltons, 500 Daltons to 100,000 Daltons, 750 Daltons to 1,000 Daltons, 750 Daltons to 5,000 Daltons, 750 Daltons to 10,000 Daltons, 750 Daltons to 20,000 Daltons, 750 Daltons to 50,000 Daltons, 750 Daltons to 100,000 Daltons, 1,000 Daltons to 5,000 Daltons, 1,000 Daltons to 10,000 Daltons, 1,000 Daltons to 20,000 Daltons, 1,000 Daltons to 50,000 Daltons, 1,000 Daltons to 100,000 Daltons, 5,000 Daltons to 10,000 Daltons, 5,000 Daltons to 20,000 Daltons, 5,000 Daltons to 50,000 Daltons, 5,000 Daltons to 100,000 Daltons, 10,000 Daltons to 20,000 Daltons, 10,000 Daltons to 50,000 Daltons, 10,000 Daltons to 100,000 Daltons, 20,000 Daltons to 50,000 Daltons, 20,000 Daltons to 100,000 Daltons, or 50,000 Daltons to 100,000 Daltons. In some embodiments, the linker has a molecular weight of 200 Daltons, 500 Daltons, 750 Daltons, 1,000 Daltons, 5,000 Daltons, 10,000 Daltons, 20,000 Daltons, 50,000 Daltons, or 100,000 Daltons. In some embodiments, the linker has a molecular weight of at least 200 Daltons, 500 Daltons, 750 Daltons, 1,000 Daltons, 5,000 Daltons, 10,000 Daltons, 20,000 Daltons, or 50,000 Daltons. In some embodiments, the linker has a molecular weight of at most 500 Daltons, 750 Daltons, 1,000 Daltons, 5,000 Daltons, 10,000 Daltons, 20,000 Daltons, 50,000 Daltons, or 100,000 Daltons. In a preferred embodiments, the linker has a molecular weight of less than 5000 Daltons, less than 4000 Daltons, less than 3000 Daltons, or less than 2000 Daltons, and the linker is monodisperse (e.g., for a population of conjugate compositions herein, there is a high degree of uniformity of the linker structure between the polypeptide which binds specifically to PD-1 and the IL-7 polypeptide).

In some embodiments, the linker comprises a reaction product one or more pairs of conjugation handles, and a complementary conjugation handle thereof. In some embodiments, the reaction product comprises a triazole, a hydrazone, pyridazine, a sulfide, a disulfide, an amide, an ester, an ether, an oxime, an alkene, or any combination thereof. In some embodiments, the reaction product comprises a triazole. The reaction product can be separated from the first point of attachment and the second point of attachment by any portion of the linker. In some embodiments, the reaction product is substantially in the center of the linker. In some embodiments, the reaction product is substantially closer to one point of attachment than the other.

In some embodiments, the linker comprises a structure of Formula (X)

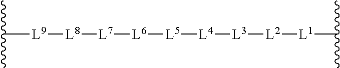

wherein each of L¹, L², L³, L⁴, L⁵, L⁶, L⁸, and L⁹ is independently —O—, —NR^{L}—, —N(R^{L})₂⁺—, —OP(═O)(OR^{L})O—, —S—, —S(═O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₆ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
   wherein each

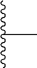

is a point of attachment to the polypeptide which selectively binds to PD-1 or the cytokine (e.g., the IL-7 polypeptide).

In some embodiments, the linker comprises a structure of Formula (X^{a})

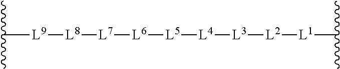

wherein each of L¹, L², L³, L⁴, L⁵, L⁶, L⁷, L⁸, and L⁹ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂+(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(C₁-C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₆ cycloalkyl, substituted or unsubstituted C₂-C₇ heterocycloalkyl, substituted or unsubstituted aryl, or substituted or unsubstituted heteroaryl; and
each of qa, qb, qc and qd is independently an integer from 1-100,
wherein each

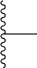

is a point of attachment to the polypeptide which selectively binds to PD-1 or the cytokine (e.g., the IL-7 polypeptide).

In some embodiments, the linker comprises a structure of Formula (X′)

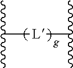

wherein each L′ is independently —O—, —NR^{L}—, —(C₁-C₆ alkylene)NR^{L}—, —NR^{L}(C₁-C₆ alkylene)-, —N(R^{L})₂⁺—, —(C₁-C₆ alkylene)N(R^{L})₂⁺—, —N(R^{L})₂⁺—(C₁-C₆ alkylene)-, —OP(═O)(OR^{L})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O) (C₁-C₆ alkylene)-, —C(═O)O—, —OC(═O)—, —OC(═O)O—, —C(═O)NR^{L}—, —C(═O)NR^{L}(—C₆ alkylene)-, —(C₁-C₆ alkylene)C(═O)NR^{L}—, —NR^{L}C(═O)—, —(C₁-C₆ alkylene)NR^{L}C(═O)—, —NR^{L}C(═O)(C₁-C₆ alkylene)-, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, a reaction product of a conjugation handle and a complementary conjugation handle, or absent;
each R^{L} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₆ cycloalkyl, substituted or unsubstituted C₂-C₇ heterocycloalkyl, substituted or unsubstituted aryl, or substituted or unsubstituted heteroaryl;
each of qa, qb, qc and qd is independently an integer from 1-100; and
g is an integer from 1-100,
   wherein each

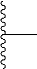

is a point of attachment to the IL-7 polypeptide or the antibody or antigen binding fragment.

In some embodiments, the linker of Formula (X) or of Formula (X^{a}) or of Formula (X′) comprises the structure:

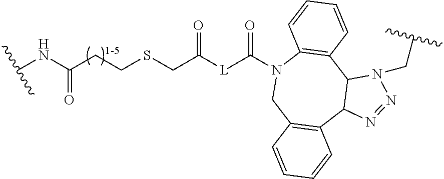

wherein

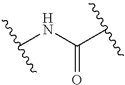 [SMILES: *NC(*)=O]

is the first point of attachment to a lysine residue of the polypeptide which selectively binds to PD-1;
L is a linking group; and

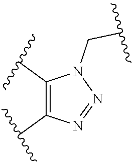 [SMILES: *Cn1nnc(*)c1*]

is a point of attachment to a linking group which connects to the first point of attachment,
or a regioisomer thereof.

In some embodiments, L has a structure

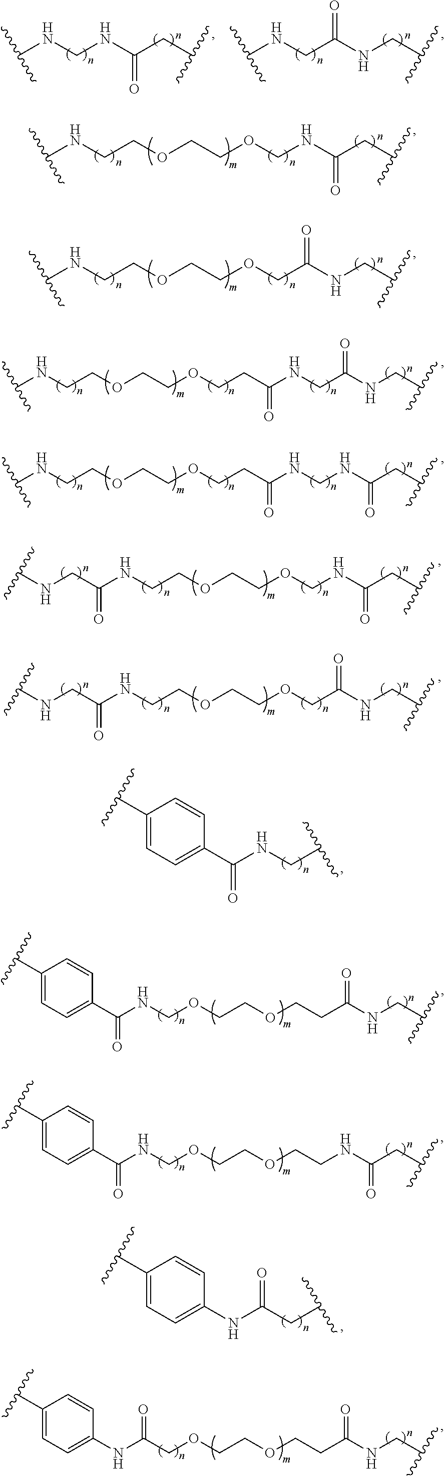

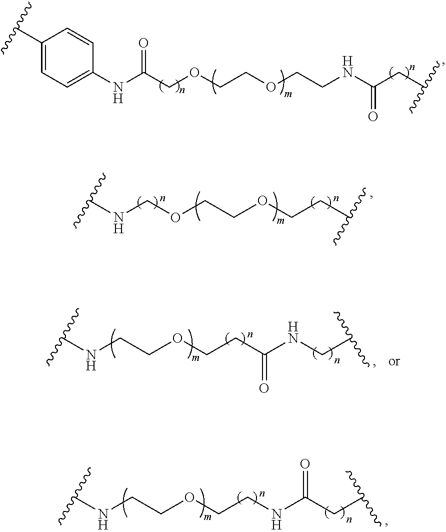

wherein each n is independently an integer from 1-6 and each m is an integer from 1-30. In some embodiments, each m is independently 2 or 3. In some embodiments, each m is an integer from 1-24, from 1-18, from 1-12, or from 1-6.

In some embodiments, the linker of Formula (X) or of Formula (X^{a}) or of Formula (X′) comprises the structure:

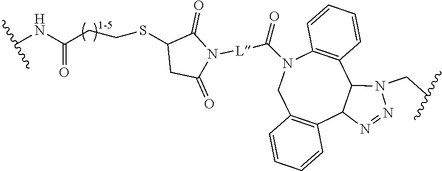

wherein

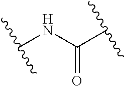 [SMILES: *NC(*)=O]

is the first point of attachment to a lysine residue of the polypeptide which selectively binds to PD-1;
L″ is a linking group; and

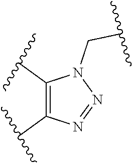 [SMILES: *Cn1nnc(*)c1*]

is a point of attachment to a linking group which connects to the first point of attachment,
or a regioisomer thereof.

In some embodiments, L″ has a structure

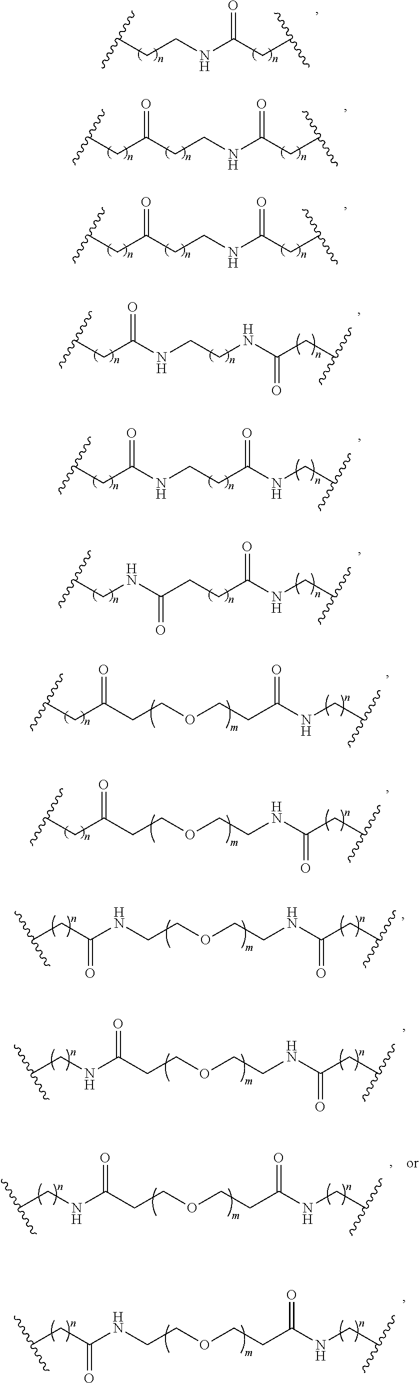

wherein each n is independently an integer from 1-6 and each m is independently an integer from 1-30. In some embodiments, each m is independently 2 or 3. In some embodiments, each m is an integer from 1-24, from 1-18, from 1-12, or from 1-6.

In some embodiments, L or L″ comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more subunits each independently selected from

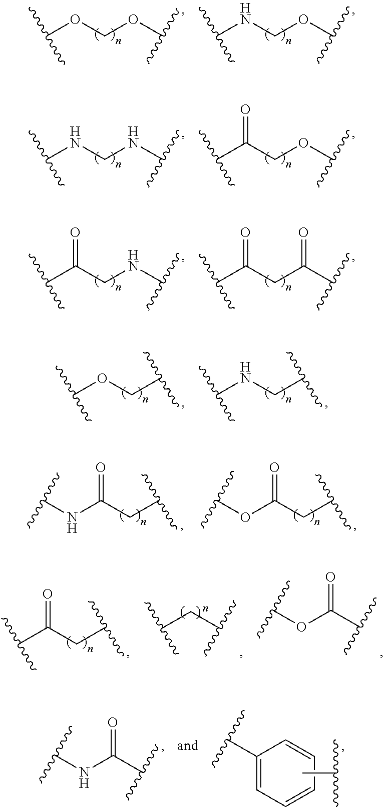

wherein each n is independently an integer from 1-30. In some embodiments, each n is independently an integer from 1-6. In some embodiments, L or L″ comprise 1, 2, 3, 4, 5, 6, 7, 8, 9, or 10 of the subunits.

In some embodiments, L or L″ is a structure of Formula (X″)

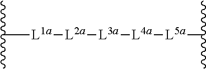

wherein each of L^{1a}, L^{2a}, L^{3a}, L^{4a}, L^{5a}, is independently —O—, —NR^{La}—, —(C₁-C₆ alkylene)NR^{La}—, —NR^{La}(C₁-C₆ alkylene)-, —N(R^{L})₂⁺—, —(C₁-C₆ alkylene)N(R^{La})+(C₁-C₆ alkylene)-, —N(R^{L})₂⁺—, —OP(═O)(OR^{La})O—, —S—, —(C₁-C₆ alkylene)S—, —S(C₁-C₆ alkylene)-, —S(═O)—, —S(═O)₂—, —C(═O)—, —(C₁-C₆ alkylene)C(═O)—, —C(═O)(C₁-C₆ alkylene)-, —C(═O)O—, —OC(═O)—, —OC(═O)O—, —C(═O)NR^{La}—, —C(═O)NR^{La}(C₁-C₆ alkylene)-, —(C₁-C₆ alkylene)C(═O)NR^{La}—, —NR^{La}C(O)—, —(C₁-C₆ alkylene)NR^{La}C(═O)—, —NR^{La}C(═O)(C₁-C₆ alkylene)-, —OC(═O)NR^{La}—, —NR^{La}C(═O)O—, —NR^{La}C(═O)NR^{La}—, —NR^{La}C(═S)NR^{La}—, —CR^{La}═N—, —N═CR^{La}, —NR^{La}S(═O)₂—, —S(═O)₂NR^{La}—, —C(═O)NR^{La}S(═O)₂—, —S(═O)₂NR^{La}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qe}—, —(O—CH₂—CH₂)_{qf}—, —(CH₂—CH(CH₃)—O)_{qg}—, —(O—CH(CH₃)—CH₂)_{qh}—, a reaction product of a conjugation handle and a complementary conjugation handle, or absent; (C₁-C₆ alkylene)
each R^{La} is independently hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₆ cycloalkyl, substituted or unsubstituted C₂-C₇ heterocycloalkyl, substituted or unsubstituted aryl, or substituted or unsubstituted heteroaryl; and
each of qe, qf, qg and qh is independently an integer from 1-100.

In some embodiments, L or L″ comprises a linear chain of 2 to 10, 2 to 15, 2 to 20, 2 to 25, or 2 to 30 atoms. In some embodiments, the linear chain comprises one or more alkyl groups (e.g., lower alkyl (C₁-C₄)), one or more aromatic groups (e.g., phenyl), one or more amide groups, one or more ether groups, one or more ester groups, or any combination thereof.

In some embodiments, the linking group which connects to the first point of attachment (e.g., the point of attachment to the cytokine) comprises poly(ethylene glycol). In some embodiments, the linking group comprises about 2 to about 30 poly(ethylene glycol) units. In some embodiments, the linking group which connects to the first point of attachment (e.g., the point of attachment to the cytokine) is a functionality attached to a cytokine provided herein which comprises an azide (e.g., the triazole is the reaction product of the azide).

In some embodiments, L is —O—, —NR^{L}—, —N(R^{L})₂⁺—, —OP(═O)(OR^{L})O—, —S—, —S(O)—, —S(═O)₂—, —C(═O)—, —C(═O)O—, —OC(═O)—, —OC(═O)O—, —C(═O)NR^{L}—, —NR^{L}C(═O)—, —OC(═O)NR^{L}—, —NR^{L}C(═O)O—, —NR^{L}C(═O)NR^{L}—, —NR^{L}C(═S)NR^{L}—, —CR^{L}═N—, —N═CR^{L}, —NR^{L}S(═O)₂—, —S(═O)₂NR^{L}—, —C(═O)NR^{L}S(═O)₂—, —S(═O)₂NR^{L}C(═O)—, substituted or unsubstituted C₁-C₆ alkylene, substituted or unsubstituted C₁-C₆ heteroalkylene, substituted or unsubstituted C₂-C₆ alkenylene, substituted or unsubstituted C₂-C₆ alkynylene, substituted or unsubstituted C₆-C₂₀ arylene, substituted or unsubstituted C₂-C₂₀ heteroarylene, —(CH₂—CH₂—O)_{qa}—, —(O—CH₂—CH₂)_{qb}—, —(CH₂—CH(CH₃)—O)_{qc}—, —(O—CH(CH₃)—CH₂)_{qa}—, wherein R^{L} hydrogen, substituted or unsubstituted C₁-C₄ alkyl, substituted or unsubstituted C₁-C₄ heteroalkyl, substituted or unsubstituted C₂-C₆ alkenyl, substituted or unsubstituted C₂-C₅ alkynyl, substituted or unsubstituted C₃-C₆ cycloalkyl, substituted or unsubstituted C₂-C₇ heterocycloalkyl, substituted or unsubstituted aryl, or substituted or unsubstituted heteroaryl; and each of qa, qb, qc and qd is independently an integer from 1-100.

In some embodiments, the linking group which connects to the first point of attachment (e.g., the point of attachment to the cytokine) comprises poly(ethylene glycol). In some embodiments, the linking group comprises about 2 to about 30 poly(ethylene glycol) units.

In some embodiments, each reaction product of a conjugation handle and a complementary conjugation handle independently comprises a triazole, a hydrazone, pyridazine, a sulfide, a disulfide, an amide, an ester, an ether, an oxime, or an alkene. In some embodiments, each reaction product of a conjugation handle and a complementary conjugation handle comprises a triazole. In some embodiments, each reaction product of a conjugation handle and a complementary conjugation handle comprise a structure of

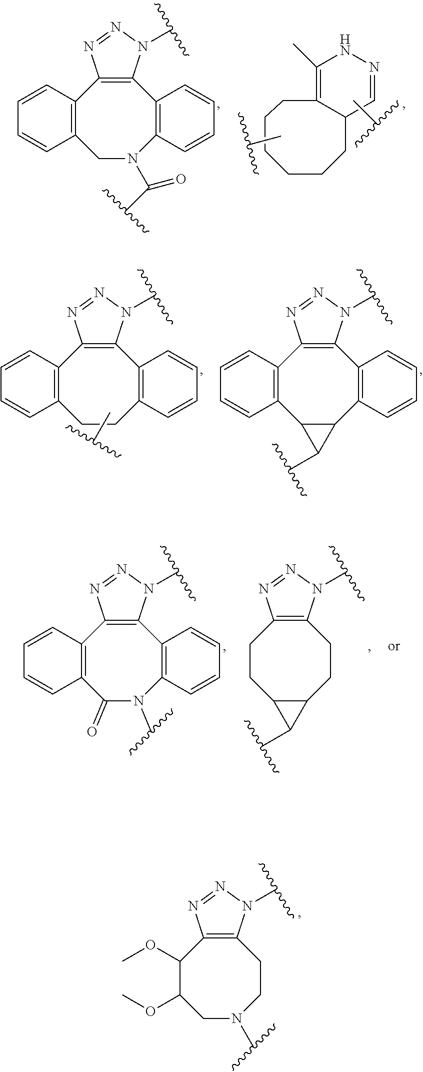

or a regioisomer or derivative thereof.

In some embodiments, the linker is a cleavable linker. In some embodiments, the cleavable linker is cleaved at, near, or in a tumor microenvironment. In some embodiments, the tumor is mechanically or physically cleaved at, near, or in the tumor microenvironment. In some embodiments, the tumor is chemically cleaved at, near, or in a tumor microenvironment. In some embodiments, the cleavable linker is a reduction sensitive linker. In some embodiments, the cleavable linker is an oxidation sensitive linker. In some embodiments, the cleavable linker is cleaved as a result of pH at, near, or in the tumor microenvironment. In some embodiments, the linker by a tumor metabolite at, near, or in the tumor microenvironment. In some embodiments, the cleavable linker is cleaved by a protease at, near, or in the tumor microenvironment.

### IL-7 Polypeptides

Cytokines are proteins produced in the body that are important in cell signaling. Cytokines can modulate the immune system, and cytokine therapy utilizes the immunomodulatory properties of the molecules to enhance the immune system of a subject and kills cancer cells.

Interleukin 7 (IL-7) is a non-hematopoietic cell-derived cytokine with a central role in the adaptive immune system. IL-7 promotes lymphocyte development in the thymus and maintains survival of naïve and memory T cell homeostasis in the periphery. IL-7 is secreted by stromal cells in the bone marrow and thymus, and is also produced by keratinocytes, dendritic cells, hepatocytes, neurons, and epithelial cells. IL-7 is not produced by normal lymphocytes. The autocrine production of IL-7 cytokine mediated by T-cell acute lymphoblastic leukemia (T-ALL) can be involved in the oncogenic development of T-ALL. -IL-7 is important for the organogenesis of lymph nodes and for the maintenance of activated T cells recruited into the secondary lymphoid organs.

Interleukin-7 (IL-7) belongs to the γ-chain family of cytokines, which also includes IL-2, IL-4, IL-9, IL-15, and IL-21. Like all receptors of the γ-chain cytokine family, the IL-7 receptor utilizes the common γ-chain subunit (CD132) in conjunction with another subunit specific for IL-7 named IL-7 receptor alpha subunit (IL-7Rα, a.k.a. CD127). Thus, the IL-7 receptor (IL-7R) is a heterodimer of the IL-7a subunit and the common γ-chain.

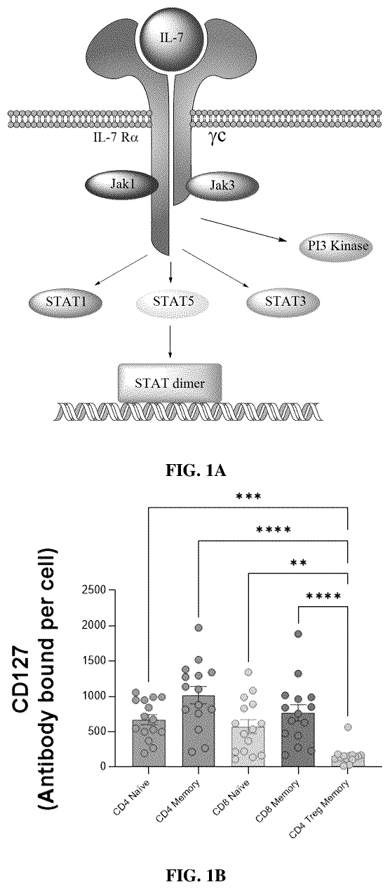

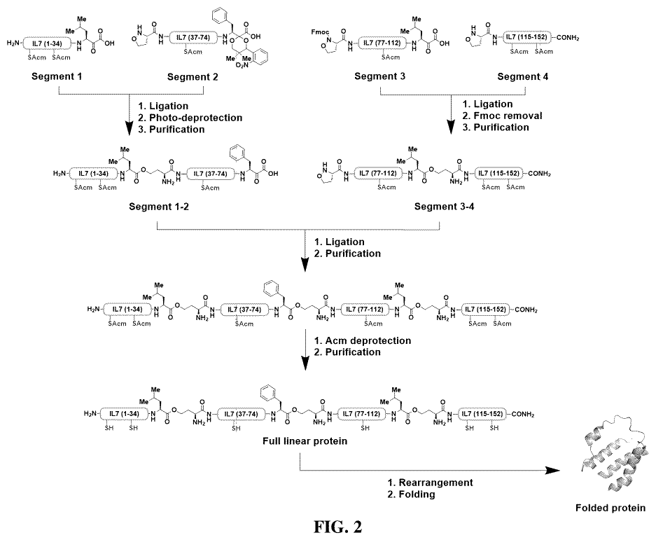

FIG. 1A illustrates the mechanism of action of IL-7 signaling, particularly the signallinc cascade induced by IL-7 binding to its receptor. T cell-mediated immunomodulation can be defined as altering the T_{reg}:T_{eff} ratio. Immunosuppression skews the net T_{reg}:T_{eff} ratio towards the ‘tolerogenic’ Treg component, while immunoactivation skews the response toward the ‘proinflammatory’ T_{eff} component. In the treatment of autoimmune diseases, achieving an immunosuppressive state is desirable to prevent ongoing injury by activated T_{eff} cells. In contrast, an innate or induced immunosuppressive state can prevent pathogen-induced disease while allowing for the progression of cancer. Methods of attenuating an existing endogenous immunosuppressive state that prevents effective T cell-mediated immunorecognition of cancer cells can be used to modulate T_{reg}:T_{eff} ratios to treat autoimmune diseases and cancers.

The IL-7R α/γ heterodimer (IL-7R) is expressed on T cells, pre-B cells, and dendritic cells. Because IL-7R is expressed across immune T cell subtypes, IL-7 can act as a “pan-T cell” cytokine, activating numerous effector T (T_{eff}) and regulatory T (T_{reg}) cells (e.g., CD8 Naïve, CD4 Naïve, CD8 memory, CD4 memory, and CD4 Treg cells) with nearly identical potency. This is in contrast to other cytokines such as IL-2, which is known to be a strong activator of T_{reg} cells Conversely, IL-7 shows nearly identical potency for T_{eff} and T_{reg} cells.

Although many T cell subtypes express IL-7R, different subtypes express IL-7R at different levels. In particular, many T_{eff} subtypes express IL-7R at significantly higher levels than T_{reg} cells. This is shown in FIG. 1B, which shows the amount of anti-CD127 antibody bound per cell (Y-axis) across a number of T-cell subtypes (x-axis). Notably, the T_{eff} subtypes (CD4 Naïve, CD4 Memory, CD8 Naïve, and CD8 Memory) all expressed significantly more IL-7R than the T_{reg} subtype (CD4 T_{reg} Memory).

IL-7 binds to the IL-7 receptor (IL-7R), a heterodimer consisting of IL-7R alpha (IL-7Rα) and common gamma chain receptor. Binding results in a cascade of signals important for T-cell development within the thymus and survival within the periphery, Knockout mice that genetically lack IL-7R exhibit thymic atrophy, arrested T-cell development at the double positive stage, and severe lymphopenia.

In mouse models, IL-7 has demonstrated anti-cancer effects. However, use of IL-7 can lead to systemic toxicity. Conjugation of IL-7 to an anti-PD-1 polypeptide of the disclosure can improve IL-7 polypeptide selectivity, enhance the therapeutic potential of IL-7, and minimize the risk of side effects from administering IL-7 therapies.

The present disclosure describes anti-PD-1 polypeptides conjugated to interleukin-7 (IL-7) polypeptides and their use as therapeutic agents. IL-7 polypeptides provided herein can be used as immunotherapies or as parts of other immunotherapy regimens.

An IL-7 polypeptide attached to the polypeptide which binds specifically to PD-1 can be any of the IL-7 polypeptides described herein (including any of the synthetic IL-7 polypeptides described herein). In some embodiments, an IL-7 polypeptide provided herein linked to an anti-PD-1 polypeptide comprises an amino acid sequence having at least about 80% sequence identity to the sequence set forth in SEQ ID NO: 1. In some embodiments, the IL-7 polypeptide comprises an amino acid sequence having at least about 85% sequence identity to the sequence set forth in SEQ ID NO: 1. In some embodiments, the IL-7 polypeptide comprises an amino acid sequence having at least about 90% sequence identity to the sequence set forth in SEQ ID NO: 1. In some embodiments, the IL-7 polypeptide comprises an amino acid sequence having at least about 95% sequence identity to the sequence set forth in SEQ ID NO: 1. In some embodiments, the IL-7 polypeptide provided herein comprises an amino acid sequence having at least about 96% sequence identity to the sequence set forth in SEQ ID NO: 1. In some embodiments, the IL-7 polypeptide provided herein comprises an amino acid sequence having at least about 97% sequence identity to the sequence set forth in SEQ ID NO: 1. In some embodiments, the IL-7 polypeptide provided herein comprises an amino acid sequence having at least about 98% sequence identity to the sequence set forth in SEQ ID NO: 1. In some embodiments, the IL-7 polypeptide provided herein comprises an amino acid sequence having at least about 99% sequence identity to the sequence set forth in SEQ ID NO: 1. In some embodiments, the IL-7 polypeptide provided herein comprises an amino acid sequence identical to the sequence set forth in SEQ ID NO: 1.

In some embodiments, the IL-7 polypeptide linked to the polypeptide which selectively binds to PD-1 (e.g., the anti-PD-1 antibody) is a synthetic IL-7 polypeptide. The synthetic polypeptide linked to the polypeptide can be any of the synthetic IL-7 polypeptides provided herein. In some embodiments, the synthetic IL-7 polypeptide comprises an amino acid sequence having at least 80% sequence identity to the sequence set forth in SEQ ID NO: 3. In some embodiments, the synthetic IL-7 polypeptide comprises an amino acid sequence having at least 85% sequence identity to the sequence set forth in SEQ ID NO: 3. In some embodiments, the synthetic IL-7 polypeptide comprises an amino acid sequence having at least 90% sequence identity to the sequence set forth in SEQ ID NO: 3. In some embodiments, the synthetic IL-7 polypeptide comprises an amino acid sequence having at least 95% sequence identity to the sequence set forth in SEQ ID NO: 3. In some embodiments, the synthetic IL-7 polypeptide comprises an amino acid sequence having at least 96% sequence identity to the sequence set forth in SEQ ID NO: 3. In some embodiments, the synthetic IL-7 polypeptide comprises an amino acid sequence having at least 97% sequence identity to the sequence set forth in SEQ ID NO: 3. In some embodiments, the synthetic IL-7 polypeptide comprises an amino acid sequence having at least 98% sequence identity to the sequence set forth in SEQ ID NO: 3. In some embodiments, the synthetic IL-7 polypeptide comprises an amino acid sequence having at least 99% sequence identity to the sequence set forth in SEQ ID NO: 3. In some embodiments, the synthetic IL-7 polypeptide comprises an amino acid sequence identical to the sequence set forth in SEQ ID NO: 3.

### Synthetic IL-7 Polypeptides

In one aspect, disclosed herein is a synthetic IL-7 polypeptide. In some embodiments, the synthetic IL-7 polypeptide is prepared from one or more chemically synthesized peptides. In some embodiments, the synthetic IL-7 polypeptide is synthesized from one or more chemically synthesized precursor fragments. In some embodiments, the synthetic IL-7 polypeptide is prepared from one or more chemically synthesized precursor fragments that are ligated together to produce the full-length synthetic IL-7 polypeptide. In some embodiments, a synthetic IL-7 polypeptide as provided herein is incorporated into an immunocytokine composition (e.g., is attached via a linker) with a polypeptide which binds specifically to PD-1 (e.g., an anti-PD-1 antibody). In some embodiments, a synthetic IL-7 polypeptide as provided herein is attached via a linker to an additional moiety, such as a polymer or an antibody or antigen binding fragment thereof.

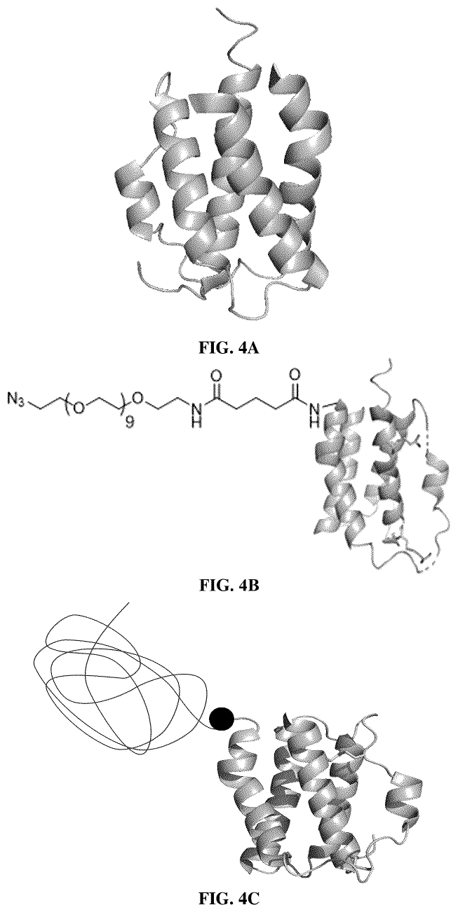

The synthetic IL-7 polypeptide can comprise any of the point of attachments provided herein for an IL-7 polypeptide linked with another polypeptide, such as a polypeptide which binds specifically to PD-1 (e.g., any of the points of attachment discussed supra, such as the N-terminal residue). In some embodiments, a synthetic IL-7 polypeptide exhibits a similar or substantially identical activity to a corresponding recombinant IL-7 (e.g., an IL-7 having the same functional modifications to the structure or sequence of the IL-7 polypeptide). In some embodiments, the synthetic IL-7 polypeptide adopts a tertiary structure similar or substantially identical to that of wild type IL-7 (e.g., the conformation shown in FIG. 4A, which shows a 3D representation of a properly folded IL-7).

In some embodiments, the synthetic IL-7 polypeptide is prepared from one or more chemically synthesized fragments. In some embodiments, the synthetic IL-7 polypeptide is prepared from 1, 2, 3, 4, 5, 6, 7, 8, or more chemically synthesized fragments. In some embodiments, the synthetic IL-7 polypeptide is prepared from 4 chemically synthesized fragments. In some embodiments, the synthetic IL-7 polypeptide is prepared from 4 or 5 chemically synthesized fragments.

In some embodiments, the synthetic IL-7 polypeptide comprises a homoserine (Hse) residue located in any one of amino acid residues 31-41. In some embodiments, the synthetic IL-7 polypeptide comprises a Hse residue located in any one of amino acid residues 71-81. In some embodiments, the synthetic IL-7 polypeptide comprises a Hse residue located in any one of amino acid residues 109-119. In some embodiments, the synthetic IL-7 polypeptide comprises 1, 2, 3, or more Hse residues. In some embodiments, the synthetic IL-7 polypeptide comprises Hse36, Hse76, Hse 114, or a combination thereof. In some embodiments, the synthetic IL-7 polypeptide comprises Hse36, Hse76, and Hse114. In some embodiments, the synthetic IL-7 polypeptide comprises at least two amino acid substitutions, wherein the at least two amino acid substitutions are selected from (a) a homoserine (Hse) residue located in any one of amino acid residues 31-41; (b) a homoserine residue located in any one of amino acid residues 71-81; and (c) a homoserine residue located in any one of amino acid residues 109-119. In some embodiments, the synthetic IL-7 polypeptide comprises Hse36 and Hse76. In some embodiments, the synthetic IL-7 polypeptide comprises Hse36 and Hse114. In some embodiments, the synthetic IL-7 polypeptide comprises Hse76 and Hse114. In some embodiments, the synthetic IL-7 polypeptide comprises Hse36. In some embodiments, the synthetic IL-7 polypeptide comprises Hse76. In some embodiments, the synthetic IL-7 polypeptide comprises Hse114. In some embodiments, the synthetic IL-7 polypeptide comprises 1, 2, 3, 4, 5, or more norleucine (Nle) residues. In some embodiments, the synthetic IL-7 polypeptide comprises a Nle residue located in any one of residues 12-22. In some embodiments, the synthetic IL-7 polypeptide comprises one or more Nle residues located in any one of amino acid residues 22-32. In some embodiments, the synthetic IL-7 polypeptide comprises a Nle residue located in any one of amino acid residues 49-59. In some embodiments, the synthetic IL-7 polypeptide comprises a Nle residue located in any one of amino acid residues 64-74. In some embodiments, the synthetic IL-7 polypeptide comprises a Ne residue located in any one of amino acid residues 142-152. In some embodiments, the synthetic IL-7 polypeptide comprises five Ne substitutions. In some embodiments, the synthetic IL-7 polypeptide comprises Nle17, Nle27, Nle54, Nle69, and Nle147. In some embodiments, the synthetic IL-7 polypeptide comprises SEQ ID NO: 3.

In some embodiments, the synthetic IL-7 polypeptide comprises a homoserine (Hse) residue at one or more positions within the synthetic polypeptide. In some embodiments, the synthetic IL-7 polypeptide comprises a homoserine residue at a position selected from the region of residues 26-46, residues 66-86, and residues 104-124, wherein residue position numbering of the synthetic IL-7 polypeptide is based on SEQ ID NO: 1 as a reference sequence. In some embodiments, the synthetic IL-7 comprises homoserine residues at positions selected from the region of residues 29-42, residues 69-83, and residues 107-124 of the synthetic IL-7 polypeptide. In some embodiments, the synthetic IL-7 comprises homoserine residues at positions selected from the region of residues 31-41, residues 71-81, and residues 109-119 of the synthetic IL-7 polypeptide. In some embodiments, the synthetic IL-7 comprises homoserine residues at positions selected from the region of residues 33-39, residues 73-79, and residues 111-117 of the synthetic IL-7 polypeptide. In some embodiments, the synthetic IL-7 comprises homoserine residues at positions selected from the region of residues 34-38, residues 74-78, and residues 112-116 of the synthetic IL-7 polypeptide. In some embodiments, the synthetic IL-7 polypeptide comprises a homoserine in one, two, or three of the regions provided herein.

In some embodiments, the synthetic IL-7 polypeptide comprises a Hse residue in one or more of the regions of residues 31-41, residues 71-81, and residues 109-119, wherein residue position numbering of the synthetic IL-7 polypeptide is based on SEQ ID NO: 1 as a reference sequence. In some embodiments, the synthetic IL-7 polypeptide comprises a Hse residue in one or more of the regions of residues 31-41, residues 71-81, and residues 109-119. In some embodiments, the synthetic IL-7 polypeptide comprises a Hse residue in two of the regions of residues 31-41, residues 71-81, and residues 109-119. In some embodiments, the synthetic IL-7 polypeptide comprises a Hse residue in two of the regions of residues 31-41, residues 71-81, and residues 109-119. In some embodiments, the synthetic IL-7 polypeptide comprises a Hse residue in each the regions of residues 31-41, residues 71-81, and residues 109-119.

In some embodiments, the synthetic IL-7 polypeptide comprises a Hse residue at position 36. In some embodiments, the synthetic IL-7 polypeptide comprises a Hse residue at position 76. In some embodiments, the synthetic IL-7 polypeptide comprises a Hse residue at position 114. In some embodiments, the synthetic IL-7 polypeptide comprises Hse residues at one, two, or three of residues 36, 76, and 114. In some embodiments, the synthetic IL-7 polypeptide comprises Hse residues at one, two, or three of residues 36, 76, and 114. In some embodiments, the synthetic IL-7 polypeptide comprises Hse residues at two or more of positions 36, 76, and 114. In some embodiments, the synthetic IL-7 polypeptide comprises Hse residues at positions 36, 76, and 114.

In some embodiments, the synthetic IL-7 polypeptide comprises one or more amino acid substitutions selected from: (a) a homoserine residue located at any one of residues 31-41; (b) a homoserine residue located at any one of residues 71-81; (c) a homoserine residue located at any one of residues 109-119; (d) a norleucine or O-methyl-homoserine residue located at any one of residues 12-22; (e) a norleucine or O-methyl-homoserine residue located at any one of residues 22-32; (f) a norleucine or O-methyl-homoserine residue located at any one of residues 49-59; (g) a norleucine or O-methyl-homoserine residue located at any one of residues 64-74; and (h) a norleucine or O-methyl-homoserine residue located at any one of residues 142-152; wherein residue position numbering of the synthetic IL-7 polypeptide is based on SEQ ID NO: 1 as a reference sequence. In some embodiments, the synthetic IL-7 polypeptide comprises one homoserine at each of (a)-(c).

In some embodiments, the synthetic IL-7 polypeptide comprises one or more amino acid substitutions selected from: (a) an O-methyl-homoserine residue located at any one of residues 12-22, (b) an O-methyl-homoserine residue located at any one of residues 22-32 (c) a homoserine residue located at any one of residues 31-41; (d) an O-methyl-homoserine residue located at any one of residues 49-59, (e) an O-methyl-homoserine residue located at any one of residues 64-74. (f) a homoserine residue located at any one of residues 71-81; (g) a homoserine residue located at any one of residues 109-119; and (h) an O-methyl-homoserine residue located at any one of residues 142-152, wherein residue position numbering of the synthetic IL-7 polypeptide is based on SEQ ID NO: 1 as a reference sequence. In some embodiments, the synthetic IL-7 polypeptide comprises 1, 2, 3, 4, 5, 6, 7, or 8 of the amino acid substitutions of (a)-(h).

In some embodiments, the synthetic IL-7 polypeptide comprises one or more amino acid substitutions selected from: (a) a homoserine residue located at any one of residues 31-41; (b) a homoserine residue located at any one of residues 71-81; (c) a homoserine residue located at any one of residues 109-119; (d) a norleucine residue located at any one of residues 12-22; (e) a norleucine residue located at any one of residues 22-32; (f) a norleucine residue located at any one of residues 49-59; (g) a norleucine residue located at any one of residues 64-74; and (h) a norleucine residue located at any one of residues 142-152; wherein residue position numbering of the synthetic IL-7 polypeptide is based on SEQ ID NO: 1 as a reference sequence. In some embodiments, the synthetic IL-7 polypeptide comprises 1, 2, 3, 4, 5, 6, 7, or 8 of the amino acid substitutions of (a)-(h).

In some embodiments, the synthetic IL-7 polypeptide comprises an amino acid substitution of at least one methionine residue in SEQ ID NO: 1. In some embodiments, the amino acid substitution of at least one methionine residue comprises a substitution at M17, M27, M54, M69, or M147. In some embodiments, the synthetic IL-7 polypeptide comprises substitutions of one, two, three, or four methionine residues. In some embodiments, the synthetic IL-7 polypeptide comprises substitutions of at least two methionine residues. In some embodiments, the synthetic IL-7 polypeptide comprises substitutions of at least three methionine residues. In some embodiments, the synthetic IL-7 polypeptide comprises substitutions of at least four methionine residues. In some embodiments, the synthetic IL-7 polypeptide comprises substitutions of all five methionine residues

In some embodiments, one or more methionine residues in the synthetic IL-7 polypeptide of SEQ ID NO: 1 are substituted for residues that do not contain sulfur atoms. In some embodiments, one or more methionine residues are each independently substituted for a methionine isostere. In some embodiments, one or more methionine residues are each independently substituted for norleucine (Nle) or O-methyl-homoserine (Omh). In some embodiments, at least one methionine residue is substituted for a Nle or Omh residue. In some embodiments, one methionine residue is substituted for Nle on Omh residue. In some embodiments, two methionine residues are each independently substituted for Nle or Omh residues. In some embodiments, three methionine residues are each independently substituted for Nle or Omh residues. In some embodiments, four methionine residues are each independently substituted for Nle or Omh residues. In some embodiments, each methionine is independently substituted for a Nle or Omh residue.

In some embodiments, the synthetic IL-7 peptide comprises an amino acid substitution with norleucine. In some embodiments, the synthetic IL-7 peptide comprises an amino acid substitution with norleucine at positions Met 17, Met 27, Met 54, Met 69 or Met 147. In some embodiments, the synthetic IL-7 polypeptide comprises one or more amino acid substitutions selected from norleucine (Nle) 17, O-methyl-homoserine (Omh) 17, Nle 27, Omh 27, homoserine (Hse) 36, Nle54, Omh54, Nle69, Omh69, Hse76, Hse114, Nle147, and Omh147. In some embodiments, each methionine is substituted with Nle or Omh.

In some embodiments, at least one methionine residue is substituted for a Nle residue. In some embodiments, one methionine residue is substituted for Nle residue. In some embodiments, two methionine residues are substituted for Nle residues. In some embodiments, three methionine residues are substituted for Nle residues. In some embodiments, four methionine residues are substituted for Nle residues. In some embodiments, each methionine substitution is for Nle residues.

In some embodiments, the synthetic IL-7 peptide comprises an amino acid substitution with O-methyl-L-homoserine. In some embodiments, the synthetic IL-7 peptide comprises an amino acid substitution with O-methyl-L-homoserine at positions Met 17, Met 27, Met 54, Met 69, or Met 147. In some embodiments, the synthetic IL-7 polypeptide comprises one or more amino acid substitutions selected from norleucine (Nle) 17, O-methyl-homoserine (Omh) 17, Nle27, Omh27, homoserine (Hse) 36, Nle54, Omh54, Nle69, Omh69, Hse76, Hse114, Nle147, and Omh147.

In some embodiments, the synthetic IL-7 polypeptide comprises an amino acid sequence at least 80%, 85%, 90%, 95%, 99%, or 100% identical to SEQ ID NO: 3. In some embodiments, the synthetic IL-7 polypeptide comprises an amino acid sequence of SEQ ID NO: 3. In some embodiments, the synthetic IL-7 polypeptide consists of an amino acid sequence at least 80%, 85%, 90%, 95%, 99%, or 100% identical to the sequence of SEQ ID NO: 3.

In some embodiments, the synthetic IL-7 polypeptide comprises an amino acid sequence at least about 75% identical to that of SEQ ID NO: 3. In some embodiments, the synthetic IL-7 polypeptide comprises an amino acid sequence at least about 80% identical to that of SEQ ID NO: 3. In some embodiments, the synthetic IL-7 polypeptide comprises an amino acid sequence at least about 85% identical to that of SEQ ID NO: 3. In some embodiments, the synthetic IL-7 polypeptide comprises an amino acid sequence at least about 90% identical to that of SEQ ID NO: 3. In some embodiments, the synthetic IL-7 polypeptide comprises an amino acid sequence at least about 95% identical to that of SEQ ID NO: 3. In some embodiments, the synthetic IL-7 polypeptide comprises an amino acid sequence at least about 96% identical to that of SEQ ID NO: 3. In some embodiments, the synthetic IL-7 polypeptide comprises an amino acid sequence at least about 97% identical to that of SEQ ID NO: 3. In some embodiments, the synthetic IL-7 polypeptide comprises an amino acid sequence at least about 98% identical to that of SEQ ID NO: 3. In some embodiments, the synthetic IL-7 polypeptide comprises an amino acid sequence at least about 99% identical to that of SEQ ID NO: 3. In some embodiments, the synthetic IL-7 polypeptide comprises an amino acid sequence identical to that of SEQ ID NO: 3.

In some embodiments, the synthetic IL-7 polypeptide comprises an amino acid sequence at least 80%, 85%, 90%, 95%, 99%, or 100% identical to SEQ ID NOs: 4. In some embodiments, the synthetic IL-7 polypeptide comprises an amino acid sequence of SEQ ID NO: 4. In some embodiments, the synthetic IL-7 polypeptide consists of an amino acid sequence at least 80%, 85%, 90%, 95%, 99%, or 100% identical to the sequence of SEQ ID NO: 4.

In some embodiments, the synthetic IL-7 polypeptide comprises an amino acid sequence at least 80%, 85%, 90%, or 95% identical to SEQ ID NOs: 1. In some embodiments, the synthetic IL-7 polypeptide consists of an amino acid sequence at least 80%, 85%, 90%, or 95% identical to the sequence of SEQ ID NO: 1.

### Methods of Synthesizing IL-7 Polypeptides

Also provided herein is a method synthesizing an IL-7 polypeptide. In some cases, the IL-7 polypeptide is synthesized chemically rather than recombinantly expressed. In some instances, several fragment peptide precursors of the synthetic IL-7 polypeptide are synthesized and subsequently ligated together using a suitable ligation methodology (e.g., alpha-keto acid hydroxylamine (KAHA) ligation). In some cases, after ligation, the resulting synthetic IL-7 polypeptide is folded to produce a synthetic IL-7 polypeptide having a secondary and tertiary structure substantially identical to that of a recombinant or wild type IL-7 polypeptide

An exemplary, non-limiting synthetic scheme of an IL-7 polypeptide as provided herein is shown in FIG. 4. In general, in some embodiments, a first fragment (“Segment 1”) containing amino acids or amino acid precursors corresponding to residue numbers 1-35 of the synthetic IL-7 polypeptide is prepared (e.g., by solid phase peptide synthesis (SPPS)), as compared to the amino acid sequence set for in SEQ ID NO: 1. This is coupled to a second fragment (“Segment 2”) containing, in some embodiments, amino acids or amino precursors corresponding to residue numbers 36-75 of the synthetic IL-7 polypeptide to produce a single fragment (“Segment 12”). This second fragment is in some embodiments also prepared by SPPS. Similarly, a third fragment is prepared, in some embodiments by SPPS, having amino acids or amino acid precursors corresponding to either residue numbers 76-113 of the synthetic IL-7 polypeptide. This third fragment is coupled to a fourth fragment (“Segment 4”), in some embodiments prepared by SPPS, which contains amino acids or amino acid precursors corresponding to residue numbers 114-152 of the synthetic IL-7 polypeptide to produce a single fragment (“Segment 34”). Segment 12 and Segment 34 are then coupled to produce a full-length fragment (“Segment 1234”). In embodiments where KAHA ligation is used to ligate the fragments, the site residues are then rearranged to produce amide bonds at the ligation points (e.g., depsipeptide homoserine rearrangement to amide bond). Finally, the full-length linear fragment is then folded to produce a synthetic IL-7 polypeptide.

In one aspect, described herein, is a method of making a synthetic IL-7 polypeptide. In another aspect, described herein, is a method of making a synthetic IL-7 polypeptide comprising synthesizing two or more fragments of the synthetic IL-7 polypeptide and ligating the fragments. In another aspect, described herein, is a method of making a synthetic IL-7 polypeptide comprising a. synthesizing two or more fragments of the synthetic IL-7 polypeptide, b. ligating the fragments; and c. folding the ligated fragments. In another aspect, described herein, is a method of making a synthetic IL-7 polypeptide comprising providing two or more fragments of the synthetic IL-7 polypeptide and ligating the fragments. In another aspect, described herein, is a method of making a synthetic IL-7 polypeptide comprising a. providing two or more fragments of the synthetic IL-7 polypeptide, b. ligating the fragments; and c. folding the ligated fragments. In another aspect, described herein, is a method of making a synthetic IL-7 polypeptide comprising ligating two or more fragments of the synthetic IL-7 polypeptide, wherein at least one the two or more fragments of the synthetic IL-7 polypeptide are synthesized, and folding the ligated fragments

In some embodiments, the two or more fragments of the synthetic IL-7 polypeptide are synthesized chemically. In some embodiments, the two or more fragments of the synthetic IL-7 polypeptide are synthesized by solid phase peptide synthesis. In some embodiments, the two or more fragments of the synthetic IL-7 polypeptide are synthesized on an automated peptide synthesizer.

In some embodiments, the synthetic IL-7 polypeptide is ligated from 2, 3, 4, 5, 6, 7, 8, 9, 10, or more peptide fragments. In some embodiments, the synthetic peptide is ligated from 2 peptide fragments. In some embodiments, the synthetic IL-7 polypeptide is ligated from 3 peptide fragments. In some embodiments, the synthetic IL-7 polypeptide is ligated from 4 peptide fragments. In some embodiments, the synthetic IL-7 polypeptide is ligated from 2 to 10 peptide fragments.

In some embodiments, the two or more fragments comprise an N-terminal fragment, a C-terminal fragment, and optionally one or more interior fragments, wherein the N-terminal fragment comprises the N-terminus of the synthetic IL-7 polypeptide and the C-terminal fragment comprises the C-terminus of the synthetic IL-7 polypeptide. In some embodiments, each of the N-terminal fragment and the one or more interior fragments comprise an alpha-keto amino acid as the C-terminal residue of each fragment. In some embodiments, each alpha-keto amino acid is selected from alpha-keto-phenylalanine, alpha-keto-tyrosine, alpha-keto-valine, alpha-keto-leucine, alpha-keto-isoleucine, alpha-keto-norleucine, and alpha-keto-O-methylhomoserine.

In some embodiments, each of the C-terminal fragment and the one or more interior fragments comprise a residue having a hydroxylamine or a cyclic hydroxylamine functionality as the N-terminal residue of each fragment. In some embodiments, each residue having the hydroxylamine or the cyclic hydroxylamine functionality is a 5-oxaproline (Opr) residue.

In some embodiments, the two or more fragments of the synthetic IL-7 polypeptide are ligated together. In some embodiments, three or more fragments of the synthetic IL-7 polypeptide are ligated in a sequential fashion. In some embodiments, three or more fragments of the synthetic IL-7 polypeptide are ligated in a one-pot reaction.

In some embodiments, synthesizing two or more fragments of the synthetic IL-7 polypeptide comprises synthesizing four fragments. In some embodiments, providing two or more fragments of the synthetic IL-7 polypeptide comprises providing four fragments. In some embodiments, the four fragments include four fragments each having at least about 80% sequence identity to any sequence independently selected from those provided in SEQ ID NOs: 5-11. In some embodiments, the four fragments include four fragments having at least about 85% sequence identity to those provided in SEQ ID NOs: 5-11. In some embodiments, the four fragments include four fragments having at least about 90% sequence identity to those provided in SEQ ID NOs: 5-11. In some embodiments, the four fragments include four fragments having at least about 95% sequence identity to those provided in SEQ ID NOs: 5-11. In some embodiments, the four fragments include four fragments provided in SEQ ID NOs: 5-11.

In some embodiments, the N-terminal fragment comprises residues corresponding to residues 1-35 of SEQ ID NO: 1, the first interior fragment comprises residues corresponding to residues 36-75 of SEQ ID NO: 1, the second interior fragment comprises residues corresponding to residues 76-113 of SEQ ID NO: 1, and the C-terminal fragment comprises residues corresponding to residues 114-152 of SEQ ID NO: 1. Exemplary peptides synthesized for this strategy can be found in SEQ ID NOs: 5-11. In some embodiments, the method is used to make an IL-7 polypeptide having at least about 80%, at least about 85%, at least about 90%, or at least about 95% sequence identity to SEQ ID NO: 3.

In some embodiments, the N-terminal fragment comprises residues corresponding to residues 1-35 of SEQ ID NO: 1, the first interior fragment comprises residues corresponding to residues 36-75 of SEQ ID NO: 1, the second interior fragment comprises residues corresponding to residues 76-113 of SEQ ID NO: 1, and the C-terminal fragment comprises residues corresponding to residues 114-152 of SEQ ID NO: 1. Exemplary peptides synthesized with this strategy can be found in SEQ ID NOs: 5-11. In some embodiments, the method is used to make an IL-7 polypeptide having at least about 80%, at least about 85%, at least about 90%, or at least about 95% sequence identity to SEQ ID NOs: 3 or 4.

In some embodiments, the synthetic IL-7 is prepared from four fragments. In some embodiments, the four fragments comprise an N-terminal fragment, a first interior fragment, a second interior fragment, and a C-terminal fragment.

In some embodiments, the N-terminal fragment comprises residues which correspond to amino acids 1-35 of the synthetic IL-7 polypeptide, wherein residue position numbering of the synthetic IL-7 polypeptide is based on SEQ ID NO: 1 as a reference sequence. In some embodiments, the N-terminal fragment comprises an N-terminal extension as compared to the sequence of SEQ ID NO: 1. In some embodiments, the N-terminal fragment comprises an adduct attached to the N-terminal amine of the fragment (e.g., a conjugation handle linked to the N-terminus as provided herein). In some embodiments, the N-terminal fragment comprises an amino acid sequence having at least 80% sequence identity with the amino acid sequence as set forth in SEQ ID NO: 5. In some embodiments, the N-terminal fragment comprises an amino acid sequence having at least 85% sequence identity with the amino acid sequence as set forth in SEQ ID NO: 5. In some embodiments, the N-terminal fragment comprises an amino acid sequence having at least 95% sequence identity with the amino acid sequence as set forth in SEQ ID NO: 5. In some embodiments, the N-terminal fragment comprises an amino acid sequence identical to the amino acid sequence as set forth in SEQ ID NO: 5. In some embodiments, the N-terminal fragment comprises an amino acid sequence having at least 80% sequence identity with the amino acid sequence as set forth in SEQ ID NO: 6. In some embodiments, the N-terminal fragment comprises an amino acid sequence having at least 85% sequence identity with the amino acid sequence as set forth in SEQ ID NO: 6. In some embodiments, the N-terminal fragment comprises an amino acid sequence having at least 95% sequence identity with the amino acid sequence as set forth in SEQ ID NO: 6. In some embodiments, the N-terminal fragment comprises an amino acid sequence identical to the amino acid sequence as set forth in SEQ ID NO: 6.

In some embodiments, the first interior fragment comprises residues which correspond to amino acids 36-75 of the synthetic IL-7 polypeptide, wherein residue position numbering of the synthetic IL-7 polypeptide is based on SEQ ID NO: 1 as a reference sequence. In some embodiments, the first interior fragment comprises an amino acid sequence having at least 80% sequence identity with the amino acid sequence as set forth in SEQ ID NO: 7. In some embodiments, the first interior fragment comprises an amino acid sequence having at least 85% sequence identity with the amino acid sequence as set forth in SEQ ID NO: 7. In some embodiments, the first interior fragment comprises an amino acid sequence having at least 90% sequence identity with the amino acid sequence as set forth in SEQ ID NO: 7. In some embodiments, the first interior fragment comprises an amino acid sequence having at least 95% sequence identity with the amino acid sequence as set forth in SEQ ID NO: 7. In some embodiments, the first interior fragment comprises an amino acid sequence identical to the amino acid sequence as set forth in SEQ ID NO: 7. In some embodiments, the first interior fragment comprises an amino acid sequence having at least 80% sequence identity with the amino acid sequence as set forth in SEQ ID NO: 8. In some embodiments, the first interior fragment comprises an amino acid sequence having at least 85% sequence identity with the amino acid sequence as set forth in SEQ ID NO: 8. In some embodiments, the first interior fragment comprises an amino acid sequence having at least 90% sequence identity with the amino acid sequence as set forth in SEQ ID NO: 8. In some embodiments, the first interior fragment comprises an amino acid sequence having at least 95% sequence identity with the amino acid sequence as set forth in SEQ ID NO: 8. In some embodiments, the first interior fragment comprises an amino acid sequence identical to the amino acid sequence as set forth in SEQ ID NO: 8.

In some embodiments, the second interior fragment comprises residues which correspond to amino acids 76-113 of the synthetic IL-7 polypeptide, wherein residue position numbering of the synthetic IL-7 polypeptide is based on SEQ ID NO: 1 as a reference sequence. In some embodiments, the second interior fragment comprises an amino acid sequence having at least 80% sequence identity with the amino acid sequence as set forth in SEQ ID NO: 9. In some embodiments, the second interior fragment comprises an amino acid sequence having at least 85% sequence identity with the amino acid sequence as set forth in SEQ ID NO: 9. In some embodiments, the second interior fragment comprises an amino acid sequence having at least 90% sequence identity with the amino acid sequence as set forth in SEQ ID NO: 9. In some embodiments, the second interior fragment comprises an amino acid sequence having at least 95% sequence identity with the amino acid sequence as set forth in SEQ ID NO: 9. In some embodiments, the second interior fragment comprises an amino acid sequence identical to the amino acid sequence as set forth in SEQ ID NO: 9.

In some embodiments, the N-terminal fragment comprises residues which correspond to amino acids 114-152 of the synthetic IL-7 polypeptide, wherein residue position numbering of the synthetic IL-7 polypeptide is based on SEQ ID NO: 1 as a reference sequence. In some embodiments, the C-terminal fragment comprises an amino acid sequence having at least 80% sequence identity with the amino acid sequence as set forth in SEQ ID NO: 10. In some embodiments, the C-terminal fragment comprises an amino acid sequence having at least 85% sequence identity with the amino acid sequence as set forth in SEQ ID NO: 10. In some embodiments, the C-terminal fragment comprises an amino acid sequence having at least 95% sequence identity with the amino acid sequence as set forth in SEQ ID NO: 10. In some embodiments, the C-terminal fragment comprises an amino acid sequence identical to the amino acid sequence as set forth in SEQ ID NO: 10. In some embodiments, the C-terminal fragment comprises an amino acid sequence having at least 80% sequence identity with the amino acid sequence as set forth in SEQ ID NO: 11. In some embodiments, the C-terminal fragment comprises an amino acid sequence having at least 85% sequence identity with the amino acid sequence as set forth in SEQ ID NO: 11. In some embodiments, the C-terminal fragment comprises an amino acid sequence having at least 95% sequence identity with the amino acid sequence as set forth in SEQ ID NO: 11. In some embodiments, the C-terminal fragment comprises an amino acid sequence identical to the amino acid sequence as set forth in SEQ ID NO: 11.

In some embodiments, the N-terminal fragment, the first interior fragment, the second interior fragment, and the C-terminal fragment are arranged from the N-terminus to the C-terminus, respectively, in the synthetic IL-7 polypeptide.

In some embodiments, the method further comprises rearranging the ligated fragments. In some embodiments, rearranging the ligated fragments involves rearranging one or more depsipeptide bonds of the linear IL-7 polypeptide. In some embodiments, the one or more depsipeptide bonds are rearranged to form one or more amide bonds. In some embodiments, the depsipeptide bonds are formed as a result of the ligation of the fragments. In some embodiments, the depsipeptide bonds are between the hydroxyl moiety of a homoserine residue and an amino acid adjacent to the homoserine residue. In some embodiments, rearranging the ligated fragments occurs after each of the fragments have been ligated.

In some embodiments, ligated fragments are folded. In some embodiments, folding comprises forming one or more disulfide bonds within the synthetic IL-7 polypeptide. In some embodiments, the ligated fragments are subjected to a folding process. In some embodiments, the ligated fragments are folded using methods well known in the art.

In some embodiments, the ligated polypeptide or the folded polypeptide are further modified by attaching one or more additional moieties thereto. In some embodiments, the additional moiety is an additional polypeptide, such as an antibody. In some embodiments, the antibody is an anti-PD-1 antibody as provided herein. In some embodiments, the antibody is not an anti-PD-1 antibody (e.g., the antibody is specific for a different target). In some embodiments, the ligated polypeptide or the folded polypeptide are further modified by attachment of a polymer (e.g., PEGylation).

### Points of Attachment of Linkers to IL-7 Polypeptides

Provided herein are compositions comprising polypeptides, such as antibodies, which bind to PD-1 that are connected to IL-7 polypeptides by a chemical linker. As discussed supra, the chemical linker can be attached to the anti-PD-1 polypeptide at any of the positions provide herein. The second point of attachment of the linker is attached to an IL-7 polypeptide (including a synthetic IL-7) as provided herein.

In some embodiments, the chemical linker is attached to the IL-7 polypeptide at an amino acid residue. In some embodiments, the chemical linker is attached at an amino acid residue corresponding to any one of amino acid residues 1-152 of SEQ ID NO: 1 (e.g., any one of amino acid residues 1-152 of SEQ ID NO: 1).

In some embodiments, the linker is attached to a terminal amino acid residue of the IL-7 polypeptide. In some embodiments, the linker is attached to the N-terminal residue or the C-terminal residue of the IL-7 polypeptide. In some embodiments, the linker is attached to the N-terminal amino group of the IL-7 polypeptide or the C-terminal carboxyl group of the IL-7 polypeptide. In some embodiments, the N-terminal residue is a residue corresponding to position 1 of SEQ ID NO: 1. In some embodiments, the IL-7 polypeptide comprises a truncation of one or more amino acid residues from the N-terminus of SEQ ID NO: 1 (e.g., a deletion of 1, 2, 3, 4, 5, 6, 7, 8, 9, 10, or more amino acid residues) and the linker is attached to the residue which now comprises the N-terminus (e.g., for a truncation of one amino acid, the linker is attached to a residue at a position corresponding to residue 2 of SEQ ID NO: 1). In some embodiments, the IL-7 polypeptide comprises a truncation of one or more amino acid residues from the C-terminus of SEQ ID NO: 1 (e.g., a deletion of 1, 2, 3, 4, 5, 6, 7, 8, 9, 10, or more amino acid residues) and the linker is attached to the residue which now comprises the C-terminus (e.g., for a truncation of one amino acid, the linker is attached to a residue at a position corresponding to residue 151 of SEQ ID NO: 1).

In some embodiments, the linker is attached to the N-terminal amino acid residue of the IL-7 polypeptide. In some embodiments, the linker is attached to the N-terminal amino group of the IL-7 polypeptide. In some embodiments, the linker is attached to the N-terminal amino group of the IL-7 polypeptide through by a reaction with an adduct attached to the N-terminal amino group having a structure

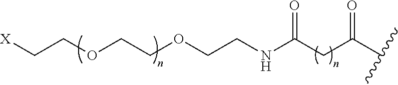

wherein each n is independently an integer from 1-30 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, or 30), and wherein X is a conjugation handle (e.g., an azide or other conjugation handle provided herein, such as a DBCO group). In some embodiments, the adduct has the structure

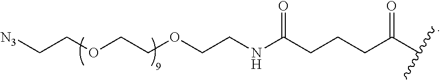

In some embodiments, the IL-7 polypeptide comprises a conjugation handle attached to one or more residues to facilitate attachment of the linker to the polypeptide which selectively binds to PD-1. The conjugation handle may be any such conjugation handle provided herein and may be attached at any residue to which the linker may be attached. In some embodiments, the conjugation handle is attached to the N-terminal residue of the polypeptide. In some embodiments, the conjugation handle comprises an azide or an alkyne.

### Biological Activity

In some embodiments, an IL-7 polypeptide described herein is capable of expanding CD4+ helper cell, CD8+ central memory cell, CD8+ effector memory cell, naïve CD8+ cell, Natural Killer (NK) cell, Natural killer T (NKT) cell populations, or a combination thereof. In some embodiments, a synthetic IL-7 polypeptide as described herein is capable of expanding CD4+ helper cell, CD8+ central memory cell, CD8+ effector memory cell, naïve CD8+ cell, Natural Killer (NK) cell, Natural killer T (NKT) cell populations, or a combination thereof. In some embodiments, an IL-7 polypeptide described herein is capable of inducing STAT5 phosphorylation in a CD8 naïve cell, a CD4 naïve cell, a CD8 memory cell, a CD4 memory cell, or a CD4 Treg cell, or any combination thereof. In some embodiments, a synthetic IL-7 polypeptide as provided herein is capable of activating one or more T-cell subtypes in a manner similar or substantially identical to a recombinant or wild type IL-7 polypeptide (e.g., exhibits an EC50 of no more than 100-fold greater than, or an EC50 of no more than 10-fold greater than a corresponding recombinant IL-7 polypeptide).

In some embodiments, the synthetic IL-7 polypeptide exhibits a half maximal effective concentration (EC50) for inducing STAT5 phosphorylation in at least one T-cell subtype which is comparable to a corresponding wild type or recombinant IL-7. In some embodiments, the EC50 of the synthetic IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater than, 20-fold greater than, 50-fold greater than, or 100-fold greater than that of a corresponding recombinant IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the polypeptide which binds specifically to PD-1 exhibits a half maximal effective concentration (EC50) for inducing STAT5 phosphorylation in at least one T-cell subtype which is comparable to wild type IL-7 when attached to the polypeptide which binds specifically to PD-1. In some embodiments, the EC50 of the IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater than, 20-fold greater than, 50-fold greater than, or 100-fold greater than that of wild type IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell.

In some embodiments, the IL-7 polypeptide conjugated to the polypeptide which binds specifically to PD-1 exhibits a half maximal effective concentration (EC50) for inducing STAT5 phosphorylation in at least one T-cell subtype which is the unconjugated IL-7 polypeptide (e.g., attaching the IL-7 polypeptide to the polypeptide which binds specifically to PD-1 does not substantially diminish the activity of the IL-7 polypeptide). In some embodiments, the EC50 of the IL-7 for inducing STAT5 phosphorylation in the at least one T-cell subtype is no more than 2-fold greater than, 3-fold greater than, 4-fold greater than, 5-fold greater than, 6-fold greater than, 7-fold greater than, 8-fold greater than, 9-fold greater than, 10-fold greater, 20-fold greater than, 50-fold greater than, or 100-fold greater than that the unconjugated IL-7. In some embodiments, the T-cell subtype is a CD8 naïve cell, a CD4 naïve cell, a CD8 memory cell, a CD4 memory cell, or a CD4 Treg cell. In some embodiments, the T-cell subtype is each of a CD8 naïve cell, a CD4 naïve cell, a CD8 memory cell, a CD4 memory cell, and a CD4 Treg cell. In some embodiments, an immunoconjugate composition provided herein (e.g., a polypeptide which binds to PD-1 (e.g., an anti-PD-1 antibody) attached to an IL-7 polypeptide through a linker, such as a chemical linker) maintains binding affinity associated with at least one of the components after formation of the linkage between the two groups. For example, in an immunoconjugate composition comprising an anti-PD-1 antibody or antigen binding fragment linked to an IL-7 polypeptide, in some embodiments the anti-PD-1 antibody or antigen binding fragment thereof retains binding to one or more Fc receptors. In some embodiments, the composition displays binding to one or more Fc receptors which is reduced by no more than about 5-fold, no more than about 10-fold, no more than about 15-fold, or no more than about 20-fold compared to the unconjugated antibody. In some embodiments, the one or more Fc receptors is the FcRn receptor, CD16a, the FcγRI receptor (CD64), the FcγRIIa receptor (CD32a), the FcγRIIβ receptor (CD32β), or any combination thereof. In some embodiments, binding of the composition to each of the FcRn receptor, CD16a, the FcγRI receptor (CD64), the FcγRIIa receptor (CD32a), and the FcγRIIβ receptor (CD32β) is reduced by no more than about 10-fold compared to the unconjugated antibody.

In some embodiments, binding of the polypeptide which binds to PD-1 (e.g., the antibody) is substantially unaffected by the conjugation with the IL-7 polypeptide. In some embodiments, the binding of the polypeptide to PD-1 is reduced by no more than about 5% compared to the unconjugated antibody. In some embodiments, the binding of the polypeptide to PD-1 is reduced by no more than about 2-fold, 3-fold, 4-fold, 5-fold, 6-fold, 7-fold, 8-fold, 9-fold, 10-fold, 20-fold, 50-fold, or 100-fold compared to the unconjugated antibody.

### Polymers

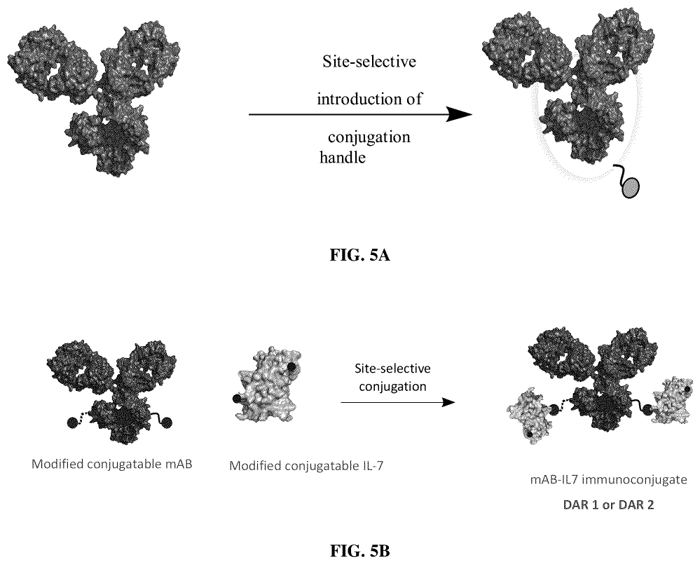

In some embodiments, a herein described the IL-7 polypeptide comprises a polymer covalently attached thereon. An exemplary 3D representation of an IL-7 polypeptide with a polymer attached to the N-terminal residue is shown in FIG. 5C. In some embodiments, the described IL-7 polypeptide comprises one or more polymers covalently attached to the IL-7 polypeptide. In some embodiments, the described IL-7 polypeptide comprises a polymer. In some embodiments, the polymer comprises at least a portion of the linker which attached the IL-7 polypeptide to the polypeptide which selectively binds to PD-1.

In some embodiments, the polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide). In some embodiments, the water-soluble polymer is polysaccharide. In some embodiments, the water-soluble polymer is poly(ethylene oxide).

In some embodiments, a IL-7 polypeptide described herein comprises a polymer covalently attached to the N-terminus of the IL-7 polypeptide. In some embodiments, the polymer comprises at least a portion of the linker used to attach the IL-7 polypeptide to the polypeptide which selectively binds to PD-1.

In some embodiments, the attached polymer has a weight average molecular weight of about 120 Daltons to about 1,000 Daltons. In some embodiments, the polymer has a weight average molecular weight of about 120 Daltons to about 250 Daltons, about 120 Daltons to about 300 Daltons, about 120 Daltons to about 400 Daltons, about 120 Daltons to about 500 Daltons, about 120 Daltons to about 1,000 Daltons, about 250 Daltons to about 300 Daltons, about 250 Daltons to about 400 Daltons, about 250 Daltons to about 500 Daltons, about 250 Daltons to about 1,000 Daltons, about 300 Daltons to about 400 Daltons, about 300 Daltons to about 500 Daltons, about 300 Daltons to about 1,000 Daltons, about 400 Daltons to about 500 Daltons, about 400 Daltons to about 1,000 Daltons, or about 500 Daltons to about 1,000 Daltons. In some embodiments, the polymer has a weight average molecular weight of about 120 Daltons, about 250 Daltons, about 300 Daltons, about 400 Daltons, about 500 Daltons, or about 1,000 Daltons. In some embodiments, the polymer has a weight average molecular weight of at least about 120 Daltons, about 250 Daltons, about 300 Daltons, about 400 Daltons, or about 500 Daltons. In some embodiments, the polymer has a weight average molecular weight of at most about 250 Daltons, about 300 Daltons, about 400 Daltons, about 500 Daltons, or about 1,000 Daltons. In some embodiments, the polymer has a weight average molecular weight of about 10 kDa to about 50 kDa. In some embodiments, the polymer has a weight average molecular weight of about 10 kDa, about 20 kDa, or about 30 kDa. In some embodiments, the polymer has a weight average molecular weight of about 30 kDa.

In some embodiments, the attached polymer comprises a water-soluble polymer. In some embodiments, the water-soluble polymer comprises poly(alkylene oxide), polysaccharide, poly(vinyl pyrrolidone), poly(vinyl alcohol), polyoxazoline, poly(acryloylmorpholine), or a combination thereof. In some embodiments, the water-soluble polymer is poly(alkylene oxide) such as polyethylene glycol (e.g., polyethylene oxide). In some embodiments, the water-soluble polymer is polyethylene glycol. In some embodiments, the water-soluble polymer comprises modified poly(alkylene oxide). In some embodiments, the modified poly(alkylene oxide) comprises one or more linker groups. In some embodiments, the one or more linker groups comprise bifunctional linkers such as an amide group, an ester group, an ether group, a thioether group, a carbonyl group and alike. In some embodiments, the one or more linker groups comprise an amide linker group. In some embodiments, the modified poly(alkylene oxide) comprises one or more spacer groups. In some embodiments, the spacer groups comprise a substituted or unsubstituted C₁-C₆ alkylene group. In some embodiments, the spacer groups comprise —CH₂—, —CH₂CH₂—, or —CH₂CH₂CH₂—. In some embodiments, the linker group is the product of a biorthogonal reaction (e.g., biocompatible and selective reactions). In some embodiments, the bioorthogonal reaction is a Cu(I)-catalyzed or “copper-free” alkyne-azide triazole-forming reaction, the Staudinger ligation, inverse-electron-demand Diels-Alder (IEDDA) reaction, “photo-click” chemistry, or a metal-mediated process such as olefin metathesis and Suzuki-Miyaura or Sonogashira cross-coupling. In some embodiments, the first polymer comprises at least a portion of the linker which attaches the IL-7 polypeptide to the polypeptide which selectively binds to PD-1.

In some embodiments, an IL-7 polypeptide provided herein comprises a reaction group that facilitates the conjugation of the IL-7 polypeptide with a derivatized molecule or moiety such as an antibody and a polymer. An exemplary 3D representation of an IL-7 polypeptide with a reaction group for the attachment of a polymer (or another moiety, such as an antibody as provided herein) is shown in FIG. 5B. In some embodiments, the reaction group comprises one or more of: carboxylic acid derived active esters, mixed anhydrides, acyl halides, acyl azides, alkyl halides, N-maleimides, imino esters, isocyanates, and isothiocyanates. In some embodiments, the reaction group comprises azides. In some embodiments, the reaction group forms a part of the linker which attaches the IL-7 polypeptide to the polypeptide which selectively binds to PD-1.

In some embodiments, the water-soluble polymer comprises from 1 to 10 polyethylene glycol chains. I

In some embodiments, each of the polyethylene glycol chains is independently terminally capped with a hydroxy, an alkyl, an alkoxy, an amido, or an amino group. In some embodiments, each of the polyethylene glycol chains is independently terminally capped with an amino group. In some embodiments, each of the polyethylene glycol chains is independently terminally capped with an amido group. In some embodiments, each of the polyethylene glycol chains is independently terminally capped with an alkoxy group. In some embodiments, each of the polyethylene glycol chains is independently terminally capped with an alkyl group. In some embodiments, each of the polyethylene glycol chains is independently terminally capped with a hydroxy group.

In some embodiments, one or more of the covalently attached polymers comprise a linker. In some embodiments, one or more of the covalently attached polymers, such as the third polymer, comprises one or more linkers. In some embodiments, the linker comprises one or more amino acids. In some embodiments, the linker comprises one or more lysines. In some embodiments, the linker comprises a spacer. In some embodiments, the linker comprises reactive functional groups or functional groups such as amide.

In some embodiments, the water-soluble polymer attached at the amino terminal residue of IL-7 comprises one or more linkers and/or spacers. In some embodiments, the water-soluble polymer attached at the amino terminal residue comprises a point of attachment to the polypeptide which selectively binds to PD-1. In some embodiments, the one or more linkers comprise one or more amide groups.

In some embodiments, the polymers are synthesized from suitable precursor materials. In some embodiments, the polymers are synthesized from the precursor materials of, Structure 6, Structure 7, Structure 8, or Structure 9, wherein Structure 6 is:

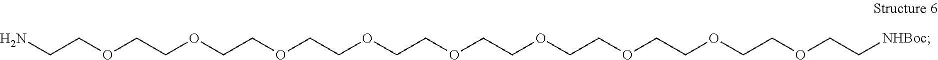 [SMILES: CC(C)(C)OC(=O)NCCOCCOCCOCCOCCOCCOCCOCCOCCOCCN]

### Structure 7 is:

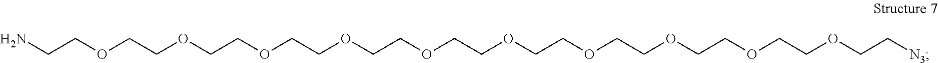 [SMILES: [N-]=[N+]=NCCOCCOCCOCCOCCOCCOCCOCCOCCOCCOCCN]

### Structure 8 is:

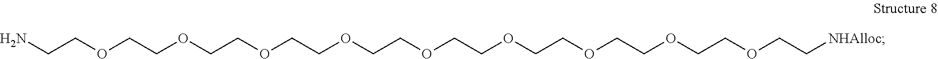

### and Structure 9 is:

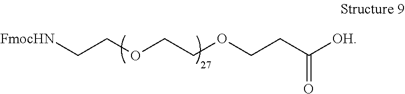

### Orthogonal Payloads

The anti-PD-1-IL-7 immunoconjugates of the disclosure can comprise dual orthogonal payloads. In one non-limiting instance, the anti-PD-1-IL-7 immunoconjugates can comprise an anti-PD-1 polypeptide, one modified IL-7 polypeptide, and one payload that linked to the anti-PD-1 polypeptide by a chemical orthogonal linking group. The orthogonal payload can be an amino acid, amino acid derivative, peptide, protein, cytokine, alkyl group, aryl or heteroaryl group, therapeutic small molecule drug, polyethylene glycol (PEG) moiety, lipid, sugar, biotin, biotin derivative, deoxyribonucleic acid (DNA), ribonucleic acid (RNA), or peptide nucleic acid (PNA), any of which is substituted, unsubstituted, modified, or unmodified. In some embodiments, the orthogonal payload is a therapeutic small molecule. In some embodiments, the orthogonal payload is a PEG moiety. In some embodiments, the orthogonal payload is an additional cytokine, for example, IL-2 or IL-18. In one exemplary instance, human IL-2 has an amino acid sequence of APTSSSTKKTQLQLEHLLLDLQMILNGINNYKNPKLTRMLTFKFYMPKKATELKHLQCL EEELKPLEEVLNLAQSKNFHLRPRDLISNINVIVLELKGSETTFMCEYADETATIVEFLNR WITFCQSIISTLT (SEQ ID NO: 117), or is a modified IL-2. In one exemplary instance, human IL-18 has an amino acid sequence of YFIAEDDENLESDYFGKLESKLSVIRNLNDQVLFIDQGNRPLFEDMTDSDCRDNAPRTIFI ISMYKDSQPRGMAVTISVKCEKISTLSCENKIISFKEMNPPDNIKDTKSDIIFFQRSVPGHD NKMQFESSSYEGYFLACEKERDLFKLILKKEDELGDRSIIVFTVQNED (SEQ ID NO: 118), or is a modified IL-18. In some instances, a conjugation handle can be added at one or more of Cys68, Glu69, Lys70 of IL-18.

### Pharmaceutical Formulations

In one aspect, described herein is a pharmaceutical formulation comprising: a polypeptide which selectively binds to PD-1 linked to an IL-7 polypeptide described herein; and a pharmaceutically acceptable carrier or excipient. In some embodiments, the pharmaceutical formulation further comprises one or more excipients, wherein the one or more excipients include, but are not limited to, a carbohydrate, an inorganic salt, an antioxidant, a surfactant, a buffer, or any combination thereof. In some embodiments the pharmaceutical formulation further comprises one, two, three, four, five, six, seven, eight, nine, ten, or more excipients, wherein the one or more excipients include, but are not limited to, a carbohydrate, an inorganic salt, an antioxidant, a surfactant, a buffer, or any combination thereof.

In some embodiments, the pharmaceutical formulation further comprises a carbohydrate. In certain embodiments, the carbohydrate is selected from the group consisting of fructose, maltose, galactose, glucose, D-mannose, sorbose, lactose, sucrose, trehalose, cellobiose raffinose, melezitose, maltodextrins, dextrans, starches, mannitol, xylitol, maltitol, lactitol, xylitol, sorbitol (glucitol), pyranosyl sorbitol, myoinositol, cyclodextrins, and combinations thereof.

Alternately, or in addition, the pharmaceutical formulation further comprises an inorganic salt. In certain embodiments, the inorganic salt is selected from the group consisting of sodium chloride, potassium chloride, magnesium chloride, calcium chloride, sodium phosphate, potassium phosphate, sodium sulfate, or combinations thereof.

Alternately, or in addition, the pharmaceutical formulation Composition C comprises an antioxidant. In certain embodiments, the antioxidant is selected from the group consisting of ascorbyl palmitate, butylated hydroxyanisole, butylated hydroxytoluene, potassium metabisulfite, propyl gallate, sodium metabisulfite, sodium thiosulfate, vitamin E, 3,4-dihydroxybenzoic acid, and combinations thereof.

Alternately, or in addition, the pharmaceutical formulation further comprises a surfactant. In certain embodiments, the surfactant is selected from the group consisting of polysorbates, sorbitan esters, lipids, phospholipids, phosphatidylethanolamines, fatty acids, fatty acid esters, steroids, EDTA, zinc, and combinations thereof.

Alternately, or in addition, the pharmaceutical formulation further comprises a buffer. In certain embodiments, the buffer is selected from the group consisting of citric acid, sodium phosphate, potassium phosphate, acetic acid, ethanolamine, histidine, amino acids, tartaric acid, succinic acid, fumaric acid, lactic acid, tris, HEPES, or combinations thereof.

In some embodiments, the pharmaceutical formulation is formulated for parenteral or enteral administration. In some embodiments, the pharmaceutical composition is formulated for intravenous (IV) or subcutaneous (SQ) administration. In some embodiments, the pharmaceutical formulation is in a lyophilized form.

In one aspect, described herein is a liquid or lyophilized composition that comprises a described a polypeptide which selectively binds to PD-1 linked to an IL-7 polypeptide. In some embodiments, the polypeptide which selectively binds to PD-1 linked to the IL-7 polypeptide modified is a lyophilized powder. In some embodiments, the lyophilized powder is resuspended in a buffer solution. In some embodiments, the buffer solution comprises a buffer, a sugar, a salt, a surfactant, or any combination thereof. In some embodiments, the buffer solution comprises a phosphate salt. In some embodiments, the phosphate salt is sodium Na₂HPO₄. In some embodiments, the salt is sodium chloride. In some embodiments, the buffer solution comprises phosphate buffered saline. In some embodiments, the buffer solution comprises mannitol. In some embodiments, the lyophilized powder is suspended in a solution comprising about 10 mM Na₂HPO₄ buffer, about 0.022% SDS, and about 50 mg/mL mannitol, and having a pH of about 7.5.

### Dosage Forms

The polypeptide which selectively binds to PD-1 linked to the IL-7 polypeptides described herein can be in a variety of dosage forms. In some embodiments, polypeptide which selectively binds to PD-1 linked to the IL-7 polypeptide is dosed as rehydrated from a lyophilized powder. In some embodiments, the polypeptide which selectively binds to PD-1 linked to the IL-7 polypeptide is dosed as a suspension. In some embodiments, the polypeptide which selectively binds to PD-1 linked to the IL-7 polypeptide is dosed as a solution. In some embodiments, the polypeptide which selectively binds to PD-1 linked to the IL-7 polypeptide is dosed as an injectable solution. In some embodiments, the polypeptide which selectively binds to PD-1 linked to the IL-7 polypeptides is dosed as an IV solution.

### Methods of Treatment

In one aspect, described herein, is a method of treating cancer in a subject in need thereof, comprising: administering to the subject an effective amount of a polypeptide which selectively binds to PD-1 linked to an IL-7 polypeptide or a pharmaceutical composition as described herein. In some embodiments, the cancer is a solid cancer. A cancer or tumor can be, for example, a primary cancer or tumor or a metastatic cancer or tumor. Cancers and tumors to be treated include, but are not limited to, a melanoma, a lung cancer (e.g., a non-small cell lung cancer (NSCLC), a small cell lung cancer (SCLC), etc.), a carcinoma (e.g., a cutaneous squamous cell carcinoma (CSCC), a urothelial carcinoma (UC), a renal cell carcinoma (RCC), a hepatocellular carcinoma (HCC), a head and neck squamous cell carcinoma (HNSCC), an esophageal squamous cell carcinoma (ESCC), a gastroesophageal junction (GEJ) carcinoma, an endometrial carcinoma (EC), a Merkel cell carcinoma (MCC), etc.), a bladder cancer (BC), a microsatellite instability high (MSI-H)/mismatch repair-deficient (dMMR) solid tumor (e.g., a colorectal cancer (CRC)), a tumor mutation burden high (TMB-H) solid tumor, a triple-negative breast cancer (TNBC), a gastric cancer (GC), a cervical cancer (CC), a pleural mesothelioma (PM), classical Hodgkin's lymphoma (cHL), or a primary mediastinal large B cell lymphoma (PMBCL).

Combination therapies with one or more additional active agents are contemplated herein. In some embodiments, the second therapeutic agent is selected based on tumor type, tumor tissue of origin, tumor stage, or mutations in genes expressed by the tumor. For example, an anti-PD-1 antibody can be administered in combination with one or more of the following: a chemotherapeutic agent, a checkpoint inhibitor, a biologic cancer agent, a cancer-specific agent, a cytokine therapy, an anti-angiogenic drug, a drug that targets cancer metabolism, an antibody that marks a cancer cell surface for destruction, an antibody-drug conjugate, a cell therapy, a commonly used anti-neoplastic agent, a CAR-T therapy, an oncolytic virus, a non-drug therapy, a neurotransmission blocker, or a neuronal growth factor blocker.

An effective response is achieved when the subject experiences partial or total alleviation or reduction of signs or symptoms of illness, and specifically includes, without limitation, prolongation of survival. The expected progression-free survival times may be measured in months to years, depending on prognostic factors including the number of relapses, stage of disease, and other factors. Prolonging survival includes without limitation times of at least 1 month (mo), about at least 2 mos., about at least 3 mos., about at least 4 mos., about at least 6 mos., about at least 1 year, about at least 2 years, about at least 3 years, about at least 4 years, about at least 5 years, etc. Overall or progression-free survival can be also measured in months to years. Alternatively, an effective response may be that a subject's symptoms or cancer burden remain static and do not worsen. Further indications of treatment of indications are described in more detail below. In some instances, a cancer or tumor is reduced by at least 2%, 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the polypeptide which selectively binds to PD-1 linked to the IL-7 polypeptide is administered in a single dose of the effective amount of the IL-7 polypeptide, including further embodiments in which (i) the polypeptide which selectively binds to PD-1 linked to the IL-7 polypeptide is administered once a day; or (ii) the polypeptide which selectively binds to PD-1 linked to the IL-7 polypeptide is administered to the subject multiple times over the span of one day. In some embodiments, the polypeptide which selectively binds to PD-1 linked to the IL-7 polypeptide is administered daily, every other day, 3 times a week, once a week, every 2 weeks, every 3 weeks, every 4 weeks, every 5 weeks, every 3 days, every 4 days, every 5 days, every 6 days, bi-weekly, 3 times a week, 4 times a week, 5 times a week, 6 times a week, once a month, twice a month, 3 times a month, once every 2 months, once every 3 months, once every 4 months, once every 5 months, or once every 6 months. Administration includes, but is not limited to, injection by any suitable route (e.g., parenteral, enteral, intravenous, subcutaneous, etc.). Methods of Manufacturing anti-PD-1 polypeptide conjugated to IL-7

In one aspect, described herein, is a method of making a composition, comprising providing a polypeptide which selectively binds to PD-1, wherein the polypeptide which selectively binds to PD-1 comprises a reactive group (e.g., a conjugation handle), contacting the reactive group with a complementary reactive group attached to a cytokine, and forming the composition. The resulting composition is any of the compositions provided herein.

In some embodiments, the polypeptide which selectively binds to PD-1 is an antibody or an antigen binding fragment thereof. In some embodiments, providing the antibody comprising the reactive group comprises attaching the reactive group to the antibody. In some embodiments, the reactive group is added site-specifically. In some embodiments, attaching the reactive group to the antibody comprises contacting the antibody with an affinity group comprising a reactive functionality which forms a bond with a specific residue of the antibody. In some embodiments, attaching the reactive group to the antibody comprises contacting the antibody with an enzyme. In some embodiments, the enzyme is configured to site-specifically attach the reactive group to a specific residue of the antibody. In some embodiments, the enzyme is glycosylation enzyme or a transglutaminase enzyme.

In some embodiments, the method further comprises attaching the complementary reactive group to the cytokine. In some embodiments, attaching the complementary reactive group to the cytokine comprises chemically synthesizing the cytokine.

In some embodiments, the method comprises making a synthetic IL-7 polypeptide. In some embodiments, the method of making a synthetic IL-7 polypeptide comprises synthesizing two or more fragments of the synthetic IL-7 polypeptide and ligating the fragments. In some embodiments, the method of making the IL-7 polypeptide comprises a. synthesizing two or more fragments of the synthetic IL-7 polypeptide, b. ligating the fragments; and c. folding the ligated fragments.

In some embodiments, the two or more fragments of the synthetic IL-7 polypeptide are synthesized chemically. In some embodiments, the two or more fragments of the synthetic IL-7 polypeptide are synthesized by solid phase peptide synthesis. In some embodiments, the two or more fragments of the synthetic IL-7 polypeptide are synthesized on an automated peptide synthesizer.

In some embodiments, the synthetic IL-7 polypeptide is ligated from 2, 3, 4, 5, 6, 7, 8, 9, 10, or more peptide fragments. In some embodiments, the modified peptide is ligated from 2 peptide fragments. In some embodiments, the synthetic IL-7 polypeptide is ligated from 3 peptide fragments. In some embodiments, the synthetic IL-7 polypeptide is ligated from 4 peptide fragments. In some embodiments, the synthetic IL-7 polypeptide is ligated from 2 to 10 peptide fragments.

In some embodiments, the two or more fragments of the synthetic IL-7 polypeptide are ligated together. In some embodiments, three or more fragments of the synthetic IL-7 polypeptide are ligated in a sequential fashion. In some embodiments, three or more fragments of the synthetic IL-7 polypeptide are ligated in a one-pot reaction.

In some embodiments, ligated fragments are folded. In some embodiments, folding comprises forming one or more disulfide bonds within the synthetic IL-7 polypeptide. In some embodiments, the ligated fragments are subjected to a folding process. In some embodiments, the ligated fragments are folding using methods well known in the art. In some embodiments, the ligated polypeptide or the folded polypeptide are further modified by attaching one or more polymers thereto.

### Sequences (SEQ ID NOS) of IL-7 Polypeptides

TABLE 2

| SEQ ID | Sequence | |
|---|---|---|
| NO: | modifications* | Sequence |
| 1 | Native IL-7 | DCDIEGKDGK QYESVLMVSI DQLLDSMKEI |
| | | GSNCLNNEFN FFKRHICDAN KEGMFLFRAA |
| | | RKLRQFLKMN STGDFDLHLL KVSEGTTILL |
| | | NCTGQVKGRK PAALGEAQPT KSLEENKSLK |
| | | EQKKLNDLCF LKRLLQEIKT CWNKILMGTK |
| | | EH |
| 2 | Native IL-7 | DCDIEGKDGK QYESVLMVSI DQLLDSMKEI |
| | His-tag | GSNCLNNEFN FFKRHICDAN KEGMFLFRAA |
| | | RKLRQFLKMN STGDFDLHLL KVSEGTTILL |
| | | NCTGQVKGRK PAALGEAQPT KSLEENKSLK |
| | | EQKKLNDLCF LKRLLQEIKT CWNKILMGTK |
| | | EHVDHHHHHH |
| 3 | Synthetic IL-7 | DCDIEGKDGK QYESVLXVSI DQLLDSXKEI |
| | M17Nle, M27Nle, | GSNCLZNEFN FFKRHICDAN KEGXFLFRAA |
| | N36Hse, M54Nle, | RKLRQFLKXN STGDFZLHLL KVSEGTTILL |
| | M69Nle, D76Hse, | NCTGQVKGRK PAALGEAQPT KSLZENKSLK |
| | E114Hse, M147Nle | EQKKLNDLCF LKRLLQEIKT CWNKILXGTK |
| | | EH |
| | | X = Nle, Z = Hse |
| 4 | Synthetic IL-7 | DCDIEGKDGK QYESVLMVSI DQLLDSMKEI |
| | N36Hse, D76Hse, | GSNCLZNEFN FFKRHICDAN KEGMFLFRAA |
| | E114Hse | RKLRQFLKMN STGDFZLHLL KVSEGTTILL |
| | | NCTGQVKGRK PAALGEAQPT KSLZENKSLK |
| | | EQKKLNDLCF LKRLLQEIKT CWNKILMGTK |
| | | EH |
| 5 | 1-35 (N-terminal | DCDIEGKDGK QYESVLXVSI DQLLDSXKEI |
| | fragment) | GSNC(Akl) |
| | M17Nle, M27Nle | |
| 6 | 1-35 (N-terminal | DCDIEGKDGK QYESVLMVSI DQLLDSMKEI |
| | fragment) | GSNC(Akl) |
| 7 | 36-75 (First interior | (Opr)NEFN FFKRHICDAN KEGXFLFRAA |
| | fragment) | RKLRQFLKXN STGD(Akf) |
| | M54Nle, M69Nle | |
| 8 | 36-75 (First interior | (Opr)NEFN FFKRHICDAN KEGMFLFRAA |
| | fragment) | RKLRQFLKMN STGD(Akf) |
| 9 | 76-113 (Second interior | (Opr)LHLL KVSEGTTILL NCTGQVKGRK |
| | fragment) | PAALGEAQPT KS(Akl) |
| 10 | C-terminal fragment | (Opr)ENKSLK EQKKLNDLCF LKRLLQEIKT |
| | (114-152) | CWNKILXGTK EH |
| | M147Nle | |
| 11 | C-terminal fragment | (Opr)ENKSLK EQKKLNDLCF LKRLLQEIKT |
| | (114-152) | CWNKILMGTK EH |
| 12 | 1-75 (N-terminal/First | DCDIEGKDGK QYESVLXVSI DQLLDSXKEI |
| | Interior Fragment) | GSNCL(Hse)NEFN FFKRHICDAN KEGXFLFRAA |
| | | RKLRQFLKXN STGD(Akf) |
| 13 | 76-162 (Second | (Opr)LHLL KVSEGTTILL NCTGQVKGRK |
| | interior/C-terminal | PAALGEAQPT KSL(Hse)ENKSLK |
| | fragment) | EQKKLNDLCF LKRLLQEIKT CWNKILXGTK |
| | | EH |

In Table 2 above, X is NMe (norleucine), Z is Hse (homoserine), Opr is 5-oxaproline, Akf is alpha-keto-phenylalanine, Akl is alpha-keto-leucine

Table 3 below shows identification of molecules used in the Examples below

TABLE 3

| | | | | | | Antibody |
|---|---|---|---|---|---|---|
| | | VH | VL | IL-7 polypeptide | | Conjugation site |
| Composition | Antibody | Seq ID | Seq ID | Seq ID | DAR | (Eu numbering) |
| A | Pembrolizumab | 46 | 47 | 3 (Conjugated from | 1 | K248 |
| | | | | Composition AA) | | |
| B | Pembrolizumab | 46 | 47 | 3 (Conjugated from | 2 | K248 |
| | | | | Composition AA) | | |
| C | LZM-009 | 76 | 77 | 3 (Conjugated from | 1 | K248 |
| | | | | Composition AA) | | |

Although the present disclosure and its advantages have been described in detail, it should be understood that various changes, substitutions and alterations can be made herein without departing from the spirit and scope of the disclosure as defined in the appended claims.

The present disclosure is further illustrated in the following Examples which are given for illustration purposes only and are not intended to limit the disclosure in any way.

### EXAMPLES

### Example 1—General Methods of IL-7 Synthesis

General strategy: Synthetic IL-7 polypeptides are synthesized by ligating individual peptide segments prepared by solid phase peptide synthesis (SPPS). FIG. 2 shows the general synthesis scheme used to produce IL-7 linear proteins. Briefly, peptide segments (Seg1, Seg2, Seg3 and Seg4) were prepared using SPPS, and any desired modification to the amino acid sequence of wild-type IL-7 (SEQ ID NO:1) was incorporated during the synthesis. After purification of the individual fragments, IL-7-Seg1 and IL-7-Seg2 were ligated together, as well as IL-7-Seg3 and IL-7-Seg4. The resulting IL-7-Seg12 and IL-7-Seg34 were purified and ligated together to afford IL-7-Seg1234 with cysteines protected with Acm groups (IL-7-Seg1234-Acm). The Acm groups of IL-7-Seg1234-Acm were then universally deprotected and purified to afford synthetic IL-7 linear protein. The resulting synthetic IL-7 linear proteins were then rearranged and folded. Individual peptides are synthesized on an automated peptide synthesizer using the methods described below.

Materials and solvents: Fmoc-amino acids with suitable side chain protecting groups for Fmoc-SPPS, resins polyethylene glycol derivatives used for peptide functionalization and reagents were commercially available and were used without further purification. HPLC grade CH₃CN from was used for analytical and preparative RP-HPLC purification.

Loading ofprotected ketoacid derivatives (segment 1-3) on amine-based resin: 5 g of Rink-amide MBHA or ChemMatrix resin (1.8 mmol scale) was swollen in DMF for 30 min. Fmoc-deprotection was performed by treating the resin twice with 20% piperidine in DMF (v/v) at r.t. for 10 min. followed by several washes with DMF. Fmoc-AA-protected-α-ketoacid (1.8 mmol, 1.00 equiv.) was dissolved in 20 mL DMF and pre-activated with HATU (650 mg, 1.71 mmol, 0.95 equiv.) and DIPEA (396 μL, 3.6 mmol, 2.00 equiv.). The reaction mixture was added to the swollen resin. It was let to react for 6 h at r.t. under gentle agitation. The resin was rinsed thoroughly with DMF. Capping of unreacted amines on the resin was performed by addition of a solution of acetic anhydride (1.17 mL) and DIPEA (2.34 mL) in DMF (20 mL). It was let to react at r.t. for 15 min under gentle agitation. The resin was rinsed thoroughly with DCM followed by diethyl ether and dried. The loading of the resin was measured (0.25 mmol/g) following the method described in M. Gude, et al. (2003) Lett. Pept. Sci., 9, 203.

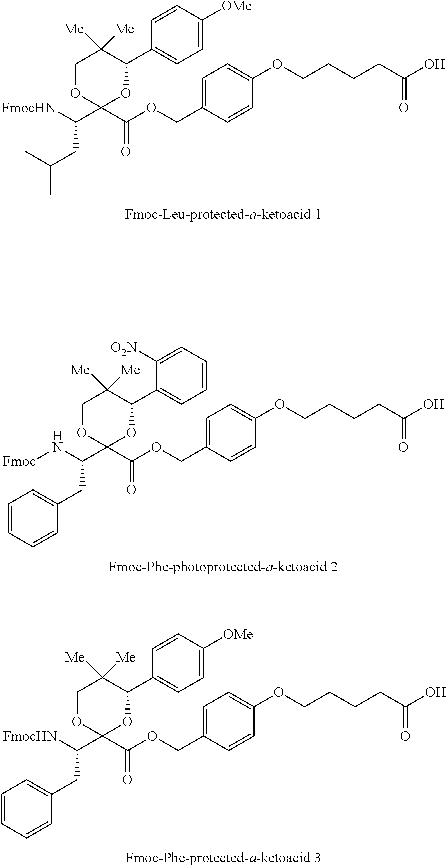

### Protected Ketoacid Used

Solid-phase peptide synthesis (SPPS): The peptide segments were synthesized on an automated peptide synthesizer using Fmoc-SPPS chemistry. The following Fmoc-amino acids with side-chain protecting groups were used: Fmoc-Ala-OH, Fmoc-Arg(Pbf)-OH, Fmoc-Asn(Trt)-OH, Fmoc-Asp(OtBu)-OH, Fmoc-Cys(Acm)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Nle-OH, Fmoc-Phe-OH, Fmoc-Pro-OH, Fmoc-Ser(tBu)-OH, Fmoc-Thr(tBu)-OH, Fmoc-Tyr (tBu), Fmoc-Trp(Boc)-OH, Fmoc-Val-OH and Fmoc or Boc-Opr-OH (Opr=5-(S)-oxaproline). Fmoc-pseudoproline dipeptides were incorporated in the synthesis if necessary. Fmoc deprotection reactions were performed with 20% piperidine in DMF or NMP containing 0.1 M Cl-HOBt (2×2 min). Coupling reactions were performed with Fmoc-amino acid (3.0-8.0 equiv to resin substitution), HCTU or HATU (2.9-8 equiv) as coupling reagents and DIPEA or NMM (6-16 equiv) in DMF or NMP at room temperature. The solution containing the reagents was added to the resin and allowed to react for 15 min, 30 min, or 2 h depending on the amino acid. Double coupling reactions were performed as needed. Unreacted free amines were capped using 20% acetic anhydride in DMF or NMP and 0.8 M NMM in DMF or NMP.

Resin cleavage and side chain deprotection. Once the peptide synthesis was completed, the peptides were cleaved from the resin using a cleavage cocktail at room temperature for 2 h. The resin was filtered off, and the filtrate was concentrated and treated with cold diethyl ether, triturated and centrifuged. The ether layer was carefully decanted, the residue was suspended again in diethyl ether, triturated and centrifuged. Ether washings were repeated twice. The resulting crude peptide was dried under vacuum and stored at −20° C. An aliquot of the solid obtained was solubilized in 1:1 CH₃CN/H₂O with 0.1% TFA (v/v) and analyzed by analytical RP-HPLC using C18 column (3.6 μm, 150×4.6 mm) at 60° C. The molecular weight of the product was identified using MALDI-TOF or LC-MS.

Purification of the peptides: Peptide segments, ligated peptides and linear proteins were purified by RP-HPLC. Different gradients were applied for the different peptides. The mobile phase was MilliQ-H₂O with 0.1% TFA (v/v) (Buffer A) and HPLC grade CH₃CN with 0.1% TFA (v/v) (Buffer B). Preparative HPLC was performed on a (50×250 mm) or on a C18 column (50×250 mm) at a flow rate of 40 mL/min at 40° C. or 60° C.

Purification: The peptide fragments purification was performed on standard preparative HPLC instruments. Preparative HPLC was performed on C18 column (5 m, 110A, 50×250 mm) at a flow rate of 40 mL/min on C18 column (5 m, 110A, 20×250 mm) or C4 column (5 m, 300A, 20.0×250 mm) at a flow rate of 10 mL/min. For both columns, room temperature, 40° C., or 60° C. were used during the purification. The mobile phase was MilliQ-H₂O with 0.1% TFA (v/v) (Buffer A) and HPLC grade CH₃CN with 0.1% TFA (v/v) (Buffer B).

Characterization of the peptides: Peptides and proteins were characterized by high resolution Fourier-transform mass spectrometry (FTMS) using a SolariX (9.4T magnet) spectrometer (Bruker, Billerica, USA) equipped with a dual ESI/MALDI-FTICR source, using 4-hydroxy-α-cyanocinnamic acid (HCCA) as matrix. Peptides segments, ligated peptides and linear proteins were analyzed by RP-HPLC using analytical HPLC instruments using standard C₄ column (3.6 μm, 150×4.6 mm) at room temperature or standard C18 column (3.6 μm, 150×4.6 mm) with a flow rate of 1 mL/min at 50° C. The peptide fragments were analyzed using a gradient of 20% B to 95% B in 12 min (Method A), 10% B to 85% B in 12 min (Method B) or 10% B to 95% B in 12 min (Method C).

### Example 2A—Preparation of a Synthetic IL-7 Polypeptide of SEQ ID NO: 3

### Segment 1: IL-7(1-34)-Leu-α-ketoacid (SEQ ID NO: 5)

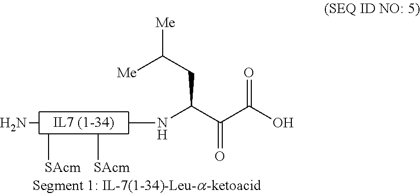

SEQ ID NO: 5 was synthesized on a 0.2 mmol scale on Rink Amide MBHA resin pre-loaded with Fmoc-Leu-protected-α-ketoacid (description in the general methods) (0.8 g) with a substitution capacity of ˜0.25 mmol/g.

Automated Fmoc-SPPS of SEQ ID NO: 5: The peptide elongation cycles including amino acid coupling, capping and Fmoc deprotection were performed as described in the general methods.

After the peptide elongation, the resin was washed with DCM and dried under vacuum. The mass of the dried peptidyl resin was 1.6 g. The peptide was cleaved from the resin using a mixture of 95:2.5:2.5 TFA:DODT:H₂O (10 mL/g resin) at room temperature for 2.0 h. The compound was precipitated as described in the general methods. 702 mg of crude peptide were obtained.

Purification of crude SEQ ID NO: 5 was performed by preparative HPLC using a C18 column (5 m, 110 Å, 250×50 mm) at a flow rate of 40 mL/min at 60° C. with a gradient of 30 to 80% B in 25 min. The fractions containing the purified product were pooled and lyophilized to obtain SEQ ID NO: 5 as a white solid in 94% purity. The isolated yield based on the resin loading was 17% (135 mg). MS (ESI): C₁₇₁H₂₈₁N₄₃O₆₃S₂; Average isotope calculated 1337.9940 Da [M+H]³⁺; found: 1337.9933 Da [M+H]³⁺. Retention time (analytical Method A): 5.66 min.

### Segment 2: Opr-IL-7(37-74)-Phe-photoprotected-α-ketoacid (SEQ ID NO: 7)

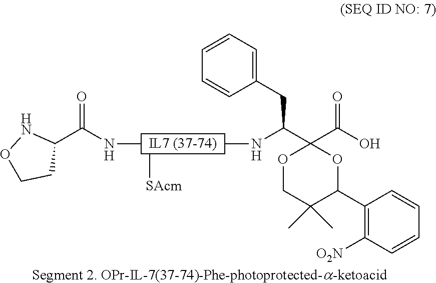

SEQ ID NO: 7 was synthesized on a 0.2 mmol scale on Rink Amide MBHA resin pre-loaded with Fmoc-Phe-photoprotected-α-ketoacid (description in the general methods) with a substitution capacity of 0.25 mmol/g.

The peptide elongation cycles including amino acid coupling, capping and Fmoc deprotection were performed as described in the general methods.

After the peptide elongation, the resin was washed with DCM and diethyl ether and dried under vacuum. The mass of the dried peptidyl resin was 2.2 g. The peptide was cleaved from the resin using a mixture of 95:2.5:2.5 TFA/DODT/H₂O (15 mL/g resin) at room temperature for 2.0 h. The compound was precipitated as described in the general methods. 1.2 g of crude peptide were obtained.

Purification of crude SEQ ID NO: 7 was performed by preparative HPLC using a C18 column (5 m, 110 Å, 250×50 mm) at a flow rate of 40 mL/min at 40° C. using CH₃CN/H₂O with a gradient of 10 to 60% B in 30 min. The fractions containing the purified product were pooled and lyophilized to obtain SEQ ID NO: 7 as a white solid in 97% purity. The isolated yield based on the resin loading was 20% (203 mg). RMS (ESI): C₂₃₄H₃₅₂N₆₅O₆₂S; m/z calculated: 5098.6114 Da [M+H]⁺; found: 5098.6026 Da [M+H]⁺. Retention time (analytical Method A): 6.30 min.

### Segment 3: Fmoc-Opr-IL-7(77-112)-Leu-α-ketoacid (SEQ ID NO: 9)

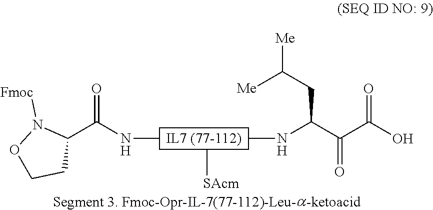

SEQ ID NO: 9 was synthesized on a 0.1 mmol scale on Rink Amide resin pre-loaded with Fmoc-Leu-protected-α-ketoacid (description in the general methods) with a substitution capacity of ˜0.29 mmol/g. 345 mg of resin was swollen in DMF for 15 min.

Automated Fmoc-SPPS of SEQ ID NO: 9: The peptide elongation cycles including amino acid coupling, capping and Fmoc deprotection were performed as described in the general methods.

After the peptide elongation, the resin was washed with DCM and dried under vacuum. The mass of the dried peptidyl resin was 0.94 g. The peptide was cleaved from the resin using a mixture of 95:2.5:2.5 TFA:DODT:H₂O (10 mL/g resin) at room temperature for 2.0 h. The compound was precipitated as described in the general methods. 473 mg of crude peptide were obtained.

Purification of crude SEQ ID NO: 9 was performed by preparative HPLC using a C18 column (5 μm, 110 Å, 50×250 mm) at a flow rate of 40 mL/min at 40° C. with a gradient of 10 to 50% B in 40 min. The fractions containing the purified product were pooled and lyophilized to obtain SEQ ID NO: 9 as a white solid in 98% purity. The isolated yield based on the resin loading was 25% (107 mg). HRMS (ESI): C₁₉₃H₃₁₅N₅₁O₅₇S; Average isotope calculated 4294.0030 Da [M+H]; found: 4293.2962 Da. Retention time (analytical Method B): 6.29 min.

### Segment 4: Opr-IL-7(115-152) (SEQ ID NO: 10)

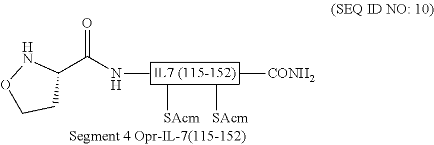

SEQ ID NO: 10 was synthesized on a 0.1 mmol scale on Rink Amide MBHA resin with a substitution capacity of ˜0.34 mmol/g. 294 mg of resin was swollen in DMF for 15 min. Automated Fmoc-SPPS of SEQ ID NO: 10: The peptide elongation cycles including amino acid coupling, capping and Fmoc deprotection were performed as described in the general methods.

The resin was washed with DCM and dried under vacuum. The mass of the dried peptidyl resin was 725 mg. The peptide was cleaved from the resin using a mixture of 92.5:2.5:2.5:2.5 TFA:TIPS:DODT:H₂O (10 mL/g resin) at room temperature for 2.0 h. The compound was precipitated as described in the general methods. 145 mg of crude peptide were obtained.

Purification of crude SEQ ID NO: 10 was performed by preparative HPLC using a Gemini NX-C18 110 Å column (5 m, 50×250 mm) at a flow rate of 40 mL/min at 40° C. with a gradient of 10 to 50% B in 40 min. The fractions containing the purified product were pooled and lyophilized to obtain SEQ ID NO: 10 as a white solid in 98% purity. The isolated yield based on the resin loading was 8% (40 mg). IRMS (ESI): C₂₁₅H₃₆₁N₆₁O₆₀S₂; Average isotope calculated 4823.6568 Da [M+H]; found: 4823.6542 Da. Retention time (analytical Method B): 6.15 min.

### Segment 12: IL-7-Seg12 Preparation (SEQ ID NO: 12)

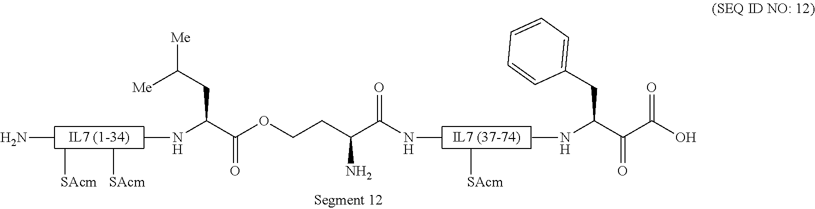

SEQ ID NO: 5 (17.5 mg; 4.36 μmol; 1.1 equiv) ketoacid and SEQ ID NO: 7 (20 mg; 3.92 μmol; 1.0 equiv) were dissolved in 15 mM DMSO:H₂O (9.5:0.5) containing 0.1 M oxalic acid (241 μL). A very homogeneous liquid solution was obtained. The ligation vial was protected from light and the mixture was heated overnight at 60° C. After completion of the ligation, the mixture was diluted with 1:1 CH₃CN:H₂O with 0.1% TFA (v/v) (4 mL), and the mixture was irradiated at a wavelength of 365 nm for 1.5 h to allow photodeprotection of the C-terminal ketoacid. The reaction mixture was further diluted with 1:1 CH₃CN/H₂O (q.s. 10 mL) with TFA (0.1%, v v).

The diluted mixture was filtered and injected into preparative HPLC. Crude ligated peptide was purified by preparative HPLC using a C18 column (5 m, 50×250 mm) at a flow rate of 40 mL/min at 60° C., with a 2-step gradient: 10 to 40% B in 5 min, then 40 to 70% Bin 30 min. The fractions containing the purified product were pooled and lyophilized to obtain SEQ ID NO: 12 as a white solid in 98% purity. The isolated yield was 38% (13.1 mg). MS (ESI): C₃₉₃H₆₁₉N₁₀₇O₁₂₀S₃; m/z calculated: 8858.4917 Da [M+H]⁺; found: 8858.4928 Da [M+H]⁺. Retention time (analytical Method B): 5.41 min.

### Segment 34: IL-7-Seg34 Preparation (SEQ ID NO: 13)

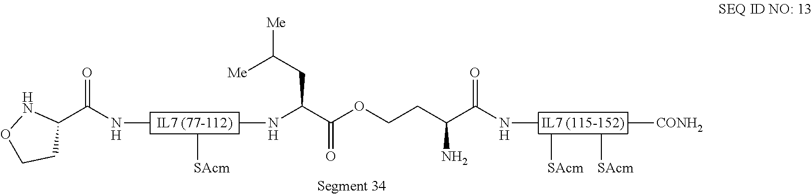

Peptide ketoacid SEQ ID NO: 9 (55.0 mg; 12.8 μmol; 1.2 equiv) and hydroxylamine peptide SEQ ID NO: 10 (51.5 mg; 10.6 μmol; 1.0 equiv) were dissolved in 9:1 DMSO/H₂O containing 0.1 M oxalic acid (530 μL). A very homogeneous liquid solution was obtained. It was let to react The reaction was heated overnight at 60° C. Upon completion of the ligation reaction, the mixture was diluted with DMSO (1060 μL). Fmoc deprotection was performed initiated by adding diethylamine (80 μL, 5%, v v) at room temperature for 15 min. A second portion of diethylamine (80 μL) in DMSO (1590 μL) was added to the reaction mixture, and the resulting mixture was reacted that was stirred at room temperature for another 15 min.

Trifluoroacetic acid (160 μL) was added in order to neutralize the reaction mixture. A very homogeneous and colorless liquid solution was obtained. The resulting mixture was further diluted with 1:1 CH₃CN/H₂O (q.s. 15 mL) with TFA (0.1%, v v). The diluted mixture was filtered and purified by preparative HPLC using a C18 column (5 m, 250×50 mm) at a flow rate of 40 mL/min at 40° C. with a gradient of 10% to 50% B in 40 min. The fractions containing the purified product were pooled and lyophilized to obtain SEQ ID NO: 13 as a white solid in 92% purity. The isolated yield was 51.5 mg (55%). HRMS (ESI): C₃₉₂H₆₆₆N₁₁₂O₁₁₃S₃; Average isotope calculated 8851.9104 Da [M+H]; found: 8851.8897 Da. Retention time (analytical Method B): 6.08 min.

### Preparation of SEQ ID NO: 3-Seg1234 with Acm (SEQ ID NO: 3)

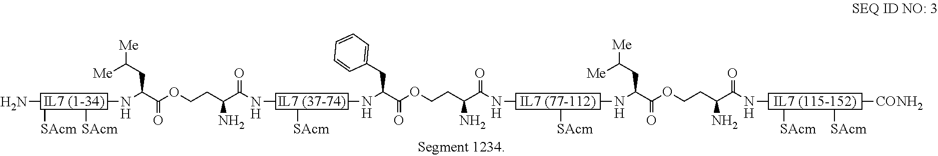

Peptide ketoacid SEQ ID NO: 12 (17.4 mg; 1.96 μmol; 1.2 equiv) and hydroxylamine peptide SEQ ID NO: 13 (14.5 mg; 1.64 μmol; 1.0 equiv) were dissolved in DMSO:H₂O (9.5:0.5) containing 0.1 M oxalic acid (110 μL, 15 mM peptide concentration). A homogeneous liquid solution was obtained, and the solution was heated overnight at 60° C.

After completion of the ligation the mixture was diluted with 1:1 H₂O/CH₃CN (q.s. 8 mL) containing TFA (0.1%, v v). The diluted mixture was filtered and injected into preparative HPLC. Crude ligated peptide was purified by preparative HPLC using a C18 column (5 m, 250×50 mm) at a flow rate of 40 mL/min at 60° C. using with a gradient of 30 to 80% B in 30 min. The fractions containing the purified product were pooled and lyophilized to obtain SEQ ID NO: 3 (tri-depsipeptide) with Acm as a white solid in 99% purity. The isolated yield was 28% (8 mg). HRMS (ESI): C₇₈₄H₁₂₈₅N₂₁₉O₂₃₁S₆; Average isotope calculated 17666.4121 Da [M]; found: 17666.4233 Da. Retention time (analytical Method C): 5.33 min.

### Acm Deprotection for the Preparation of SEQ ID NO: 3-Linear Protein: IL-7-Linear Protein (SEQ ID NO: 3)

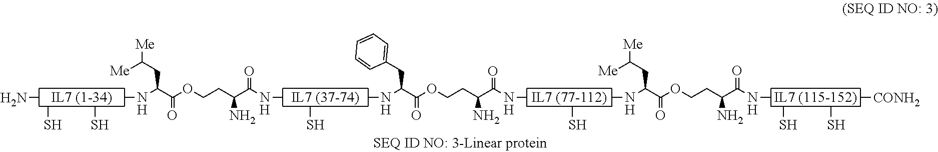

SEQ ID NO: 3 (5.8 mg; 0.33 μmol) was dissolved in AcOH:H₂O (1:1) (1.3 mL, 0.25 mM protein concentration) and silver acetate (13 mg, 1%, m v) was added to the solution. The mixture was shaken for 2.5 h at 50° C. protected from light.

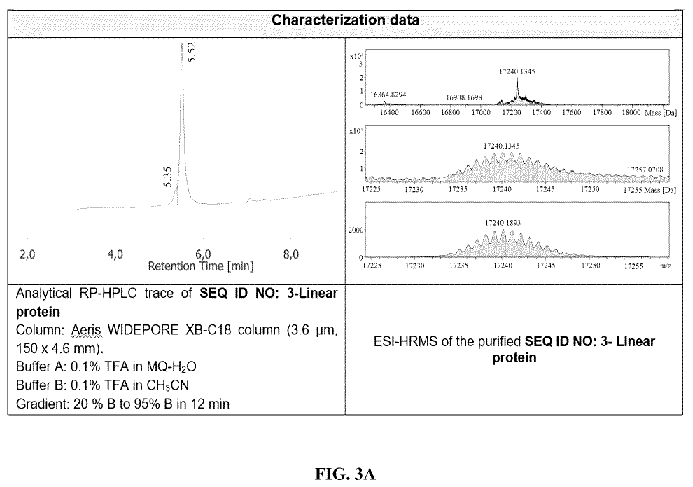

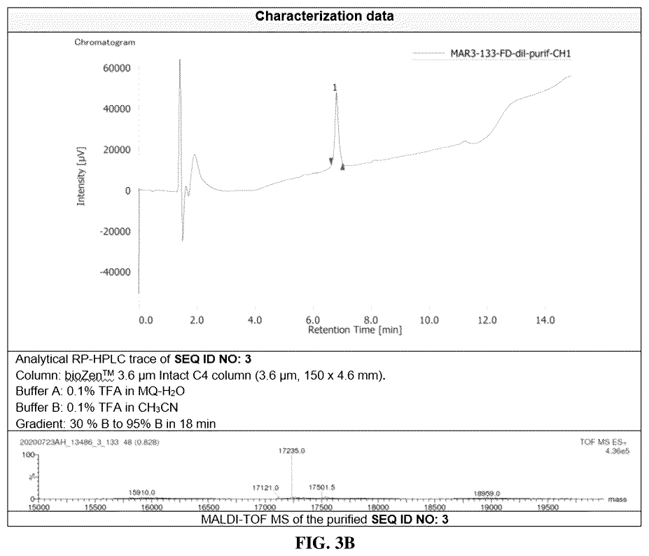

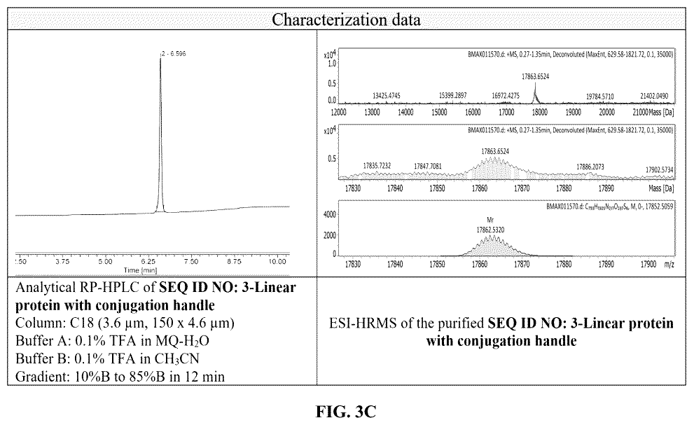

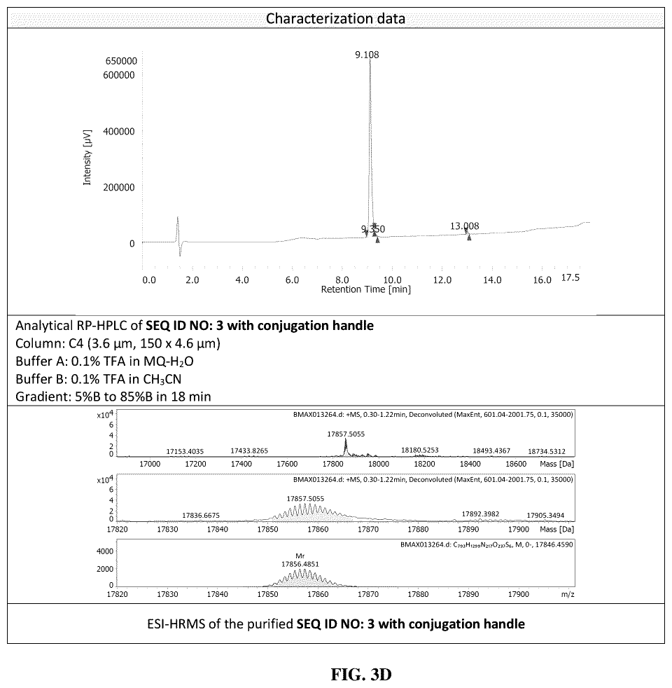

After completion of reaction, the sample was diluted with 1:1 CH₃CN:H₂O with 0.1% TFA (v/v). The sample was purified by preparative HPLC on a C18 column (5 μm, 110A, 250×20 mm) at a flow rate of 10 mL/min at room temperature using CH₃CN/H₂O with 0.1% TFA (v/v) as mobile phase, with a two-step gradient: 10 to 30% CH₃CN in 5 min and 30 to 95% CH₃CN in 20 min. The fractions containing the purified product were pooled and lyophilized to obtain 2.8 mg SEQ ID NO: 3-Linear protein as a white powder in 98% purity. (49% yield for Acm deprotection and purification steps). MS (ESI): C₇₆₆H₁₂₅₅N₂₁₃O₂₂₅S₆; m/z calculated: 17240.1893 Da [M+H]; found: 17240.1636 Da [M+H]. FIG. 3A shows characterization data (HPLC, ESI-HRMS) for SEQ ID NO: 3-Linear protein. Retention time (analytical Method A): 5.52 min.

SEQ ID NO: 3-Folded Protein: Rearrangement and Folding of IL-7 Linear Protein.

2.3 mg (0.133 μmol) of the linear IL-7 protein were dissolved in 7.5 mL of 50 mM Tris buffer containing 6 M GnHCl, 50 mM NaCl, 1 mM EDTA and 2 mM CysHCl (18 μM protein concentration), which was adjusted to pH 8.0 by adding a solution of 6 M aqueous HCl. The mixture was gently shaken at rt for 2 h. The rearrangement was monitored by analytical reverse phase HPLC.

The solution with the rearranged protein was cooled to 4° C. and diluted (×3) with 15 mL of 50 mM Tris buffer containing 50 mM NaCl and 0.1 M Arg, which was adjusted to pH 8.0 by adding a solution of 6 M aqueous HCl. The folding was allowed to proceed for 48 h at 4° C. The folding was monitored according to the rearrangement monitoring conditions.

After completion of folding reaction as ascertained by HPLC, the sample was acidified with TFA to pH=3, and purified by preparative HPLC using a C4 column (5 μm, 20×250 mm) at a flow rate of 10 mL/min at rt with a gradient of 30 to 85% B in 50 min. The fractions containing the purified product were pooled and lyophilized to obtain 0.8 mg of the folded IL-7 polypeptide (35% yield) as a white powder. The purity and identity of the pure folded protein was further confirmed by analytical HPLC and ESI/MS. MS (ESI): C₇₆₆H₁₂₄₉N₂₁₃O₂₂₅S₆; m/z calculated: 17235.0 Da [M+H]⁺; found: 17235.0 Da [M+H]⁺. FIG. 3B shows characterization data of folded SEQ ID NO: 3 IL-7 protein.

### Example 2B—N-Terminal Modified Synthetic IL-7 (SEQ ID NO: 3 with Azide Conjugation Handle in N-Terminus (Composition AA))

The method for synthesizing IL-7 according to Example 2A is modified in order to prepare construct SEQ ID No: 3 with azide conjugation handle in N-terminus (Composition AA). Composition AA differs from the IL-7 polypeptide of SEQ ID NO: 3 prepared in Example 2A (i.e., SEQ ID NO: 3) in that Composition AA contains a modified N-terminal amine having a structure

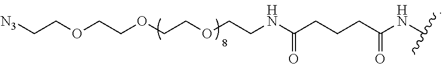

This version is prepared analogously to the IL-7 of SEQ ID NO: 3 in example 2A above with the following modification performed after final Fmoc deprotection of the N-terminal residue. Manual coupling reaction is performed at r.t. for 2 h by addition of glutaric anhydride (CAS RN 108-55-4, 114.10 mg, 5 equiv.) and DIPEA (242 μL, 7 equiv.) in DMF to the resin. Secondly, coupling with commercially available O-(2-Aminoethyl)-O′-(2-azidoethyl) nonaethylene glycol (Compound 2, 421 mg, 4 equiv) in DMF is performed at r.t. for 3 hours by addition of DIPEA (276 μL, 8 equiv) and HATU (300.4 mg, 3.95 equiv) in DMF to the resin.

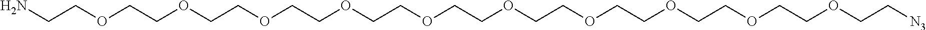 [SMILES: [N-]=[N+]=NCCOCCOCCOCCOCCOCCOCCOCCOCCOCCOCCN]

### O-(2-Aminoethyl)-O′-(2-azidoethyl) nonaethylene glycol (Compound 2)

The resin is then washed and cleaved as per the normal protocol, and the modified N-terminal fragment is used in the remaining ligation steps as described in Example 2A.

### Example 3—Additional Protocol for Folding of IL-7

Folding Step 1: The linear protein (e.g., tri-depsipeptide version of the final sequence) is dissolved in 50 mM Tris buffer, containing 6 M GnHCl, 50 mM NaCl, 1 mM EDTA and 10 mM CysHCl (40 μM protein concentration), which is adjusted to pH 8.0 by adding a solution of 6 M aqueous HCl. The mixture is gently shaken at rt for 3 h. The rearrangement is monitored by analytical reverse phase HPLC.

Folding Step 2: The solution with the rearranged protein is cooled to 4° C. and diluted (×8) with 50 mM Tris buffer containing 50 mM NaCl, 0.11 M Arg, 1 mM EDTA and 0.142 mM cystine, which is adjusted to pH 8.0 by adding a solution of 6 M aqueous HCl. The folding is performed for 20 h at 4° C. and monitored by HPLC.

Purification: After completion of the folding reaction as ascertained by HPLC, the sample is acidified with TFA to pH=3, and purified by preparative HPLC using a C4 column (20×250 mm) at a flow rate of 10 mL/min at rt using CH₃CN/H₂O with 0.1% TFA (v/v) as mobile phase, with a gradient of 30 to 85% CH₃CN with 0.1% TFA (v/v) in 50 min. The fractions containing the purified product are pooled and lyophilized with 5% (w/v) sucrose to obtain the folded IL-7 syntein. The purity and identity of the pure folded protein is further confirmed by analytical HPLC and LC/ESI/MS/MS.

### Example 4: Determination of IL-7-Induced pSTAT5 Phosphorylation of Synthetic IL-7

Primary pan T-cells were obtained from healthy donor buffy coats by peripheral blood mononuclear cell (PBMC) purification using Ficoll gradient centrifugation, followed by negative isolation with magnetic beads and then cryopreserved until use. Pan T-cells were thawed, allowed to recover overnight in T-cell medium (RPMI 10% FCS, 1% Glutamine, 1% NEAA, 25 μM bMeoH, 1% NaPyrovate). After two washing steps with PBS, cells were resuspended in PBS. Cells were then distributed at 200′000 cells per well and stimulated with serial dilutions of wild type or modified IL-7 polypeptides for 40 min at 37° C./5% CO₂. After incubation, cells were fixed and permeabilized using the Transcription Factor Phospho Buffer kit followed by staining of surface and intracellular staining markers (CD4, CD8, CD25, FoxP3, CD45RA, pStat5) to enable the identification of cell subsets and to measure levels of STAT5 phosphorylation. FACS measurement was done either with a NovoCyte or a Quanteon Flow Cytometer from Acea Biosciences. Flow-Jo was used for all FACS analyses.

The percentage of pSTAT5-positive T-cell subsets was plotted against concentrations of either wild type or modified IL-7 polypeptides. The half maximal effective concentration (EC50) was calculated based on a variable slope, four parameter analysis. TABLE 4 shows the gating strategy for T-cell subset identification.

TABLE 4

| | |
|---|---|
| CD4 Treg | CD4+, CD45RA−, CD25^{Hi}, FoxP3+ |
| CD4 Memory | CD4+, CD45RA− |
| CD4 Naïve | CD4+, CD45RA+ |
| CD8 Memory | CD8+, CD45RA− |
| CD8 Naïve | CD8+, CD45RA+ |

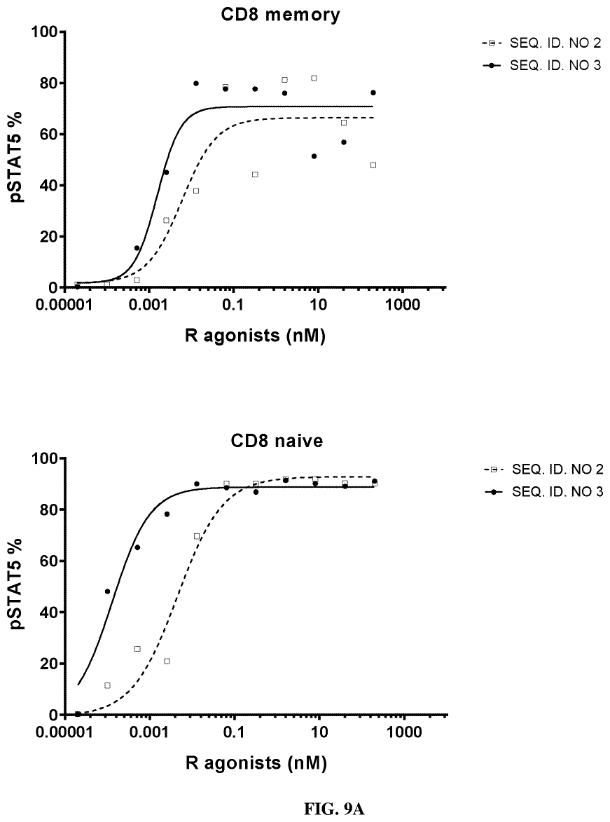

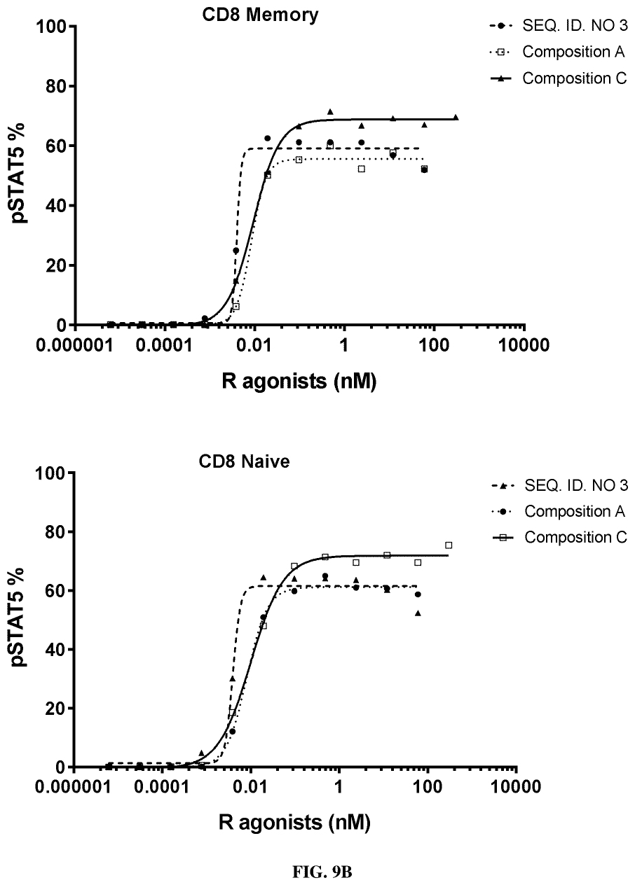

pSTAT5 induction in primary human T cells for WT IL-7 His (SEQ ID NO: 2) and synthetic IL-7 (SEQ ID NO: 3) is shown in FIG. 9A show dose dependent pSTAT5 induction in CD8 naïve, and CD8 memory T cells when treated with WT IL-7 His (SEQ ID NO: 2) or synthetic IL-7 of SEQ ID NO: 3. WT IL-7 His and synthetic IL-7 exhibit similar ability to induce pSTAT5, indicating that synthetic IL-7 behaves similar to wild type. FIG. 9B shows dose dependent pSTAT5 induction in CD8 naïve and CD8 memory T cells treated with SEQ. ID. NO 3, Composition A and Composition C. Both Composition A and Composition C show similar activity to SEQ. ID. NO 3, indicating that conjugation does not impact the IL-7 functionality. EC50 values for various T cell subtypes are shown in Table 5 below.

TABLE 5

| | | SEQ. ID. | SEQ. ID. | Composition | Composition | Composition |
|---|---|---|---|---|---|---|
| | | NO 2 | NO 3 | A | B | C |
| Avg | CD4 | 0.001 | 0.005 | 0.004 | 0.005 | 0.018 |
| EC50 | Memory | | | | | |
| (nM) | CD4 Naive | 0.001 | 0.005 | 0.004 | 0.003 | 0.016 |
| (human | CD8 | 0.006 | 0.006 | 0.006 | 0.007 | 0.032 |
| pSTAT5) | Memory | | | | | |
| | CD8 Naive | 0.002 | 0.006 | 0.004 | 0.007 | 0.015 |
| | CD4 Treg | 0.033 | 0.015 | 0.011 | 0.017 | 0.034 |

### Example 5: Preparation of Anti-PD-1 Antibody- (IL-7) Immunocytokine

An anti-PD-1 antibody (e.g., Pembrolizumab or LZM-009) and Composition AA are used to prepare an immunocytokine with the following exemplary methods. FIG. 5A illustrates site selective introduction of a conjugation handle on a Fc domain.

A conjugatable variant of Pembrolizumab or LZM-009 is prepared using an AJICAP™ method (Ajinomoto Bio-Pharma Services). This method allows production of >50 mg of conjugatable Pembrolizumab antibody within weeks. The conjugatable product harbors one or two chemical handles for further modifications (FIG. 5B). Alternatively, a mixture of DAR1 and DAR2 antibody immunoconjugates is generated, and the average cytokine loading is used to determine an intermediate DAR (e.g., DAR 1.1, 1.2, 1.3, 1.4, 1.5, 1.6, 1.7, 1.8, or 1.9). General protocols for the AJICAP™ methodology are found at least in PCT Publication No. WO2018199337A1, PCT Publication No. WO2019240288A1, PCT Publication No. WO2019240287A1, PCT Publication No. WO2020090979A1, Matsuda et al., Mol. Pharmaceutics 2021, 18, 4058-4066, and Yamada et al., AJICAP: Affinity Peptide Mediated Regiodivergent Functionalization of Native Antibodies. Angew. Chem., Int. Ed. 2019, 58, 5592-5597, and in particular Examples 2-4 of US Patent Publication No. US20200190165A1. A general protocol for this methodology is provided below:

A modified antibody (e.g., an anti-PD-1 antibody such as Pembrolizumab or LZM-009) comprising a DBCO conjugation handle is prepared using a protocol modified from Examples 2-4 of US Patent Publication No. US20200190165A1. Briefly, the anti-PD-1 antibody with a free sulfhydryl group attached to a lysine residue side chain in the Fc region is prepared by contacting the antibody with an affinity peptide configured to deliver a protected version of the sulfhydryl group (e.g., a thioester or disulfide) to the lysine residue. An exemplary peptide capable of performing this reaction is shown below, as reported in Matsuda et al., Mol. Pharmaceutics 2021, 18, 4058-4066, which selectively attached the sulfhydryl group via the NHS ester at residue K248 of the Fc region of the antibody:

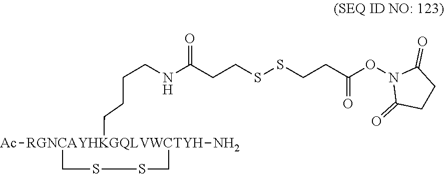

Alternative affinity peptides targeting alternative residues of the Fc region are described in the references cited above for AJICAP™ technology, and such affinity peptides can be used to attach the desired functionality to an alternative residue of the Fc region (e.g., K246, K288, etc.). For example, the disulfide group of the above affinity peptide could instead be replaced with a thioester to provide an sulfhydryl protecting group (e.g., the relevant portion of the affinity peptide would have a structure of

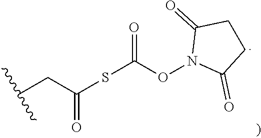 [SMILES: *CC(=O)SC(=O)ON1C(=O)CCC1=O]

The protecting group (e.g., the disulfide or thioester) is then removed to reveal the free sulfhydryl (e.g., by reduction of a disulfide with TCEP or hydrolysis). The free sulfhydryl is then reacted with a bifunctional reagent comprising a bromoacetamide group connected to the DBCO conjugation handle through a linking group (e.g., bromoacetamido-dPEG®₄-amido-DBCO). The method can be used to produce an antibody with one DBCO group present (DAR1) and/or two DBCO groups attached to the antibody (DAR2, one DBCO group linked to each Fc of the antibody). The desired azide modified IL-7 polypeptide (e.g., Composition AA) is then reacted with the DBCO modified antibody to produce the immunocytokine.

In another embodiment, antibody comprising a single DBCO conjugation handle is prepared by first reacting excess anti-PD-1 antibody with appropriately loaded affinity peptide to introduce a single sulfhydryl after appropriate removal of protecting group (e.g., disulfide reduction or thioester cleavage). A bifunctional linking group with a sulfhydryl reactive conjugation handle and DBCO conjugation handle (e.g., bromoacetamido-dPEG®₄-amido-DBCO) is then reacted with the single sulfhydryl to produce the single DBCO containing antibody. The single DBCO containing antibody is then conjugated with a suitable azide containing IL-7 (e.g., Composition AA) to achieve an anti-PD-1-IL-7 immunoconjugate with a DAR of 1.

### Conjugation of Antibody to IL-7 Polypeptide

The DBCO modified antibody is then conjugated to an IL-7 polypeptide comprising an azide moiety at a desired point of attachment (e.g., Composition AA). DBCO modified antibody with one (DAR1) or two (DAR2) reactive handles are reacted with 2-10 equivalents of azide containing IL-7 (pH 5.2 buffer, 5% trehalose, rt, 24 h). In an alternative embodiment, antibody comprising two DBCO conjugation handles is reacted either as an excess reagent (e.g., 5-10 equivalents) with 1 equivalent of SEQ. ID. NO 3 comprising an azide functionality to produce a DAR1 antibody or the antibody comprising two DBCO conjugation handles is reacted with 1 equivalent of antibody with excess reagent of SEQ. ID. NO 3 comprising an azide (e.g., 5-10 equivalents) to produce a DAR2 antibody.

Conjugatable variants of anti-PD-1 antibody with one (DAR1) or two (DAR2) reactive handles are reacted with 1 equivalent, 2-10 equivalents, or 5-10 equivalents of a capped mAB (pH 5.2 buffer, 5% trehalose, rt, 24 h). The resulting conjugate is purified by cation-exchange chromatography and/or size exclusion chromatography approximately 50-60% yield.

The anti-PD-1 antibody-IL7 conjugate is purified from unreacted starting product and aggregates using a desalting column, CIEX and SEC (GE Healthcare Life Sciences AKTA pure, mobile phase: Histidine 5.2/150 mM NaCl/5% Trehalose, column: GE Healthcare Life Sciences SUPERDEX™ 200 increase 3.2/300, flow rate: 0.5 mL/min).

The purity and identity of the conjugate is confirmed by RP-HPLC (HPLC: ThermoFisher Scientific UHPLC Ultimate 3000, column: Waters BEH C-4 300A, 3.0 μm, 4.6 mm, 250 mm, mobile phase A: 0.05% TFA in Water, mobile phase B: 0.05% TFA in mixture of ACN:IPA:ETOH:H2O (5:1.5:2:1.5), flow rate: 0.5 mL/min, injection amount: 10 μg (10 pL Injection of 1 mg/mL), gradient: 0% to 20% mobile phase B in 50 min) and SDS-PAGE.

### Example 6: ELISA Assays with PD-1 Antibodies and Conjugates to IL-7

IL-7 syntein Composition AA was conjugated to anti-PD-1 antibody Pembrolizumab (DAR1 (Composition A) and DAR2 (Composition B)) and LZM009 (DAR1) (Composition C). Each of these variant conjugates and unmodified antibodies were assayed by ELISA for their ability to bind to human PD-1 according to the following protocol.

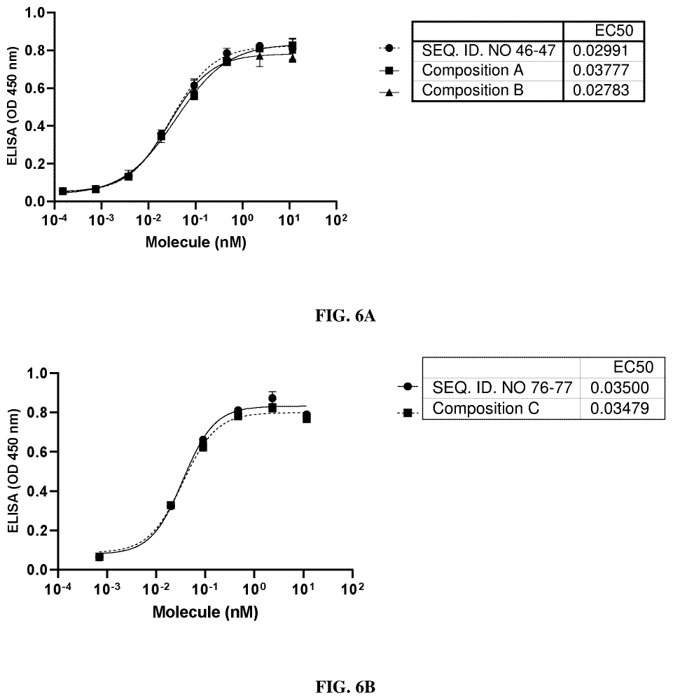

Materials: ELISA plate were Costar Assay plate 96 well clear Flat bottom Half Area High binding Polystyrene, CORNING #3690. Biotinylated Recombinant Human PD-1 was (CD279)-Fc Chimera (carrier-free), Biolegend #789406. Streptavidin-HRP was Sigma #RABHRP3. TMB solution was 3,3′,5,5′-Tetramethylbenzidine (Sigma T0440). Stop solution was Sigma #CL07STOP solution (0.5M H2SO4). Buffers were: Coating buffer was PBS. Wash buffer was PBS-0.02% Tween20. Blocking buffer was PBS-0.02% Tween20 1% BSA. Protein diluent was PBS-0.02% Tween20 0.1% BSA. STOP solution was 0.5M H2SO4. Procedure: Immunocytokines and parental antibody were coated overnight at 4° C. The ELISA plates were washed 4 times with 100 μl PBS—0.02% Tween20. and blocked with PBS—1% BSA. A serial dilution of h-PD1 Fc was prepared and plates were incubated for two hours at 37° C., with shaking (600 rpm). ELISA plates were washed and incubated with Streptavidin-HRP 30 min at RT, with shaking (600 rpm). ELISA plates were washed and incubated with ready-to-use TMB solution. Reaction was stopped and plates were read by OD450 on an Enspire plate reader. The results from this experiment are shown in FIG. 6, which shows that ability of the DAR1 and DAR2 Immunocytokine to bind PD-1 after conjugation of IL7 was not substantially altered compared to unmodified antibody. Similarly, conjugation of IL7 to anti-PD-1 antibody LZM-009 also did not substantially affect binding to PD-1 as shown in FIG. 6B.

### Example 7: PD-1/PD-L1 Blockade Assay

Using the PD1/PDL1 blocking assay from Invivogen (Cat No: rajkt-hpd1), he ability to still block the PD1/PDL1 interaction after conjugation of IL7 was assessed PD-1/PD-L1 blockade assay was done according to the protocol provided by Invivogen (Cat No: rajkt-hpd1) below.

Materials were Flat 96 well, Corning #3596 culture plates. Read out plates were flat white 96 well plates, ThermoSchientific #136102. Luciferase substrate assay solution was QUANTI-Luc (Invivogen #rep-qlc). Test cells were Jurkat-Lucia™ TCR-hPD-1 cells, (Invivogen #rajkt-hpd1). Target cells were Raji-APC-hPD-L1 cells (Invivogen #rajkt-hpd1).

For the assay procedure, the parental antibody and immunocytokine Composition C were diluted from a top concentration of 1p M in assay medium. A total of seven dilution steps were made by diluting the next higher antibody concentration 1:6. For each well 20 μl of molecules dilution was added, according to the assay layout. As a negative control, 20 ul of assay media containing no antibody or immunoconjugate was used.

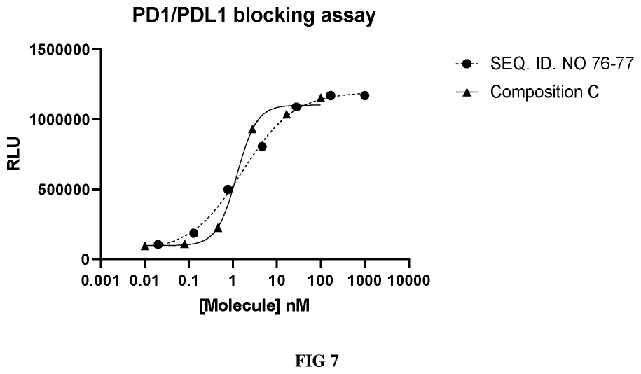

Jurkat-Lucia™ TCR-hPD-1 cells and Raji-APC-hPD-L1 cells were seeded and treated with a dose titration of the test molecules. After 24 hrs sample supernatants were analysed for Luciferase activity by adding 50 μl of QUANTI-Luc. The bioluminescence was measured immediately after the addition of the luciferase substrate using an Enspire plate reader. The results of this experiment comparing Composition C are shown in FIG. 7. Composition C and SEQ ID NOs: 76-77 exhibited similar ability to block the interaction of PD-1 and PD-L1.

### Example 8: FcRN Binding Assay

The ability of SEQ ID NOs: 76-77 and Composition C to bind human and mouse FcRN was determined using an AlphaLISA assay according to the below protocol.

Samples were assayed using AlphaLISA kit from PerkinElmer, cat #AL3095C . . . SEQ ID NOs: 76-77 and Composition C was serially diluted Human FcRn (4× concentrated) was diluted to a final concentration of 50 ng/ml in 1×MES buffer. Human IgG Conjugated Acceptor Beads (2×) and Streptavidin (SA) Donor Beads were diluted to reach final concentrations of 5 μg/ml. To wells in the assay plates, 10 ul of diluted antibodies, 10 ul of diluted FcRn, 20 ul of human IgG conjugated Acceptor beads, and SA-Donor beads were added. Plates were then incubated for 90 minutes at room temperature then measured using 680 nm excitation and 615 nm emission on a plate reader with the appropriate capabilities.

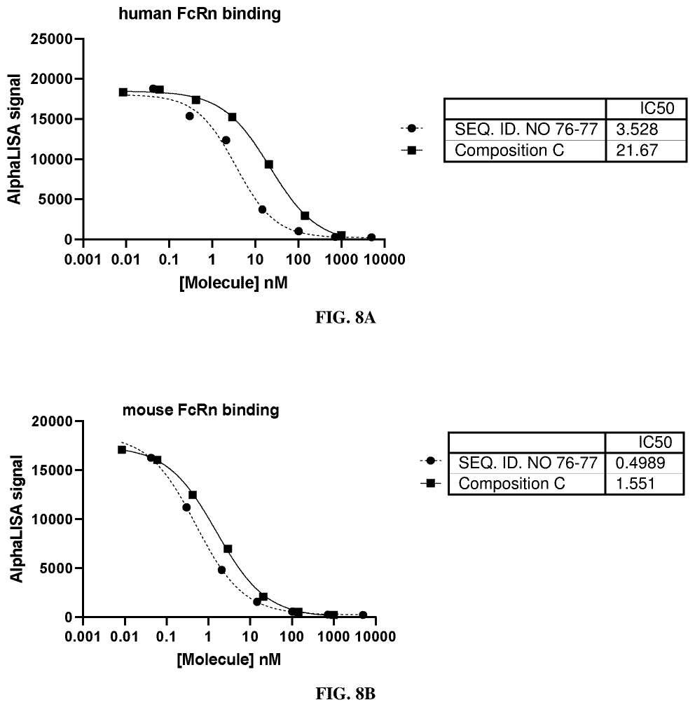

Results in FIG. 8A for human and in FIG. 8B for binding to mouse FcRn. Both SEQ ID NOs: 76-77 and Composition C showed similar binding curves for FcRn in the assay,

### Example 9:—In Vivo Tumor Growth Inhibition

An experiment was performed as described below in order to assess the anti-tumor properties of SEQ ID NOs: 76-77 and Composition C,

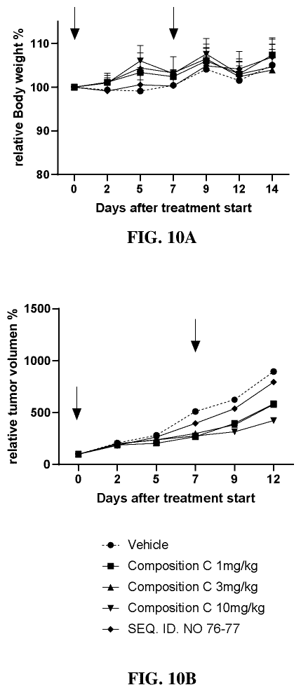

Transgenic BALBc-hPD1 mice (BALB/cJGpt-Pdcd1em1Cin(hPDCD1)/Gpt), which are genetically modified with knock-in of human PD-1 as sold by GemPharmatech (Cat #T002726) were implanted with the syngeneic CT26 tumor model and treated with SEQ ID NOs: 76-77 (10 mg/kg, once a week) and Composition C (1,3 and 10 mg/kg, once a week).
Body weight measurements and relative tumor volume for the various groups during the course of the study is shown in FIGS. 10A and 10B, respectively. As shown in FIG. 10A none of the dose levels of Composition C induced any body weight loss indicating a favorable safety profile of Composition C. As shown in FIG. 10B Composition C induces a dose dependent tumor growth inhibition that is superior to SEQ ID NOs: 76-77. In summary the in vivo data provided show that Composition C is safe, effective and superior to the parental antibody.

1. A composition comprising:
a polypeptide which selectively binds to programmed cell death protein 1 (PD-1);
an IL-7 polypeptide; and
a linker, wherein the linker comprises:
a first point of attachment covalently attached to the IL-7 polypeptide; and
a second point of attachment covalently attached to a non-terminal residue of the polypeptide which selectively binds to PD-1.

2. (canceled)

3. The composition of claim 1, wherein the first point of attachment is at an N-terminal residue of the IL-7 polypeptide.

4. The composition of claim 1, wherein the IL-7 polypeptide is synthetic.

5. The composition of claim 4, wherein the IL-7 polypeptide comprises homoserine residues at each of positions 36, 76, and 114, wherein residue position numbering is based on SEQ ID NO: 1 as a reference sequence.

6. The composition of claim 3, wherein IL-7 polypeptide comprises an amino acid sequence as set forth in SEQ ID NO: 3.

7. (canceled)

8. The composition of claim 1, wherein the polypeptide which selectively binds to PD-1 is an anti-PD-1 antibody or an antigen binding fragment.

9. (canceled)

10. The composition of claim 8, wherein the second point of attachment is at an amino acid residue in an Fc region of the polypeptide which selectively binds to PD-1.

11-13. (canceled)

14. The composition of claim 10, wherein the second point of position attachment is at the Fc region at a position of a K246 amino acid residue, a K248 amino acid residue, a K288 amino acid residue, a K317 amino acid residue, or a combination thereof (Eu numbering).

15. The composition of claim 14, wherein the second point of attachment is at the K248 amino acid residue.

16. The composition of claim 1, wherein the polypeptide which selectively binds to PD-1 is a monoclonal antibody.

17. (canceled)

18. The composition of claim 1, wherein the polypeptide which selectively binds to PD-1 comprises an IgG.

19. The composition of claim 18, wherein the IgG is an IgG1, an IgG4, or is derived therefrom.

20. (canceled)

21. The composition of claim 1, wherein the anti-PD1 polypeptide comprises Nivolumab, Pembrolizumab, LZM-009, Dostarlimab, Sintilimab, Spartalizumab, Tislelizumab, or Cemiplimab.

22-24. (canceled)

25. The composition of claim 1, wherein the linker comprises a polymer.

26-29. (canceled)

30. The composition of claim 1, wherein the linker comprises a structure 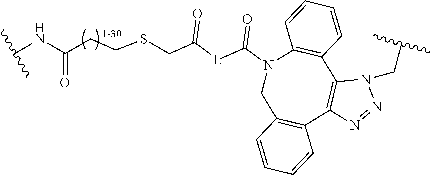
wherein 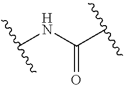 [SMILES: *NC(*)=O]
is the first point of attachment to a lysine residue of the polypeptide which selectively binds to PD-1;
L is a linking group; and 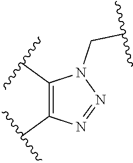 [SMILES: *Cn1nnc(*)c1*]
is a point of attachment to a linking group which connects to the first point of attachment.

31-65. (canceled)

66. A synthetic IL-7 polypeptide, comprising a homoserine (Hse) residue at a position selected from a region of residues 31-41, a region of residues 71-81, or a region of residues 109-119, wherein residue position numbering is based on SEQ ID NO: 1 as a reference sequence.

67. The synthetic IL-7 polypeptide of claim 66, wherein the synthetic IL-7 polypeptide comprises a Hse residue in each of the region of residues 31-41, the region of residues 71-81, and the region of residues 109-119.

68-70. (canceled)

71. The synthetic IL-7 polypeptide of claim 66, wherein the synthetic IL-7 polypeptide comprises Hse residues at each of positions 36, 76, and 114.

72-79. (canceled)

80. The synthetic IL-7 polypeptide of claim 66, wherein the synthetic IL-7 polypeptide is prepared from the ligation of four chemically synthesized fragments.

81-86. (canceled)

87. A method of making a synthetic TL-7 polypeptide, comprising:
a) synthesizing two or more fragments of the synthetic IL-7 polypeptide;
b) ligating the fragments; and
c) folding the ligated fragments.

88-105. (canceled)
